SEQUENCE LISTING (544 sequences)

SEQ ID NO: 1 - protein, 11 aa, Artificial Sequence
  Synthetic Construct
FTFTSYGIHWV

SEQ ID NO: 2 - protein, 13 aa, Artificial Sequence
  Synthetic Construct
FSLSTSGVAVSWI

SEQ ID NO: 3 - protein, 13 aa, Artificial Sequence
  Synthetic Construct
FSLSTGGVGVSWI

SEQ ID NO: 4 - protein, 12 aa, Artificial Sequence
  Synthetic Construct
YSITSGYHWGWI

SEQ ID NO: 5 - protein, 11 aa, Artificial Sequence
  Synthetic Construct
FTFTGYWIHWV

SEQ ID NO: 6 - protein, 12 aa, Artificial Sequence
  Synthetic Construct
YSISSGHHWGWI

SEQ ID NO: 7 - protein, 12 aa, Artificial Sequence
  Synthetic Construct
YSITSGYYWGWI

SEQ ID NO: 8 - protein, 11 aa, Artificial Sequence
  Synthetic Construct
YTFTGYAIHWV

SEQ ID NO: 9 - protein, 12 aa, Artificial Sequence
  Synthetic Construct
YSISSGYHWNWI

SEQ ID NO: 10 - protein, 12 aa, Artificial Sequence
  Synthetic Construct
YSISSGYYWDWI

SEQ ID NO: 11 - protein, 12 aa, Artificial Sequence
  Synthetic Construct
YSISSGHYWGWI

SEQ ID NO: 12 - protein, 11 aa, Artificial Sequence
  Synthetic Construct
YTFSNYWIHWV

SEQ ID NO: 13 - protein, 13 aa, Artificial Sequence
  Synthetic Construct
FSLSTGGVGVAWI

SEQ ID NO: 14 - protein, 13 aa, Artificial Sequence
  Synthetic Construct
FSLSTSGVAVGWI

SEQ ID NO: 15 - protein, 13 aa, Artificial Sequence
  Synthetic Construct
FSLSTSGVGVGWI

SEQ ID NO: 16 - protein, 13 aa, Artificial Sequence
  Synthetic Construct
FSLSTGGVAVGWI

SEQ ID NO: 17 - protein, 12 aa, Artificial Sequence
  Synthetic Construct
YSISSGYHWAWI

SEQ ID NO: 18 - protein, 13 aa, Artificial Sequence
  Synthetic Construct
FSLSTSGVAVGWI

SEQ ID NO: 19 - protein, 13 aa, Artificial Sequence
  Synthetic Construct
FSLSTSGVGVGWI

SEQ ID NO: 20 - protein, 12 aa, Artificial Sequence
  Synthetic Construct
YSITSGHHWAWI

SEQ ID NO: 21 - protein, 13 aa, Artificial Sequence
  Synthetic Construct
FSLSTGGVAVGWI

SEQ ID NO: 22 - protein, 13 aa, Artificial Sequence
  Synthetic Construct
FSLSTGGVGVGWI

SEQ ID NO: 23 - protein, 13 aa, Artificial Sequence
  Synthetic Construct
FSLSTGGVAVGWI

SEQ ID NO: 24 - protein, 12 aa, Artificial Sequence
  Synthetic Construct
YSITSGHYWAWI

SEQ ID NO: 25 - protein, 12 aa, Artificial Sequence
  Synthetic Construct
YSITSGHYWAWI

SEQ ID NO: 26 - protein, 11 aa, Artificial Sequence
  Synthetic Construct
FTFSSYWIHWV

SEQ ID NO: 27 - protein, 13 aa, Artificial Sequence
  Synthetic Construct
FSLSTSGVGVGWI

SEQ ID NO: 28 - protein, 12 aa, Artificial Sequence
  Synthetic Construct
YSITSGHYWNWI

SEQ ID NO: 29 - protein, 12 aa, Artificial Sequence
  Synthetic Construct
YSISSGHYWTWI

SEQ ID NO: 30 - protein, 13 aa, Artificial Sequence
  Synthetic Construct
FSLSTGGVGVGWI

SEQ ID NO: 31 - protein, 13 aa, Artificial Sequence
  Synthetic Construct
FSLSTSGVGVGWI

SEQ ID NO: 32 - protein, 13 aa, Artificial Sequence
  Synthetic Construct
FSLSTSGVAVAWI

SEQ ID NO: 33 - protein, 13 aa, Artificial Sequence
  Synthetic Construct
FSLSTSGVGVAWI

SEQ ID NO: 34 - protein, 12 aa, Artificial Sequence
  Synthetic Construct
YSISSGYHWAWI

SEQ ID NO: 35 - protein, 13 aa, Artificial Sequence
  Synthetic Construct
FSLSTGGVGVAWI

SEQ ID NO: 36 - protein, 12 aa, Artificial Sequence
  Synthetic Construct
YSITSGYHWSWI

SEQ ID NO: 37 - protein, 12 aa, Artificial Sequence
  Synthetic Construct
YSISSGHHWAWI

SEQ ID NO: 38 - protein, 13 aa, Artificial Sequence
  Synthetic Construct
FSLSTGGVAVGWI

SEQ ID NO: 39 - protein, 13 aa, Artificial Sequence
  Synthetic Construct
FSLSTSGVGVAWI

SEQ ID NO: 40 - protein, 13 aa, Artificial Sequence
  Synthetic Construct
FSLSTGGVGVAWI

SEQ ID NO: 41 - protein, 13 aa, Artificial Sequence
  Synthetic Construct
FSLSTGGVGVGWI

SEQ ID NO: 42 - protein, 13 aa, Artificial Sequence
  Synthetic Construct
FSLSTSGVGVAWI

SEQ ID NO: 43 - protein, 13 aa, Artificial Sequence
  Synthetic Construct
FSLSTGGVGVGWI

SEQ ID NO: 44 - protein, 13 aa, Artificial Sequence
  Synthetic Construct
FSLSTGGVGVGWI

SEQ ID NO: 45 - protein, 13 aa, Artificial Sequence
  Synthetic Construct
FSLSTGGVGVGWI

SEQ ID NO: 46 - protein, 13 aa, Artificial Sequence
  Synthetic Construct
FSLSTSGVGVGWI

SEQ ID NO: 47 - protein, 13 aa, Artificial Sequence
  Synthetic Construct
FSLSTGGVGVAWI

SEQ ID NO: 48 - protein, 13 aa, Artificial Sequence
  Synthetic Construct
FSLSTSGMGVGWI

SEQ ID NO: 49 - protein, 12 aa, Artificial Sequence
  Synthetic Construct
YSISSGHHWDWI

SEQ ID NO: 50 - protein, 13 aa, Artificial Sequence
  Synthetic Construct
FSLSTSGVGVGWI

SEQ ID NO: 51 - protein, 13 aa, Artificial Sequence
  Synthetic Construct
FSLSTGGVGVGWI

SEQ ID NO: 52 - protein, 13 aa, Artificial Sequence
  Synthetic Construct
FSLSTSGVGVSWI

SEQ ID NO: 53 - protein, 13 aa, Artificial Sequence
  Synthetic Construct
FSLSTSGVGVGWI

SEQ ID NO: 54 - protein, 13 aa, Artificial Sequence
  Synthetic Construct
FSLSTGGVGVAWI

SEQ ID NO: 55 - protein, 13 aa, Artificial Sequence
  Synthetic Construct
FSLSTGGVGVGWI

SEQ ID NO: 56 - protein, 13 aa, Artificial Sequence
  Synthetic Construct
FSLSTSGVGVAWI

SEQ ID NO: 57 - protein, 13 aa, Artificial Sequence
  Synthetic Construct
FSLSTGGVGVGWI

SEQ ID NO: 58 - protein, 13 aa, Artificial Sequence
  Synthetic Construct
FSLSTSGVGVGWI

SEQ ID NO: 59 - protein, 13 aa, Artificial Sequence
  Synthetic Construct
FSLSTSGVGVTWI

SEQ ID NO: 60 - protein, 13 aa, Artificial Sequence
  Synthetic Construct
FSLSTSGVAVAWI

SEQ ID NO: 61 - protein, 21 aa, Artificial Sequence
  Synthetic Construct
VSGISGAGDTTYYADSVKGRF

SEQ ID NO: 62 - protein, 21 aa, Artificial Sequence
  Synthetic Construct
IGIINPNFGDTNYAQKFQGRV

SEQ ID NO: 63 - protein, 21 aa, Artificial Sequence
  Synthetic Construct
VSSISGSGGTTYYADSVKGRF

SEQ ID NO: 64 - protein, 21 aa, Artificial Sequence
  Synthetic Construct
VSAISGAGGSTYYADSVKGRF

SEQ ID NO: 65 - protein, 23 aa, Artificial Sequence
  Synthetic Construct
VGRIRSKTDGYTTEYAAPVKGRF

SEQ ID NO: 66 - protein, 21 aa, Artificial Sequence
  Synthetic Construct
VSGISGYGDTTYYADSVKGRF

SEQ ID NO: 67 - protein, 21 aa, Artificial Sequence
  Synthetic Construct
VSGISGSGSSTYYADSVKGRF

SEQ ID NO: 68 - protein, 21 aa, Artificial Sequence
  Synthetic Construct
VSAISGDGGSTYYADSVKGRF

SEQ ID NO: 69 - protein, 21 aa, Artificial Sequence
  Synthetic Construct
VSSISGSGGSTYYADSVKGRF

SEQ ID NO: 70 - protein, 21 aa, Artificial Sequence
  Synthetic Construct
VSSISGDGDTTYYADSVKGRF

SEQ ID NO: 71 - protein, 20 aa, Artificial Sequence
  Synthetic Construct
LALIDWYGDKYYSTSLKSRL

SEQ ID NO: 72 - protein, 21 aa, Artificial Sequence
  Synthetic Construct
VSYISGDGDTTYYADSVKGRF

SEQ ID NO: 73 - protein, 21 aa, Artificial Sequence
  Synthetic Construct
VSSISGYGSTTYYADSVKGRF

SEQ ID NO: 74 - protein, 20 aa, Artificial Sequence
  Synthetic Construct
LALIDWADDKYYSPSLKSRL

SEQ ID NO: 75 - protein, 20 aa, Artificial Sequence
  Synthetic Construct
LALIDWDDDKYYSPSLKSRL

SEQ ID NO: 76 - protein, 20 aa, Artificial Sequence
  Synthetic Construct
LALIDWAGDKSYSTSLKSRL

SEQ ID NO: 77 - protein, 21 aa, Artificial Sequence
  Synthetic Construct
VSSISGDGSSTYYADSVKGRF

SEQ ID NO: 78 - protein, 20 aa, Artificial Sequence
  Synthetic Construct
LALIDWDDDKYYSTSLKSRL

SEQ ID NO: 79 - protein, 20 aa, Artificial Sequence
  Synthetic Construct
IGEIYHSGSTYYSPSLKSRV

SEQ ID NO: 80 - protein, 21 aa, Artificial Sequence
  Synthetic Construct
VSAISGDGSTTYYADSVKGRF

SEQ ID NO: 81 - protein, 20 aa, Artificial Sequence
  Synthetic Construct
LALIDWAGDKSYSTSLKSRL

SEQ ID NO: 82 - protein, 21 aa, Artificial Sequence
  Synthetic Construct
VSGISGYGSTTYYADSVKGRF

SEQ ID NO: 83 - protein, 21 aa, Artificial Sequence
  Synthetic Construct
VSGISGDGGTTYYADSVKGRF

SEQ ID NO: 84 - protein, 21 aa, Artificial Sequence
  Synthetic Construct
VSAISGSGSSTYYADSVKGRF

SEQ ID NO: 85 - protein, 21 aa, Artificial Sequence
  Synthetic Construct
VSSISGYGSTTYYADSVKGRF

SEQ ID NO: 86 - protein, 21 aa, Artificial Sequence
  Synthetic Construct
VSVISGDGDTTYYADSVKGRF

SEQ ID NO: 87 - protein, 20 aa, Artificial Sequence
  Synthetic Construct
LALIDWADDKYYSPSLKSRL

SEQ ID NO: 88 - protein, 21 aa, Artificial Sequence
  Synthetic Construct
VSGISGDGSSTYYADSVKGRF

SEQ ID NO: 89 - protein, 21 aa, Artificial Sequence
  Synthetic Construct
VSAISGSGSSTYYADSVKGRF

SEQ ID NO: 90 - protein, 20 aa, Artificial Sequence
  Synthetic Construct
LALIDWADDKYYSPSLKSRL

SEQ ID NO: 91 - protein, 21 aa, Artificial Sequence
  Synthetic Construct
VSGISGAGDSTYYADSVKGRF

SEQ ID NO: 92 - protein, 20 aa, Artificial Sequence
  Synthetic Construct
LALIDWDDDKRYSTSLKSRL

SEQ ID NO: 93 - protein, 21 aa, Artificial Sequence
  Synthetic Construct
VSYISGDGGSTYYADSVKGRF

SEQ ID NO: 94 - protein, 20 aa, Artificial Sequence
  Synthetic Construct
IGEIYHSGSTYYSPSLKSRV

SEQ ID NO: 95 - protein, 21 aa, Artificial Sequence
  Synthetic Construct
VSSISGYGSTTYYADSVKGRF

SEQ ID NO: 96 - protein, 21 aa, Artificial Sequence
  Synthetic Construct
VSSISGYGDTTYYADSVKGRF

SEQ ID NO: 97 - protein, 21 aa, Artificial Sequence
  Synthetic Construct
VSVISGSGSSTYYADSVKGRF

SEQ ID NO: 98 - protein, 20 aa, Artificial Sequence
  Synthetic Construct
LALIDWDGDKSYSTSLKSRL

SEQ ID NO: 99 - protein, 21 aa, Artificial Sequence
  Synthetic Construct
VSSISGAGGTTYYADSVKGRF

SEQ ID NO: 100 - protein, 21 aa, Artificial Sequence
  Synthetic Construct
VSYISGSGDTTYYADSVKGRF

SEQ ID NO: 101 - protein, 21 aa, Artificial Sequence
  Synthetic Construct
VSYISGDGDTTYYADSVKGRF

SEQ ID NO: 102 - protein, 21 aa, Artificial Sequence
  Synthetic Construct
VSSISGYGGTTYYADSVKGRF

SEQ ID NO: 103 - protein, 21 aa, Artificial Sequence
  Synthetic Construct
VSYISGAGGTTYYADSVKGRF

SEQ ID NO: 104 - protein, 21 aa, Artificial Sequence
  Synthetic Construct
VSYISGAGGTTYYADSVKGRF

SEQ ID NO: 105 - protein, 21 aa, Artificial Sequence
  Synthetic Construct
VSYISGDGDTTYYADSVKGRF

SEQ ID NO: 106 - protein, 21 aa, Artificial Sequence
  Synthetic Construct
VSYISGDGGSTYYADSVKGRF

SEQ ID NO: 107 - protein, 21 aa, Artificial Sequence
  Synthetic Construct
VSYISGYGGTTYYADSVKGRF

SEQ ID NO: 108 - protein, 21 aa, Artificial Sequence
  Synthetic Construct
VSYISGYGGTTYYADSVKGRF

SEQ ID NO: 109 - protein, 21 aa, Artificial Sequence
  Synthetic Construct
VSYISGAGDTTYYADSVKGRF

SEQ ID NO: 110 - protein, 21 aa, Artificial Sequence
  Synthetic Construct
VSYISGDGGSTYYADSVKGRF

SEQ ID NO: 111 - protein, 21 aa, Artificial Sequence
  Synthetic Construct
VSYISGAGSTTYYADSVKGRF

SEQ ID NO: 112 - protein, 21 aa, Artificial Sequence
  Synthetic Construct
VSYISGSGDTTYYADSVKGRF

SEQ ID NO: 113 - protein, 21 aa, Artificial Sequence
  Synthetic Construct
VSYISGDGDTTYYADSVKGRF

SEQ ID NO: 114 - protein, 21 aa, Artificial Sequence
  Synthetic Construct
VSYISGDGGTTYYADSVKGRF

SEQ ID NO: 115 - protein, 21 aa, Artificial Sequence
  Synthetic Construct
VSYISGAGDSTYYADSVKGRF

SEQ ID NO: 116 - protein, 21 aa, Artificial Sequence
  Synthetic Construct
VSSISGYGGTTYYADSVKGRF

SEQ ID NO: 117 - protein, 21 aa, Artificial Sequence
  Synthetic Construct
VSSISGSGGSTYYADSVKGRF

SEQ ID NO: 118 - protein, 20 aa, Artificial Sequence
  Synthetic Construct
IGDIYHSGSTYYNPSLKSRV

SEQ ID NO: 119 - protein, 20 aa, Artificial Sequence
  Synthetic Construct
IGDIYHSGSTYYSPSLKSRV

SEQ ID NO: 120 - protein, 20 aa, Artificial Sequence
  Synthetic Construct
IGDIYHSGNTYYNPSLKSRV

SEQ ID NO: 121 - protein, 10 aa, Artificial Sequence
  Synthetic Construct
ARERDYDFDY

SEQ ID NO: 122 - protein, 14 aa, Artificial Sequence
  Synthetic Construct
ARDEYYGGSYYFDY

SEQ ID NO: 123 - protein, 12 aa, Artificial Sequence
  Synthetic Construct
ARDDLYSWYFDV

SEQ ID NO: 124 - protein, 12 aa, Artificial Sequence
  Synthetic Construct
ARDGYGGSYFDY

SEQ ID NO: 125 - protein, 11 aa, Artificial Sequence
  Synthetic Construct
ARLGGHWYFDV

SEQ ID NO: 126 - protein, 14 aa, Artificial Sequence
  Synthetic Construct
ARDPYSSGSGYFDY

SEQ ID NO: 127 - protein, 9 aa, Artificial Sequence
  Synthetic Construct
ARGTYSFDV

SEQ ID NO: 128 - protein, 10 aa, Artificial Sequence
  Synthetic Construct
ARGYRGYFDY

SEQ ID NO: 129 - protein, 15 aa, Artificial Sequence
  Synthetic Construct
ARDPNYYSSGSYFDY

SEQ ID NO: 130 - protein, 13 aa, Artificial Sequence
  Synthetic Construct
AREYYGYGYALDY

SEQ ID NO: 131 - protein, 12 aa, Artificial Sequence
  Synthetic Construct
ARSDYYGSHFDY

SEQ ID NO: 132 - protein, 15 aa, Artificial Sequence
  Synthetic Construct
AREGSTTVAGDWFAY

SEQ ID NO: 133 - protein, 15 aa, Artificial Sequence
  Synthetic Construct
AREGSDAVLGDWFGY

SEQ ID NO: 134 - protein, 15 aa, Artificial Sequence
  Synthetic Construct
AREGSDAVLGDWFAY

SEQ ID NO: 135 - protein, 15 aa, Artificial Sequence
  Synthetic Construct
ARGGSDTVLGDWFAY

SEQ ID NO: 136 - protein, 15 aa, Artificial Sequence
  Synthetic Construct
ARGGSDTVLGDWFAY

SEQ ID NO: 137 - protein, 15 aa, Artificial Sequence
  Synthetic Construct
AREGSDAVTGDWFAY

SEQ ID NO: 138 - protein, 15 aa, Artificial Sequence
  Synthetic Construct
AREGSTAVAGDWFAY

SEQ ID NO: 139 - protein, 15 aa, Artificial Sequence
  Synthetic Construct
AREGSDVVTGDWFAY

SEQ ID NO: 140 - protein, 15 aa, Artificial Sequence
  Synthetic Construct
AREGSTAVTGDWFAY

SEQ ID NO: 141 - protein, 15 aa, Artificial Sequence
  Synthetic Construct
AREGEDAVTGDWFAY

SEQ ID NO: 142 - protein, 15 aa, Artificial Sequence
  Synthetic Construct
AREGSDAVAGDWFDY

SEQ ID NO: 143 - protein, 10 aa, Artificial Sequence
  Synthetic Construct
TREDYGPHAY

SEQ ID NO: 144 - protein, 15 aa, Artificial Sequence
  Synthetic Construct
ARGGSDTVLGDWFAY

SEQ ID NO: 145 - protein, 15 aa, Artificial Sequence
  Synthetic Construct
ARGGSDAVLGDWFAY

SEQ ID NO: 146 - protein, 13 aa, Artificial Sequence
  Synthetic Construct
ARSRGLVLDAFDY

SEQ ID NO: 147 - protein, 15 aa, Artificial Sequence
  Synthetic Construct
ARGGSDAVLGDWFAY

SEQ ID NO: 148 - protein, 15 aa, Artificial Sequence
  Synthetic Construct
ARGGSDAVLGDWFAY

SEQ ID NO: 149 - protein, 15 aa, Artificial Sequence
  Synthetic Construct
ARGGSDAVLGDWFAY

SEQ ID NO: 150 - protein, 15 aa, Artificial Sequence
  Synthetic Construct
ARGGSDTVIGDWFAY

SEQ ID NO: 151 - protein, 15 aa, Artificial Sequence
  Synthetic Construct
AREGSDTVLGDWFAY

SEQ ID NO: 152 - protein, 15 aa, Artificial Sequence
  Synthetic Construct
AREGSTTVVGDWFDY

SEQ ID NO: 153 - protein, 15 aa, Artificial Sequence
  Synthetic Construct
AREGSDVVAGDWFAY

SEQ ID NO: 154 - protein, 12 aa, Artificial Sequence
  Synthetic Construct
ARSPYYYGVFDY

SEQ ID NO: 155 - protein, 15 aa, Artificial Sequence
  Synthetic Construct
AREGSDAVLGDWFGY

SEQ ID NO: 156 - protein, 15 aa, Artificial Sequence
  Synthetic Construct
AREGSDTVLGDWFAY

SEQ ID NO: 157 - protein, 15 aa, Artificial Sequence
  Synthetic Construct
AREGSDTVLGDWFAY

SEQ ID NO: 158 - protein, 15 aa, Artificial Sequence
  Synthetic Construct
AREGSTAVVGDWFDY

SEQ ID NO: 159 - protein, 15 aa, Artificial Sequence
  Synthetic Construct
ARGGSTAVTGDWFDY

SEQ ID NO: 160 - protein, 15 aa, Artificial Sequence
  Synthetic Construct
AREGSDTVVGDWFAY

SEQ ID NO: 161 - protein, 15 aa, Artificial Sequence
  Synthetic Construct
AREGSDTVAGDWFAY

SEQ ID NO: 162 - protein, 15 aa, Artificial Sequence
  Synthetic Construct
AREGSDTVLGDWFAY

SEQ ID NO: 163 - protein, 15 aa, Artificial Sequence
  Synthetic Construct
AREGSDTVLGDWFAY

SEQ ID NO: 164 - protein, 15 aa, Artificial Sequence
  Synthetic Construct
AREGSDAVLGDWFAY

SEQ ID NO: 165 - protein, 15 aa, Artificial Sequence
  Synthetic Construct
AREGSTAVLGDWFAY

SEQ ID NO: 166 - protein, 15 aa, Artificial Sequence
  Synthetic Construct
AREGSTAVVGDWFAY

SEQ ID NO: 167 - protein, 15 aa, Artificial Sequence
  Synthetic Construct
AREGSTAVLGDWFAY

SEQ ID NO: 168 - protein, 15 aa, Artificial Sequence
  Synthetic Construct
AREGSDVVAGDWFAY

SEQ ID NO: 169 - protein, 15 aa, Artificial Sequence
  Synthetic Construct
AREGSDAVLGDWFAY

SEQ ID NO: 170 - protein, 15 aa, Artificial Sequence
  Synthetic Construct
AREGSDTVLGDWFAY

SEQ ID NO: 171 - protein, 15 aa, Artificial Sequence
  Synthetic Construct
AREGSDTVLGDWFAY

SEQ ID NO: 172 - protein, 15 aa, Artificial Sequence
  Synthetic Construct
AREGSDAVLGDWFAY

SEQ ID NO: 173 - protein, 15 aa, Artificial Sequence
  Synthetic Construct
AREGSTTVLGDWFAY

SEQ ID NO: 174 - protein, 15 aa, Artificial Sequence
  Synthetic Construct
AREGSDTVLGDWFAY

SEQ ID NO: 175 - protein, 15 aa, Artificial Sequence
  Synthetic Construct
AREGSDTVLGDWFAY

SEQ ID NO: 176 - protein, 15 aa, Artificial Sequence
  Synthetic Construct
AREGSDTVLGDWFAY

SEQ ID NO: 177 - protein, 15 aa, Artificial Sequence
  Synthetic Construct
AREGSDTVLGDWFAY

SEQ ID NO: 178 - protein, 15 aa, Artificial Sequence
  Synthetic Construct
AREGSTAVLGDWFAY

SEQ ID NO: 179 - protein, 15 aa, Artificial Sequence
  Synthetic Construct
AREGSDAVAGDWFDY

SEQ ID NO: 180 - protein, 15 aa, Artificial Sequence
  Synthetic Construct
ARGGSDTVVGDWFAY

SEQ ID NO: 181 - protein, 11 aa, Artificial Sequence
  Synthetic Construct
RASQSVSSYLA

SEQ ID NO: 182 - protein, 11 aa, Artificial Sequence
  Synthetic Construct
RASQDVRTAVA

SEQ ID NO: 183 - protein, 11 aa, Artificial Sequence
  Synthetic Construct
RASQGIGSSLA

SEQ ID NO: 184 - protein, 11 aa, Artificial Sequence
  Synthetic Construct
QASQDISTFLA

SEQ ID NO: 185 - protein, 11 aa, Artificial Sequence
  Synthetic Construct
RASQSIGRYLN

SEQ ID NO: 186 - protein, 11 aa, Artificial Sequence
  Synthetic Construct
RASQGISSALG

SEQ ID NO: 187 - protein, 11 aa, Artificial Sequence
  Synthetic Construct
RASQGISSDLA

SEQ ID NO: 188 - protein, 12 aa, Artificial Sequence
  Synthetic Construct
RASESVTSTYLA

SEQ ID NO: 189 - protein, 11 aa, Artificial Sequence
  Synthetic Construct
RASQGIGSFLG

SEQ ID NO: 190 - protein, 11 aa, Artificial Sequence
  Synthetic Construct
RASQGISSYLA

SEQ ID NO: 191 - protein, 15 aa, Artificial Sequence
  Synthetic Construct
RASESVDFDGFSFLA

SEQ ID NO: 192 - protein, 11 aa, Artificial Sequence
  Synthetic Construct
RASQGIGSFLG

SEQ ID NO: 193 - protein, 11 aa, Artificial Sequence
  Synthetic Construct
QASQDITTYLA

SEQ ID NO: 194 - protein, 11 aa, Artificial Sequence
  Synthetic Construct
RASQDISTFLA

SEQ ID NO: 195 - protein, 11 aa, Artificial Sequence
  Synthetic Construct
RASQSVSPYLA

SEQ ID NO: 196 - protein, 11 aa, Artificial Sequence
  Synthetic Construct
RASQGVSPYLA

SEQ ID NO: 197 - protein, 11 aa, Artificial Sequence
  Synthetic Construct
RASQGVGTYLA

SEQ ID NO: 198 - protein, 11 aa, Artificial Sequence
  Synthetic Construct
QASQDIRTFLA

SEQ ID NO: 199 - protein, 11 aa, Artificial Sequence
  Synthetic Construct
RASQGISSVLA

SEQ ID NO: 200 - protein, 10 aa, Artificial Sequence
  Synthetic Construct
SASSRVGYVH

SEQ ID NO: 201 - protein, 11 aa, Artificial Sequence
  Synthetic Construct
QASQDIRTYLA

SEQ ID NO: 202 - protein, 11 aa, Artificial Sequence
  Synthetic Construct
RASQDIRSYLA

SEQ ID NO: 203 - protein, 11 aa, Artificial Sequence
  Synthetic Construct
RASQSITTYLN

SEQ ID NO: 204 - protein, 11 aa, Artificial Sequence
  Synthetic Construct
RASQGISSSLA

SEQ ID NO: 205 - protein, 11 aa, Artificial Sequence
  Synthetic Construct
RASQGIGSFLA

SEQ ID NO: 206 - protein, 12 aa, Artificial Sequence
  Synthetic Construct
RASQSVSGRFLA

SEQ ID NO: 207 - protein, 11 aa, Artificial Sequence
  Synthetic Construct
RASQGIGSYLA

SEQ ID NO: 208 - protein, 11 aa, Artificial Sequence
  Synthetic Construct
RASQSISSYLA

SEQ ID NO: 209 - protein, 11 aa, Artificial Sequence
  Synthetic Construct
RASQGVGSYLA

SEQ ID NO: 210 - protein, 11 aa, Artificial Sequence
  Synthetic Construct
RASQSIGSYLA

SEQ ID NO: 211 - protein, 11 aa, Artificial Sequence
  Synthetic Construct
RASQDIRSYLA

SEQ ID NO: 212 - protein, 11 aa, Artificial Sequence
  Synthetic Construct
RASQGIGTYLA

SEQ ID NO: 213 - protein, 11 aa, Artificial Sequence
  Synthetic Construct
RASQDISSVLA

SEQ ID NO: 214 - protein, 10 aa, Artificial Sequence
  Synthetic Construct
SASSRVGSVY

SEQ ID NO: 215 - protein, 11 aa, Artificial Sequence
  Synthetic Construct
QASQDITTYLA

SEQ ID NO: 216 - protein, 11 aa, Artificial Sequence
  Synthetic Construct
QASQDIRTYLA

SEQ ID NO: 217 - protein, 11 aa, Artificial Sequence
  Synthetic Construct
RASQGISSYLA

SEQ ID NO: 218 - protein, 11 aa, Artificial Sequence
  Synthetic Construct
RASQGISRYLA

SEQ ID NO: 219 - protein, 11 aa, Artificial Sequence
  Synthetic Construct
RASQGISPYLA

SEQ ID NO: 220 - protein, 11 aa, Artificial Sequence
  Synthetic Construct
RASQGISSYLA

SEQ ID NO: 221 - protein, 11 aa, Artificial Sequence
  Synthetic Construct
RASQGISSYLA

SEQ ID NO: 222 - protein, 11 aa, Artificial Sequence
  Synthetic Construct
RASQGISSYLA

SEQ ID NO: 223 - protein, 11 aa, Artificial Sequence
  Synthetic Construct
RASQGISSYLA

SEQ ID NO: 224 - protein, 11 aa, Artificial Sequence
  Synthetic Construct
RASQGISSYLA

SEQ ID NO: 225 - protein, 11 aa, Artificial Sequence
  Synthetic Construct
RASQGISSYLA

SEQ ID NO: 226 - protein, 11 aa, Artificial Sequence
  Synthetic Construct
RASQGISSYLA

SEQ ID NO: 227 - protein, 11 aa, Artificial Sequence
  Synthetic Construct
RASQGISSYLA

SEQ ID NO: 228 - protein, 11 aa, Artificial Sequence
  Synthetic Construct
RASQGISSYLA

SEQ ID NO: 229 - protein, 11 aa, Artificial Sequence
  Synthetic Construct
RASQGISSYLA

SEQ ID NO: 230 - protein, 11 aa, Artificial Sequence
  Synthetic Construct
RASQGISSYLA

SEQ ID NO: 231 - protein, 11 aa, Artificial Sequence
  Synthetic Construct
RASQGISSYLA

SEQ ID NO: 232 - protein, 11 aa, Artificial Sequence
  Synthetic Construct
RASQGISSYLA

SEQ ID NO: 233 - protein, 11 aa, Artificial Sequence
  Synthetic Construct
RASQGISSYLA

SEQ ID NO: 234 - protein, 11 aa, Artificial Sequence
  Synthetic Construct
RASQGISSYLA

SEQ ID NO: 235 - protein, 11 aa, Artificial Sequence
  Synthetic Construct
RASQGVSSYLA

SEQ ID NO: 236 - protein, 11 aa, Artificial Sequence
  Synthetic Construct
RASQGVSSYLA

SEQ ID NO: 237 - protein, 11 aa, Artificial Sequence
  Synthetic Construct
RASQGVSSYLA

SEQ ID NO: 238 - protein, 11 aa, Artificial Sequence
  Synthetic Construct
RASQSISSYLN

SEQ ID NO: 239 - protein, 11 aa, Artificial Sequence
  Synthetic Construct
RASQSISSYLN

SEQ ID NO: 240 - protein, 11 aa, Artificial Sequence
  Synthetic Construct
RASQSISSYLN

SEQ ID NO: 241 - protein, 9 aa, Artificial Sequence
  Synthetic Construct
DASNLETGV

SEQ ID NO: 242 - protein, 9 aa, Artificial Sequence
  Synthetic Construct
DASNLETGV

SEQ ID NO: 243 - protein, 9 aa, Artificial Sequence
  Synthetic Construct
AASSLQSGV

SEQ ID NO: 244 - protein, 9 aa, Artificial Sequence
  Synthetic Construct
DASSLESGV

SEQ ID NO: 245 - protein, 9 aa, Artificial Sequence
  Synthetic Construct
DASNLETGV

SEQ ID NO: 246 - protein, 9 aa, Artificial Sequence
  Synthetic Construct
AASSLQSGV

SEQ ID NO: 247 - protein, 9 aa, Artificial Sequence
  Synthetic Construct
AASTLQSGV

SEQ ID NO: 248 - protein, 9 aa, Artificial Sequence
  Synthetic Construct
DASNLETGV

SEQ ID NO: 249 - protein, 9 aa, Artificial Sequence
  Synthetic Construct
DASNRATGI

SEQ ID NO: 250 - protein, 9 aa, Artificial Sequence
  Synthetic Construct
DASSLESGV

SEQ ID NO: 251 - protein, 9 aa, Artificial Sequence
  Synthetic Construct
DASNLETGV

SEQ ID NO: 252 - protein, 9 aa, Artificial Sequence
  Synthetic Construct
DASSLESGV

SEQ ID NO: 253 - protein, 9 aa, Artificial Sequence
  Synthetic Construct
DASNRATGI

SEQ ID NO: 254 - protein, 9 aa, Artificial Sequence
  Synthetic Construct
DASSLESGV

SEQ ID NO: 255 - protein, 9 aa, Artificial Sequence
  Synthetic Construct
DASSLESGV

SEQ ID NO: 256 - protein, 9 aa, Artificial Sequence
  Synthetic Construct
DASNRATGI

SEQ ID NO: 257 - protein, 9 aa, Artificial Sequence
  Synthetic Construct
DASSLESGV

SEQ ID NO: 258 - protein, 9 aa, Artificial Sequence
  Synthetic Construct
DASNRATGI

SEQ ID NO: 259 - protein, 9 aa, Artificial Sequence
  Synthetic Construct
DASSLESGV

SEQ ID NO: 260 - protein, 9 aa, Artificial Sequence
  Synthetic Construct
DASSLESGV

SEQ ID NO: 261 - protein, 9 aa, Artificial Sequence
  Synthetic Construct
DASNRATGI

SEQ ID NO: 262 - protein, 9 aa, Artificial Sequence
  Synthetic Construct
DASNRATGI

SEQ ID NO: 263 - protein, 9 aa, Artificial Sequence
  Synthetic Construct
DASNLETGV

SEQ ID NO: 264 - protein, 9 aa, Artificial Sequence
  Synthetic Construct
DASNLETGV

SEQ ID NO: 265 - protein, 9 aa, Artificial Sequence
  Synthetic Construct
DASNLETGV

SEQ ID NO: 266 - protein, 9 aa, Artificial Sequence
  Synthetic Construct
DASSLESGV

SEQ ID NO: 267 - protein, 9 aa, Artificial Sequence
  Synthetic Construct
DASNLETGV

SEQ ID NO: 268 - protein, 9 aa, Artificial Sequence
  Synthetic Construct
DASNLETGV

SEQ ID NO: 269 - protein, 9 aa, Artificial Sequence
  Synthetic Construct
DASNLETGV

SEQ ID NO: 270 - protein, 9 aa, Artificial Sequence
  Synthetic Construct
DASNLETGV

SEQ ID NO: 271 - protein, 9 aa, Artificial Sequence
  Synthetic Construct
DASNRATGI

SEQ ID NO: 272 - protein, 9 aa, Artificial Sequence
  Synthetic Construct
DASNRATGI

SEQ ID NO: 273 - protein, 9 aa, Artificial Sequence
  Synthetic Construct
DASSLESGV

SEQ ID NO: 274 - protein, 9 aa, Artificial Sequence
  Synthetic Construct
DASNLETGV

SEQ ID NO: 275 - protein, 9 aa, Artificial Sequence
  Synthetic Construct
DASNRATGI

SEQ ID NO: 276 - protein, 9 aa, Artificial Sequence
  Synthetic Construct
DASNLETGV

SEQ ID NO: 277 - protein, 9 aa, Artificial Sequence
  Synthetic Construct
DASNLETGV

SEQ ID NO: 278 - protein, 9 aa, Artificial Sequence
  Synthetic Construct
DASNRATGI

SEQ ID NO: 279 - protein, 9 aa, Artificial Sequence
  Synthetic Construct
DASNRATGI

SEQ ID NO: 280 - protein, 9 aa, Artificial Sequence
  Synthetic Construct
DASNLETGV

SEQ ID NO: 281 - protein, 9 aa, Artificial Sequence
  Synthetic Construct
DASNLETGV

SEQ ID NO: 282 - protein, 9 aa, Artificial Sequence
  Synthetic Construct
DASNLETGV

SEQ ID NO: 283 - protein, 9 aa, Artificial Sequence
  Synthetic Construct
DASNLETGV

SEQ ID NO: 284 - protein, 9 aa, Artificial Sequence
  Synthetic Construct
DASNLETGV

SEQ ID NO: 285 - protein, 9 aa, Artificial Sequence
  Synthetic Construct
DASNLETGV

SEQ ID NO: 286 - protein, 9 aa, Artificial Sequence
  Synthetic Construct
DASNLETGV

SEQ ID NO: 287 - protein, 9 aa, Artificial Sequence
  Synthetic Construct
DASNLETGV

SEQ ID NO: 288 - protein, 9 aa, Artificial Sequence
  Synthetic Construct
DASNLETGV

SEQ ID NO: 289 - protein, 9 aa, Artificial Sequence
  Synthetic Construct
DASNLETGV

SEQ ID NO: 290 - protein, 9 aa, Artificial Sequence
  Synthetic Construct
DASNLETGV

SEQ ID NO: 291 - protein, 9 aa, Artificial Sequence
  Synthetic Construct
DASNLETGV

SEQ ID NO: 292 - protein, 9 aa, Artificial Sequence
  Synthetic Construct
DASNLETGV

SEQ ID NO: 293 - protein, 9 aa, Artificial Sequence
  Synthetic Construct
DASNLETGV

SEQ ID NO: 294 - protein, 9 aa, Artificial Sequence
  Synthetic Construct
DASNLETGV

SEQ ID NO: 295 - protein, 9 aa, Artificial Sequence
  Synthetic Construct
DASNLETGV

SEQ ID NO: 296 - protein, 9 aa, Artificial Sequence
  Synthetic Construct
DASNLETGV

SEQ ID NO: 297 - protein, 9 aa, Artificial Sequence
  Synthetic Construct
DASNLETGV

SEQ ID NO: 298 - protein, 9 aa, Artificial Sequence
  Synthetic Construct
AASTLQSGV

SEQ ID NO: 299 - protein, 9 aa, Artificial Sequence
  Synthetic Construct
AASTLQSGV

SEQ ID NO: 300 - protein, 9 aa, Artificial Sequence
  Synthetic Construct
AASTLQSGV

SEQ ID NO: 301 - protein, 11 aa, Artificial Sequence
  Synthetic Construct
YCQQSYSTSHT

SEQ ID NO: 302 - protein, 11 aa, Artificial Sequence
  Synthetic Construct
YCQQSYDWPPT

SEQ ID NO: 303 - protein, 10 aa, Artificial Sequence
  Synthetic Construct
YCQQGYYTWT

SEQ ID NO: 304 - protein, 10 aa, Artificial Sequence
  Synthetic Construct
YCQQAYSIWT

SEQ ID NO: 305 - protein, 11 aa, Artificial Sequence
  Synthetic Construct
YCEQPLELPRT

SEQ ID NO: 306 - protein, 10 aa, Artificial Sequence
  Synthetic Construct
YCQQSYYTWT

SEQ ID NO: 307 - protein, 10 aa, Artificial Sequence
  Synthetic Construct
YCQQYYSYIT

SEQ ID NO: 308 - protein, 11 aa, Artificial Sequence
  Synthetic Construct
YCQQYSDWPPT

SEQ ID NO: 309 - protein, 10 aa, Artificial Sequence
  Synthetic Construct
YCQQSYSLWT

SEQ ID NO: 310 - protein, 11 aa, Artificial Sequence
  Synthetic Construct
YCQQYYSTPLT

SEQ ID NO: 311 - protein, 11 aa, Artificial Sequence
  Synthetic Construct
YCQQYDTLPRT

SEQ ID NO: 312 - protein, 10 aa, Artificial Sequence
  Synthetic Construct
YCQQYYSLVT

SEQ ID NO: 313 - protein, 10 aa, Artificial Sequence
  Synthetic Construct
YCQQGYYLWT

SEQ ID NO: 314 - protein, 10 aa, Artificial Sequence
  Synthetic Construct
YCQQAYSIWT

SEQ ID NO: 315 - protein, 10 aa, Artificial Sequence
  Synthetic Construct
YCQQGYSLWT

SEQ ID NO: 316 - protein, 10 aa, Artificial Sequence
  Synthetic Construct
YCQQGYSTWT

SEQ ID NO: 317 - protein, 10 aa, Artificial Sequence
  Synthetic Construct
YCQQGYSLWT

SEQ ID NO: 318 - protein, 10 aa, Artificial Sequence
  Synthetic Construct
YCQQGYSTWT

SEQ ID NO: 319 - protein, 10 aa, Artificial Sequence
  Synthetic Construct
YCQQGYSLWT

SEQ ID NO: 320 - protein, 10 aa, Artificial Sequence
  Synthetic Construct
YCQQGYYTWT

SEQ ID NO: 321 - protein, 10 aa, Artificial Sequence
  Synthetic Construct
YCQQGYSIWT

SEQ ID NO: 322 - protein, 10 aa, Artificial Sequence
  Synthetic Construct
YCQQGYSIWT

SEQ ID NO: 323 - protein, 10 aa, Artificial Sequence
  Synthetic Construct
YCQQSYSYST

SEQ ID NO: 324 - protein, 10 aa, Artificial Sequence
  Synthetic Construct
YCQQGYSTWT

SEQ ID NO: 325 - protein, 10 aa, Artificial Sequence
  Synthetic Construct
YCQQGYYLWT

SEQ ID NO: 326 - protein, 12 aa, Artificial Sequence
  Synthetic Construct
YCQQYDYWPPYT

SEQ ID NO: 327 - protein, 10 aa, Artificial Sequence
  Synthetic Construct
YCQQGYSIWT

SEQ ID NO: 328 - protein, 10 aa, Artificial Sequence
  Synthetic Construct
YCQQGYSTWT

SEQ ID NO: 329 - protein, 10 aa, Artificial Sequence
  Synthetic Construct
YCQQGYQLWT

SEQ ID NO: 330 - protein, 10 aa, Artificial Sequence
  Synthetic Construct
YCQQGYYLWT

SEQ ID NO: 331 - protein, 10 aa, Artificial Sequence
  Synthetic Construct
YCQQGYSIWT

SEQ ID NO: 332 - protein, 10 aa, Artificial Sequence
  Synthetic Construct
YCQQGYSLWT

SEQ ID NO: 333 - protein, 10 aa, Artificial Sequence
  Synthetic Construct
YCQQGYQIWT

SEQ ID NO: 334 - protein, 11 aa, Artificial Sequence
  Synthetic Construct
YCQQYTHDPVT

SEQ ID NO: 335 - protein, 10 aa, Artificial Sequence
  Synthetic Construct
YCQQGYYLWT

SEQ ID NO: 336 - protein, 10 aa, Artificial Sequence
  Synthetic Construct
YCQQGYSIWT

SEQ ID NO: 337 - protein, 10 aa, Artificial Sequence
  Synthetic Construct
YCQQGYSTWT

SEQ ID NO: 338 - protein, 10 aa, Artificial Sequence
  Synthetic Construct
YCQQGYSLWT

SEQ ID NO: 339 - protein, 10 aa, Artificial Sequence
  Synthetic Construct
YCQQGYSLWT

SEQ ID NO: 340 - protein, 11 aa, Artificial Sequence
  Synthetic Construct
YCQQYYTTPLT

SEQ ID NO: 341 - protein, 11 aa, Artificial Sequence
  Synthetic Construct
YCQQYYTTPLT

SEQ ID NO: 342 - protein, 11 aa, Artificial Sequence
  Synthetic Construct
YCQQYYTTPLT

SEQ ID NO: 343 - protein, 11 aa, Artificial Sequence
  Synthetic Construct
YCQQYYTTPLT

SEQ ID NO: 344 - protein, 11 aa, Artificial Sequence
  Synthetic Construct
YCQQYYTTPLT

SEQ ID NO: 345 - protein, 11 aa, Artificial Sequence
  Synthetic Construct
YCQQYYTTPLT

SEQ ID NO: 346 - protein, 11 aa, Artificial Sequence
  Synthetic Construct
YCQQYYTTPLT

SEQ ID NO: 347 - protein, 11 aa, Artificial Sequence
  Synthetic Construct
YCQQYYSTPLT

SEQ ID NO: 348 - protein, 11 aa, Artificial Sequence
  Synthetic Construct
YCQQYYSTPLT

SEQ ID NO: 349 - protein, 11 aa, Artificial Sequence
  Synthetic Construct
YCQQYYSTPLT

SEQ ID NO: 350 - protein, 11 aa, Artificial Sequence
  Synthetic Construct
YCQQYYSTPLT

SEQ ID NO: 351 - protein, 11 aa, Artificial Sequence
  Synthetic Construct
YCQQYYSTPLT

SEQ ID NO: 352 - protein, 11 aa, Artificial Sequence
  Synthetic Construct
YCQQYYSTPLT

SEQ ID NO: 353 - protein, 11 aa, Artificial Sequence
  Synthetic Construct
YCQQYYSTPLT

SEQ ID NO: 354 - protein, 11 aa, Artificial Sequence [sequence omitted]

SEQ ID NO: 355 - protein, 11 aa, Artificial Sequence
  Synthetic Construct
YCQQYYTTPLT

SEQ ID NO: 356 - protein, 11 aa, Artificial Sequence
  Synthetic Construct
YCQQYYTTPLT

SEQ ID NO: 357 - protein, 11 aa, Artificial Sequence
  Synthetic Construct
YCQQYYTTPLT

SEQ ID NO: 358 - protein, 11 aa, Artificial Sequence
  Synthetic Construct
YCQQYYSTPLT

SEQ ID NO: 359 - protein, 11 aa, Artificial Sequence
  Synthetic Construct
YCQQYYSTPLT

SEQ ID NO: 360 - protein, 11 aa, Artificial Sequence
  Synthetic Construct
YCQQYYSTPLT

SEQ ID NO: 361 - protein, 117 aa, Artificial Sequence
  Synthetic Construct
EVQLVESGGGLVQPGGSLRLSCAASGFTFTSYGIHWVRQAPGKGLEWVSGISGAGDTTYYADSVKGRFTISRDNSKNTLYLQLNSLRAEDTAVYYCARERDYDFDYWGQGTLVTVSS

SEQ ID NO: 362 - protein, 123 aa, Artificial Sequence
  Synthetic Construct
EVQLVESGGGLVQPGGSLRLSCAASGFSLSTSGVAVSWIRQAPGKGLEWIGIINPNFGDTNYAQKFQGRVTISRDNSKNTLYLQLNSLRAEDTAVYYCARDEYYGGSYYFDYWGQGTLVTVSS

SEQ ID NO: 363 - protein, 121 aa, Artificial Sequence
  Synthetic Construct
EVQLVESGGGLVQPGGSLRLSCAASGFSLSTGGVGVSWIRQAPGKGLEWVSSISGSGGTTYYADSVKGRFTISRDNSKNTLYLQLNSLRAEDTAVYYCARDDLYSWYFDVWGQGTLVTVSS

SEQ ID NO: 364 - protein, 120 aa, Artificial Sequence
  Synthetic Construct
EVQLVESGGGLVQPGGSLRLSCAASGYSITSGYHWGWIRQAPGKGLEWVSAISGAGGSTYYADSVKGRFTISRDNSKNTLYLQLNSLRAEDTAVYYCARDGYGGSYFDYWGQGTLVTVSS

SEQ ID NO: 365 - protein, 120 aa, Artificial Sequence
  Synthetic Construct
EVQLVESGGGLVQPGGSLRLSCAASGFTFTGYWIHWVRQAPGKGLEWVGRIRSKTDGYTTEYAAPVKGRFTISRDNSKNTLYLQLNSLRAEDTAVYYCARLGGHWYFDVWGQGTLVTVSS

SEQ ID NO: 366 - protein, 122 aa, Artificial Sequence
  Synthetic Construct
EVQLVESGGGLVQPGGSLRLSCAASGYSISSGHHWGWIRQAPGKGLEWVSGISGYGDTTYYADSVKGRFTISRDNSKNTLYLQLNSLRAEDTAVYYCARDPYSSGSGYFDYWGQGTLVTVSS

SEQ ID NO: 367 - protein, 117 aa, Artificial Sequence
  Synthetic Construct
EVQLVESGGGLVQPGGSLRLSCAASGYSITSGYYWGWIRQAPGKGLEWVSGISGSGSSTYYADSVKGRFTISRDNSKNTLYLQLNSLRAEDTAVYYCARGTYSFDVWGQGTLVTVSS

SEQ ID NO: 368 - protein, 117 aa, Artificial Sequence
  Synthetic Construct
EVQLVESGGGLVQPGGSLRLSCAASGYTFTGYAIHWVRQAPGKGLEWVSAISGDGGSTYYADSVKGRFTISRDNSKNTLYLQLNSLRAEDTAVYYCARGYRGYFDYWGQGTLVTVSS

SEQ ID NO: 369 - protein, 123 aa, Artificial Sequence
  Synthetic Construct
EVQLVESGGGLVQPGGSLRLSCAASGYSISSGYHWNWIRQAPGKGLEWVSSISGSGGSTYYADSVKGRFTISRDNSKNTLYLQLNSLRAEDTAVYYCARDPNYYSSGSYFDYWGQGTLVTVSS

SEQ ID NO: 370 - protein, 121 aa, Artificial Sequence
  Synthetic Construct
EVQLVESGGGLVQPGGSLRLSCAASGYSISSGYYWDWIRQAPGKGLEWVSSISGDGDTTYYADSVKGRFTISRDNSKNTLYLQLNSLRAEDTAVYYCAREYYGYGYALDYWGQGTLVTVSS

SEQ ID NO: 371 - protein, 119 aa, Artificial Sequence
  Synthetic Construct
EVQLVESGGGLVQPGGSLRLSCAASGYSISSGHYWGWIRQAPGKGLEWLALIDWYGDKYYSTSLKSRLTISRDNSKNTLYLQLNSLRAEDTAVYYCARSDYYGSHFDYWGQGTLVTVSS

SEQ ID NO: 372 - protein, 122 aa, Artificial Sequence
  Synthetic Construct
EVQLVESGGGLVQPGGSLRLSCAASGYTFSNYWIHWVRQAPGKGLEWVSYISGDGDTTYYADSVKGRFTISRDNSKNTLYLQLNSLRAEDTAVYYCAREGSTTVAGDWFAYWGQGTLVTVSS

SEQ ID NO: 373 - protein, 124 aa, Artificial Sequence
  Synthetic Construct
EVQLVESGGGLVQPGGSLRLSCAASGFSLSTGGVGVAWIRQAPGKGLEWVSSISGYGSTTYYADSVKGRFTISRDNSKNTLYLQLNSLRAEDTAVYYCAREGSDAVLGDWFGYWGQGTLVTVSS

SEQ ID NO: 374 - protein, 123 aa, Artificial Sequence
  Synthetic Construct
EVQLVESGGGLVQPGGSLRLSCAASGFSLSTSGVAVGWIRQAPGKGLEWLALIDWADDKYYSPSLKSRLTISRDNSKNTLYLQLNSLRAEDTAVYYCAREGSDAVLGDWFAYWGQGTLVTVSS

SEQ ID NO: 375 - protein, 123 aa, Artificial Sequence
  Synthetic Construct
EVQLVESGGGLVQPGGSLRLSCAASGFSLSTSGVGVGWIRQAPGKGLEWLALIDWDDDKYYSPSLKSRLTISRDNSKNTLYLQLNSLRAEDTAVYYCARGGSDTVLGDWFAYWGQGTLVTVSS

SEQ ID NO: 376 - protein, 123 aa, Artificial Sequence
  Synthetic Construct
EVQLVESGGGLVQPGGSLRLSCAASGFSLSTGGVAVGWIRQAPGKGLEWLALIDWAGDKSYSTSLKSRLTISRDNSKNTLYLQLNSLRAEDTAVYYCARGGSDTVLGDWFAYWGQGTLVTVSS

SEQ ID NO: 377 - protein, 123 aa, Artificial Sequence
  Synthetic Construct
EVQLVESGGGLVQPGGSLRLSCAASGYSISSGYHWAWIRQAPGKGLEWVSSISGDGSSTYYADSVKGRFTISRDNSKNTLYLQLNSLRAEDTAVYYCAREGSDAVTGDWFAYWGQGTLVTVSS

SEQ ID NO: 378 - protein, 123 aa, Artificial Sequence
  Synthetic Construct
EVQLVESGGGLVQPGGSLRLSCAASGFSLSTSGVAVGWIRQAPGKGLEWLALIDWDDDKYYSTSLKSRLTISRDNSKNTLYLQLNSLRAEDTAVYYCAREGSTAVAGDWFAYWGQGTLVTVSS

SEQ ID NO: 379 - protein, 123 aa, Artificial Sequence
  Synthetic Construct
EVQLVESGGGLVQPGGSLRLSCAASGFSLSTSGVGVGWIRQAPGKGLEWIGEIYHSGSTYYSPSLKSRVTISRDNSKNTLYLQLNSLKAEDTAVYYCAREGSDVVTGDWFAYWGQGTLVTVSS

SEQ ID NO: 380 - protein, 123 aa, Artificial Sequence
  Synthetic Construct
EVQLVESGGGLVQPGGSLRLSCAASGYSITSGHHWAWIRQAPGKGLEWVSAISGDGSTTYYADSVKGRFTISRDNSKNTLYLQLNSLRAEDTAVYYCAREGSTAVTGDWFAYWGQGTLVTVSS

SEQ ID NO: 381 - protein, 123 aa, Artificial Sequence
  Synthetic Construct
EVQLVESGGGLVQPGGSLRLSCAASGFSLSTGGVAVGWIRQAPGKGLEWLALIDWAGDKSYSTSLKSRLTISRDNSKNTLYLQLNSLRAEDTAVYYCAREGEDAVTGDWFAYWGQGTLVTVSS

SEQ ID NO: 382 - protein, 124 aa, Artificial Sequence
  Synthetic Construct
EVQLVESGGGLVQPGGSLRLSCAASGFSLSTGGVGVGWIRQAPGKGLEWVSGISGYGSTTYYADSVKGRFTISRDNSKNTLYLQLNSLRAEDTAVYYCAREGSDAVAGDWFDYWGQGTLVTVSS

SEQ ID NO: 383 - protein, 119 aa, Artificial Sequence
  Synthetic Construct
EVQLVESGGGLVQPGGSLRLSCAASGFSLSTGGVAVGWIRQAPGKGLEWVSGISGDGGTTYYADSVKGRFTISRDNSKNTLYLQLNSLRAEDTAVYYCTREDYGPHAYWGQGTLVTVSS

SEQ ID NO: 384 - protein, 123 aa, Artificial Sequence
  Synthetic Construct
EVQLVESGGGLVQPGGSLRLSCAASGYSITSGHYWAWIRQAPGKGLEWVSAISGSGSSTYYADSVKGRFTISRDNSKNTLYLQLNSLRAEDTAVYYCARGGSDTVLGDWFAYWGQGTLVTVSS

SEQ ID NO: 385 - protein, 123 aa, Artificial Sequence
  Synthetic Construct
EVQLVESGGGLVQPGGSLRLSCAASGYSITSGHYWAWIRQAPGKGLEWVSSISGYGSTTYYADSVKGRFTISRDNSKNTLYLQLNSLRAEDTAVYYCARGGSDAVLGDWFAYWGQGTLVTVSS

SEQ ID NO: 386 - protein, 120 aa, Artificial Sequence
  Synthetic Construct
EVQLVESGGGLVQPGGSLRLSCAASGFTFSSYWIHWVRQAPGKGLEWVSVISGDGDTTYYADSVKGRFTISRDNSKNTLYLQLNSLRAEDTAVYYCARSRGLVLDAFDYWGQGTLVTVSS

SEQ ID NO: 387 - protein, 123 aa, Artificial Sequence
  Synthetic Construct
EVQLVESGGGLVQPGGSLRLSCAASGFSLSTSGVGVGWIRQAPGKGLEWLALIDWADDKYYSPSLKSRLTISRDNSKNTLYLQLNSLRAEDTAVYYCARGGSDAVLGDWFAYWGQGTLVTVSS

SEQ ID NO: 388 - protein, 123 aa, Artificial Sequence
  Synthetic Construct
EVQLVESGGGLVQPGGSLRLSCAASGYSITSGHYWNWIRQAPGKGLEWVSGISGDGSSTYYADSVKGRFTISRDNSKNTLYLQLNSLRAEDTAVYYCARGGSDAVLGDWFAYWGQGTLVTVSS

SEQ ID NO: 389 - protein, 123 aa, Artificial Sequence
  Synthetic Construct
EVQLVESGGGLVQPGGSLRLSCAASGYSISSGHYWTWIRQAPGKGLEWVSAISGSGSSTYYADSVKGRFTISRDNSKNTLYLQLNSLRAEDTAVYYCARGGSDAVLGDWFAYWGQGTLVTVSS

SEQ ID NO: 390 - protein, 123 aa, Artificial Sequence
  Synthetic Construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSS

SEQ ID NO: 391 - protein, 124 aa, Artificial Sequence
  Synthetic Construct
EVQLVESGGGLVQPGGSLRLSCAASGFSLSTSGVGVGWIRQAPGKGLEWVSGISGAGDSTYYADSVKGRFTISRDNSKNTLYLQLNSLRAEDTAVYYCAREGSDTVLGDWFAYWGQGTLVTVSS

SEQ ID NO: 392 - protein, 123 aa, Artificial Sequence
  Synthetic Construct
EVQLVESGGGLVQPGGSLRLSCAASGFSLSTSGVAVAWIRQAPGKGLEWLALIDWDDDKRYSTSLKSRLTISRDNSKNTLYLQLNSLRAEDTAVYYCAREGSTTVVGDWFDYWGQGTLVTVSS

SEQ ID NO: 393 - protein, 124 aa, Artificial Sequence
  Synthetic Construct
EVQLVESGGGLVQPGGSLRLSCAASGFSLSTSGVGVAWIRQAPGKGLEWVSYISGDGGSTYYADSVKGRFTISRDNSKNTLYLQLNSLRAEDTAVYYCAREGSDVVAGDWFAYWGQGTLVTVSS

SEQ ID NO: 394 - protein, 119 aa, Artificial Sequence
  Synthetic Construct
EVQLVESGGGLVQPGGSLRLSCAASGYSISSGYHWAWIRQAPGKGLEWIGEIYHSGSTYYSPSLKSRVTISRDNSKNTLYLQLNSLRAEDTAVYYCARSPYYYGVFDYWGQGTLVTVSS

SEQ ID NO: 395 - protein, 124 aa, Artificial Sequence
  Synthetic Construct
EVQLVESGGGLVQPGASLRLSCAASGFSLSTGGVGVAWIRQAPGKGLEWVSSISGYGSTTYYADSVKGRFTISRDNSKNTLYLQLNSLRAEDTAVYYCAREGSDAVLGDWFGYWGQGTLVTVSS

SEQ ID NO: 396 - protein, 123 aa, Artificial Sequence
  Synthetic Construct
EVQLVESGGGLVQPGGSLRLSCAASGYSITSGYHWSWIRQAPGKGLEWVSSISGYGDTTYYADSVKGRFTISRDNSKNTLYLQLNSLRAEDTAVYYCAREGSDTVLGDWFAYWGQGTLVTVSS

SEQ ID NO: 397 - protein, 123 aa, Artificial Sequence
  Synthetic Construct
EVQLVESGGGLVQPGGSLRLSCAASGYSISSGHHWAWIRQAPGKGLEWVSVISGSGSSTYYADSVKGRFTISRDNSKNTLYLQLNSLRAEDTAVYYCAREGSDTVLGDWFAYWGQGTLVTVSS

SEQ ID NO: 398 - protein, 123 aa, Artificial Sequence
  Synthetic Construct
EVQLVESGGGLVQPGGSLRLSCAASGFSLSTGGVAVGWIRQAPGKGLEWLALIDWDGDKSYSTSLKSRLTISRDNSKNTLYLQLNSLRAEDTAVYYCAREGSTAVVGDWFDYWGQGTLVTVSS

SEQ ID NO: 399 - protein, 124 aa, Artificial Sequence
  Synthetic Construct
EVQLVESGGGLVQPGGSLRLSCAASGFSLSTSGVGVAWIRQAPGKGLEWVSSISGAGGTTYYADSVKGRFTISRDNSKNTLYLQLNSLRAEDTAVYYCARGGSTAVTGDWFDYWGQGTLVTVSS

SEQ ID NO: 400 - protein, 124 aa, Artificial Sequence
  Synthetic Construct
EVQLVESGGGLVQPGGSLRLSCAASGFSLSTGGVGVAWIRQAPGKGLEWVSYISGSGDTTYYADSVKGRFTISRDNSKNTLYLQLNSLRAEDTAVYYCAREGSDTVVGDWFAYWGQGTLVTVSS

SEQ ID NO: 401 - protein, 124 aa, Artificial Sequence
  Synthetic Construct
EVQLVESGGGLVQPGGSLRLSCAASGFSLSTGGVGVGWIRQAPGKGLEWVSYISGDGDTTYYADSVKGRFTISRDNSKNTLYLQLNSLRAEDTAVYYCAREGSDTVAGDWFAYWGQGTLVTVSS

SEQ ID NO: 402 - protein, 124 aa, Artificial Sequence
  Synthetic Construct
EVQLVESGGGLVQPGGSLRLSCAASGFSLSTSGVGVAWIRQAPGKGLEWVSSISGYGGTTYYADSVKGRFTISRDNSKNTLYLQLNSLRAEDTAVYYCAREGSDTVLGDWFAYWGQGTLVTVSS

SEQ ID NO: 403 - protein, 124 aa, Artificial Sequence
  Synthetic Construct
EVQLVESGGGLVQPGGSLRLSCAASGFSLSTGGVGVGWIRQAPGKGLEWVSYISGAGGTTYYADSVKGRFTISRDNSKNTLYLQLNSLRAEDTAVYYCAREGSDTVLGDWFAYWGQGTLVTVSS

SEQ ID NO: 404 - protein, 124 aa, Artificial Sequence
  Synthetic Construct
EVQLVESGGGLVQPGGSLRLSCAASGFSLSTGGVGVGWIRQAPGKGLEWVSYISGAGGTTYYADSVKGRFTISRDNSKNTLYLQLNSLRAEDTAVYYCAREGSDAVLGDWFAYWGQGTLVTVSS

SEQ ID NO: 405 - protein, 124 aa, Artificial Sequence
  Synthetic Construct
EVQLVESGGGLVQPGGSLRLSCAASGFSLSTGGVGVGWIRQAPGKGLEWVSYISGDGDTTYYADSVKGRFTISRDNSKNTLYLQLNSLRAEDTAVYYCAREGSTAVLGDWFAYWGQGTLVTVSS

SEQ ID NO: 406 - protein, 124 aa, Artificial Sequence
  Synthetic Construct
EVQLVESGGGLVQPGGSLRLSCAASGFSLSTSGVGVGWIRQAPGKGLEWVSYISGDGGSTYYADSVKGRFTISRDNSKNTLYLQLNSLRAEDTAVYYCAREGSTAVVGDWFAYWGQGTLVTVSS

SEQ ID NO: 407 - protein, 124 aa, Artificial Sequence
  Synthetic Construct
EVQLVESGGGLVQPGGSLRLSCAASGFSLSTGGVGVAWIRQAPGKGLEWVSYISGYGGTTYYADSVKGRFTISRDNSKNTLYLQLNSLRAEDTAVYYCAREGSTAVLGDWFAYWGQGTLVTVSS

SEQ ID NO: 408 - protein, 124 aa, Artificial Sequence
  Synthetic Construct
EVQLVESGGGLVQPGGSLRLSCAASGFSLSTSGMGVGWIRQAPGKGLEWVSYISGYGGTTYYADSVKGRFTISRDNSKNTLYLQLNSLRAEDTAVYYCAREGSDVVAGDWFAYWGQGTLVTVSS

SEQ ID NO: 409 - protein, 123 aa, Artificial Sequence
  Synthetic Construct
EVQLVESGGGLVQPGGSLRLSCAASGYSISSGHHWDWIRQAPGKGLEWVSYISGAGDTTYYADSVKGRFTISRDNSKNTLYLQLNSLRAEDTAVYYCAREGSDAVLGDWFAYWGQGTLVTVSS

SEQ ID NO: 410 - protein, 124 aa, Artificial Sequence
  Synthetic Construct
EVQLVESGGGLVQPGGSLRLSCAASGFSLSTSGVGVGWIRQAPGKGLEWVSYISGDGGSTYYADSVKGRFTISRDNSKNTLYLQLNSLRAEDTAVYYCAREGSDTVLGDWFAYWGQGTLVTVSS

SEQ ID NO: 411 - protein, 124 aa, Artificial Sequence
  Synthetic Construct
EVQLVESGGGLVQPGGSLRLSCAASGFSLSTGGVGVGWIRQAPGKGLEWVSYISGAGSTTYYADSVKGRFTISRDNSKNTLYLQLNSLRAEDTAVYYCAREGSDTVLGDWFAYWGQGTLVTVSS

SEQ ID NO: 412 - protein, 124 aa, Artificial Sequence
  Synthetic Construct
EVQLVESGGGLVQPGGSLRLSCAASGFSLSTSGVGVSWIRQAPGKGLEWVSYISGSGDTTYYADSVKGRFTISRDNSKNTLYLQLNSLRAEDTAVYYCAREGSDAVLGDWFAYWGQGTLVTVSS

SEQ ID NO: 413 - protein, 124 aa, Artificial Sequence
  Synthetic Construct
EVQLVESGGGLVQPGGSLRLSCAASGFSLSTSGVGVGWIRQAPGKGLEWVSYISGDGDTTYYADSVKGRFTISRDNSKNTLYLQLNSLRAEDTAVYYCAREGSTTVLGDWFAYWGQGTLVTVSS

SEQ ID NO: 414 - protein, 124 aa, Artificial Sequence
  Synthetic Construct
EVQLVESGGGLVQPGGSLRLSCAASGFSLSTGGVGVAWIRQAPGKGLEWVSYISGDGGTTYYADSVKGRFTISRDNSKNTLYLQLNSLRAEDTAVYYCAREGSDTVLGDWFAYWGQGTLVTVSS

SEQ ID NO: 415 - protein, 124 aa, Artificial Sequence
  Synthetic Construct
EVQLVESGGGLVQPGGSLRLSCAASGFSLSTGGVGVGWIRQAPGKGLEWVSYISGAGDSTYYADSVKGRFTISRDNSKNTLYLQLNSLRAEDTAVYYCAREGSDTVLGDWFAYWGQGTLVTVSS

SEQ ID NO: 416 - protein, 124 aa, Artificial Sequence
  Synthetic Construct
EVQLVESGGGLVQPGGSLRLSCAASGFSLSTSGVGVAWIRQAPGKGLEWVSSISGYGGTTYYADSVKGRFTISRDNSKNRLYLQLNSLRAEDTAVYYCAREGSDTVLGDWFAYWGQGTLVTVSS

SEQ ID NO: 417 - protein, 124 aa, Artificial Sequence
  Synthetic Construct
EVQLVESGGGLVQPGGSLRLSCAASGFSLSTGGVGVGWIRQAPGKGLEWVSSISGSGGSTYYADSVKGRFTISRDNSKNTLYLQLNSLRAEDTAVYYCAREGSDTVLGDWFAYWGQGTLVTVSS

SEQ ID NO: 418 - protein, 123 aa, Artificial Sequence
  Synthetic Construct
EVQLVESGGGLVQPGGSLRLSCAASGFSLSTSGVGVGWIRQAPGKGLEWIGDIYHSGSTYYNPSLKSRVTISRDNSKNTLYLQLNSLRAEDTAVYYCAREGSTAVLGDWFAYWGQGTLVTVSS

SEQ ID NO: 419 - protein, 123 aa, Artificial Sequence
  Synthetic Construct
EVQLVESGGGLVQPGGSLRLSCAASGFSLSTSGVGVTWIRQAPGKSLEWIGDIYHSGSTYYSPSLKSRVTISRDNSKNTLYLQLNSLRAEDTAVYYCAREGSDAVAGDWFDYWGQGTLVTVSS

SEQ ID NO: 420 - protein, 123 aa, Artificial Sequence
  Synthetic Construct
EVQLVESGGGLVQPGGSLRLSCAASGFSLSTSGVAVAWIRQAPGKGLEWIGDIYHSGNTYYNPSLKSRVTISRDNSKNTLYLQLNSLRAEDTAVYYCARGGSDTVVGDWFAYWGQGTLVTVSS

SEQ ID NO: 421 - protein, 108 aa, Artificial Sequence
  Synthetic Construct
DIQLTQSPSSLSASVGDRVTITCRASQSVSSYLAWYQQKPGKAPKLLIYDASNLETGVPSRFSGSGSGTDFTLTISSLQPEDFATYYCQQSYSTSHTFGQGTKVEIKR

SEQ ID NO: 422 - protein, 108 aa, Artificial Sequence
  Synthetic Construct
DIQLTQSPSSLSASVGDRVTITCRASQDVRTAVAWYQQKPGKAPKLLIYDASNLETGVPSRFSGSGSGTDFTLTISSLQPEDFATYYCQQSYDWPPTFGQGTKVEIKR

SEQ ID NO: 423 - protein, 107 aa, Artificial Sequence
  Synthetic Construct
DIQLTQSPSSLSASVGDRVTITCRASQGIGSSLAWYQQKPGKAPKLLIYAASSLQSGVPSRFSGSGSGTDFTLTISSLQPEDFATYYCQQGYYTWTFGQGTKVEIKR

SEQ ID NO: 424 - protein, 107 aa, Artificial Sequence
  Synthetic Construct
DIQLTQSPSSLSASVGDRVTITCQASQDISTFLAWYQQKPGKAPKLLIYDASSLESGVPSRFSGSGSGTDFTLTISSLQPEDFATYYCQQAYSIWTFGQGTKVEIKR

SEQ ID NO: 425 - protein, 108 aa, Artificial Sequence
  Synthetic Construct
DIQLTQSPSSLSASVGDRVTITCRASQSIGRYLNWYQQKPGKAPKLLIYDASNLETGVPSRFSGSGSGTDFTLTISSLQPEDFATYYCEQPLELPRTFGQGTKVEIKR

SEQ ID NO: 426 - protein, 107 aa, Artificial Sequence
  Synthetic Construct
DIQLTQSPSSLSASVGDRVTITCRASQGISSALGWYQQKPGKAPKLLIYAASSLQSGVPSRFSGSGSGTDFTLTISSLQPEDFATYYCQQSYYTWTFGQGTKVEIKR

SEQ ID NO: 427 - protein, 107 aa, Artificial Sequence
  Synthetic Construct
DIQLTQSPSSLSASVGDRVTITCRASQGISSDLAWYQQKPGKAPKLLIYAASTLQSGVPSRFSGSGSGTDFTLTISSLQPEDFATYYCQQYYSYITFGQGTKVEIKR

SEQ ID NO: 428 - protein, 109 aa, Artificial Sequence
  Synthetic Construct
DIQLTQSPSSLSASVGDRVTITCRASESVTSTYLAWYQQKPGKAPKLLIYDASNLETGVPSRFSGSGSGTDFTLTISSLQPEDFATYYCQQYSDWPPTFGQGTKVEIKR

SEQ ID NO: 429 - protein, 107 aa, Artificial Sequence
  Synthetic Construct
DIQLTQSPSSLSASVGDRVTITCRASQGIGSFLGWYQQKPGKAPKLLIYDASNRATGIPSRFSGSGSGTDFTLTISSLQPEDFATYYCQQSYSLWTFGQGTKVEIKR

SEQ ID NO: 430 - protein, 108 aa, Artificial Sequence
  Synthetic Construct
DIQLTQSPSSLSASVGDRVTITCRASQGISSYLAWYQQKPGKAPKLLIYDASSLESGVPSRFSGSGSGTDFTLTISSLQPEDFATYYCQQYYSTPLTFGQGTKVEIKR

SEQ ID NO: 431 - protein, 112 aa, Artificial Sequence
  Synthetic Construct
DIQLTQSPSSLSASVGDRVTITCRASESVDFDGFSFLAWYQQKPGKAPKLLIYDASNLETGVPSRFSGSGSGTDFTLTISSLQPEDFATYYCQQYDTLPRTFGQGTKVEIKR

SEQ ID NO: 432 - protein, 107 aa, Artificial Sequence
  Synthetic Construct
DIQLTQSPSSLSASVGDRVTITCRASQGIGSFLGWYQQKPGKAPKLLIYDASSLESGVPSRFSGSGSGTDFTLTISSLQPEDFATYYCQQYYSLVTFGQGTKVEIKR

SEQ ID NO: 433 - protein, 107 aa, Artificial Sequence
  Synthetic Construct
DIQLTQSPSSLSASVGDRVTITCQASQDITTYLAWYQQKPGKAPKLLIYDASNRATGIPSRFSGSGSGTDFTLTISSLQPEDFATYYCQQGYYLWTFGQGTKVEIKR

SEQ ID NO: 434 - protein, 107 aa, Artificial Sequence
  Synthetic Construct
DIQLTQSPSSLSASVGDRVTITCRASQDISTFLAWYQQKPGKAPKLLIYDASSLESGVPSRFSGSGSGTDFTLTISSLQPEDFATYYCQQAYSIWTFGQGTKVEIKR

SEQ ID NO: 435 - protein, 107 aa, Artificial Sequence
  Synthetic Construct
DIQLTQSPSSLSASVGDRVTITCRASQSVSPYLAWYQQKPGKAPKLLIYDASSLESGVPSRFSGSGSGTDFTLTISSLQPEDFATYYCQQGYSLWTFGQGTKVEIKR

SEQ ID NO: 436 - protein, 107 aa, Artificial Sequence
  Synthetic Construct
DIQLTQSPSSLSASVGDRVTITCRASQGVSPYLAWYQQKPGKAPKLLIYDASNRATGIPSRFSGSGSGTDFTLTISSLQPEDFATYYCQQGYSTWTFGQGTKVEIKR

SEQ ID NO: 437 - protein, 107 aa, Artificial Sequence
  Synthetic Construct
DIQLTQSPSSLSASVGDRVTITCRASQGVGTYLAWYQQKPGKAPKLLIYDASSLESGVPSRFSGSGSGTDFTLTISSLQPEDFATYYCQQGYSLWTFGQGTKVEIKR

SEQ ID NO: 438 - protein, 107 aa, Artificial Sequence
  Synthetic Construct
DIQLTQSPSSLSASVGDRVTITCQASQDIRTFLAWYQQKPGKAPKLLIYDASNRATGIPSRFSGSGSGTDFTLTISSLQPEDFATYYCQQGYSTWTFGQGTKVEIKR

SEQ ID NO: 439 - protein, 107 aa, Artificial Sequence
  Synthetic Construct
DIQLTQSPSSLSASVGDRVTITCRASQGISSVLAWYQQKPGKAPKLLIYDASSLESGVPSRFSGSGSGTDFTLTISSLQPEDFATYYCQQGYSLWTFGQGTKVEIKR

SEQ ID NO: 440 - protein, 106 aa, Artificial Sequence
  Synthetic Construct
DIQLTQSPSSLSASVGDRVTITCSASSRVGYVHWYQQKPGKAPKLLIYDASSLESGVPSRFSGSGSGTDFTLTISSLQPEDFATYYCQQGYYTWTFGQGTKVEIKR

SEQ ID NO: 441 - protein, 107 aa, Artificial Sequence
  Synthetic Construct
DIQLTQSPSSLSASVGDRVTITCQASQDIRTYLAWYQQKPGKAPKLLIYDASNRATGIPSRFSGSGSGTDFTLTISSLQPEDFATYYCQQGYSIWTFGQGTKVEIKR

SEQ ID NO: 442 - protein, 107 aa, Artificial Sequence
  Synthetic Construct
DIQLTQSPSSLSASVGDRVTITCRASQDIRSYLAWYQQKPGKAPKLLIYDASNRATGIPSRFSGSGSGTDFTLTISSLQPEDFATYYCQQGYSIWTFGQGTKVEIKR

SEQ ID NO: 443 - protein, 107 aa, Artificial Sequence
  Synthetic Construct
DIQLTQSPSSLSASVGDRVTITCRASQSITTYLNWYQQKPGKAPKLLIYDASNLETGVPSRFSGSGSGTDFTLTISSLQPEDFATYYCQQSYSYSTFGQGTKVEIKR

SEQ ID NO: 444 - protein, 107 aa, Artificial Sequence
  Synthetic Construct
DIQLTQSPSSLSASVGDRVTITCRASQGISSSLAWYQQKPGKAPKLLIYDASNLETGVPSRFSGSGSGTDFTLTISSLQPEDFATYYCQQGYSTWTFGQGTKVEIKR

SEQ ID NO: 445 - protein, 107 aa, Artificial Sequence
  Synthetic Construct
DIQLTQSPSSLSASVGDRVTITCRASQGIGSFLAWYQQKPGKAPKLLIYDASNLETGVPSRFSGSGSGTDFTLTISSLQPEDFATYYCQQGYYLWTFGQGTKVEIKR

SEQ ID NO: 446 - protein, 110 aa, Artificial Sequence
  Synthetic Construct
DIQLTQSPSSLSASVGDRVTITCRASQSVSGRFLAWYQQKPGKAPKLLIYDASSLESGVPSRFSGSGSGTDFTLTISSLQPEDFATYYCQQYDYWPPYTFGQGTKVEIKR

SEQ ID NO: 447 - protein, 107 aa, Artificial Sequence
  Synthetic Construct
DIQLTQSPSSLSASVGDRVTITCRASQGIGSYLAWYQQKPGKAPKLLIYDASNLETGVPSRFSGSGSGTDFTLTISSLQPEDFATYYCQQGYSIWTFGQGTKVEIKR

SEQ ID NO: 448 - protein, 107 aa, Artificial Sequence
  Synthetic Construct
DIQLTQSPSSLSASVGDRVTITCRASQSISSYLAWYQQKPGKAPKLLIYDASNLETGVPSRFSGSGSGTDFTLTISSLQPEDFATYYCQQGYSTWTFGQGTKVEIKR

SEQ ID NO: 449 - protein, 107 aa, Artificial Sequence
  Synthetic Construct
DIQLTQSPSSLSASVGDRVTITCRASQGVGSYLAWYQQKPGKAPKLLIYDASNLETGVPSRFSGSGSGTDFTLTISSLQPEDFATYYCQQGYQLWTFGQGTKVEIKR

SEQ ID NO: 450 - protein, 107 aa, Artificial Sequence
  Synthetic Construct
DIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 451 - protein, 107 aa, Artificial Sequence; identical to SEQ ID NO: 442
  Synthetic Construct

SEQ ID NO: 452 - protein, 107 aa, Artificial Sequence
  Synthetic Construct
DIQLTQSPSSLSASVGDRVTITCRASQGIGTYLAWYQQKPGKAPKLLIYDASNRATGIPSRFSGSGSGTDFTLTISSLQPEDFATYYCQQGYSLWTFGQGTKVEIKR

SEQ ID NO: 453 - protein, 107 aa, Artificial Sequence
  Synthetic Construct
DIQLTQSPSSLSASVGDRVTITCRASQDISSVLAWYQQKPGKAPKLLIYDASSLESGVPSRFSGSGSGTDFTLTISSLQPEDFATYYCQQGYQIWTFGQGTKVEIKR

SEQ ID NO: 454 - protein, 107 aa, Artificial Sequence
  Synthetic Construct
DIQLTQSPSSLSASVGDRVTITCSASSRVGSVYWYQQKPGKAPKLLIYDASNLETGVPSRFSGSGSGTDFTLTISSLQPEDFATYYCQQYTHDPVTFGQGTKVEIKR

SEQ ID NO: 455 - protein, 107 aa, Artificial Sequence; identical to SEQ ID NO: 433
  Synthetic Construct

SEQ ID NO: 456 - protein, 107 aa, Artificial Sequence
  Synthetic Construct
DIQLTQSPSSLSASVGDRVTITCQASQDIRTYLAWYQQKPGKAPKLLIYDASNLETGVPSRFSGSGSGTDFTLTISSLQPEDFATYYCQQGYSIWTFGQGTKVEIKR

SEQ ID NO: 457 - protein, 107 aa, Artificial Sequence
  Synthetic Construct
DIQLTQSPSSLSASVGDRVTITCRASQGISSYLAWYQQKPGKAPKLLIYDASNLETGVPSRFSGSGSGTDFTLTISSLQPEDFATYYCQQGYSTWTFGQGTKVEIKR

SEQ ID NO: 458 - protein, 107 aa, Artificial Sequence
  Synthetic Construct
DIQLTQSPSSLSASVGDRVTITCRASQGISRYLAWYQQKPGKAPKLLIYDASNRATGIPSRFSGSGSGTDFTLTISSLQPEDFATYYCQQGYSLWTFGQGTKVEIKR

SEQ ID NO: 459 - protein, 107 aa, Artificial Sequence
  Synthetic Construct
DIQLTQSPSSLSASVGDRVTITCRASQGISPYLAWYQQKPGKAPKLLIYDASNRATGIPSRFSGSGSGTDFTLTISSLQPEDFATYYCQQGYSLWTFGQGTKVEIKR

SEQ ID NO: 460 - protein, 108 aa, Artificial Sequence
  Synthetic Construct
DIQLTQSPSSLSASVGDRVTITCRASQGISSYLAWYQQKPGKAPKLLIYDASNLETGVPSRFSGSGSGTDFTLTISSLQPEDFATYYCQQYYTTPLTFGQGTKVEIKR

SEQ ID NO: 461 - protein, 108 aa, Artificial Sequence; identical to SEQ ID NO: 460
  Synthetic Construct

SEQ ID NO: 462 - protein, 108 aa, Artificial Sequence; identical to SEQ ID NO: 460
  Synthetic Construct

SEQ ID NO: 463 - protein, 108 aa, Artificial Sequence; identical to SEQ ID NO: 460
  Synthetic Construct

SEQ ID NO: 464 - protein, 108 aa, Artificial Sequence; identical to SEQ ID NO: 460
  Synthetic Construct

SEQ ID NO: 465 - protein, 108 aa, Artificial Sequence; identical to SEQ ID NO: 460
  Synthetic Construct

SEQ ID NO: 466 - protein, 108 aa, Artificial Sequence; identical to SEQ ID NO: 460
  Synthetic Construct

SEQ ID NO: 467 - protein, 108 aa, Artificial Sequence
  Synthetic Construct
DIQLTQSPSSLSASVGDRVTITCRASQGISSYLAWYQQKPGKAPKLLIYDASNLETGVPSRFSGSGSGTDFTLTISSLQPEDFATYYCQQYYSTPLTFGQGTKVEIKR

SEQ ID NO: 468 - protein, 108 aa, Artificial Sequence; identical to SEQ ID NO: 467
  Synthetic Construct

SEQ ID NO: 469 - protein, 108 aa, Artificial Sequence; identical to SEQ ID NO: 467
  Synthetic Construct

SEQ ID NO: 470 - protein, 108 aa, Artificial Sequence; identical to SEQ ID NO: 467
  Synthetic Construct

SEQ ID NO: 471 - protein, 108 aa, Artificial Sequence; identical to SEQ ID NO: 467
  Synthetic Construct

SEQ ID NO: 472 - protein, 108 aa, Artificial Sequence; identical to SEQ ID NO: 467
  Synthetic Construct

SEQ ID NO: 473 - protein, 108 aa, Artificial Sequence; identical to SEQ ID NO: 467
  Synthetic Construct

SEQ ID NO: 474 - protein, 108 aa, Artificial Sequence
  Synthetic Construct
DIQLTQSPSSLSASVGDRVTITCRASQGISSYLAWYQQKPGKAPKLLIYDASNLETGVPSRFSGSGSGTDFTLTISSLQPEDFATYYCQQSYSTPLTFGQGTKVEIKR

SEQ ID NO: 475 - protein, 108 aa, Artificial Sequence
  Synthetic Construct
DIQLTQSPSSLSASVGDRVTITCRASQGVSSYLAWYQQKPGKAPKLLIYDASNLETGVPSRFSGSGSGTDFTLTISSLQPEDFATYYCQQYYTTPLTFGQGTKVEIKR

SEQ ID NO: 476 - protein, 108 aa, Artificial Sequence; identical to SEQ ID NO: 475
  Synthetic Construct

SEQ ID NO: 477 - protein, 108 aa, Artificial Sequence; identical to SEQ ID NO: 475
  Synthetic Construct

SEQ ID NO: 478 - protein, 108 aa, Artificial Sequence
  Synthetic Construct
DIQLTQSPSSLSASVGDRVTITCRASQSISSYLNWYQQKPGKAPKLLIYAASTLQSGVPSRFSGSGSGTDFTLTISSLQPEDFATYYCQQYYSTPLTFGQGTKVEIKR

SEQ ID NO: 479 - protein, 108 aa, Artificial Sequence; identical to SEQ ID NO: 478
  Synthetic Construct

SEQ ID NO: 480 - protein, 108 aa, Artificial Sequence; identical to SEQ ID NO: 478
  Synthetic Construct

SEQ ID NO: 481 - protein, 450 aa, Artificial Sequence
  Synthetic Construct
EVQLVESGGGLVQPGGSLRLSCAASGFSLSTSGVAVSWIRQAPGKGLEWIGIINPNFGDTNYAQKFQGRVTISRDNSKNTLYLQLNSLRAEDTAVYYCARDEYYGGSYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 482 - protein, 450 aa, Artificial Sequence
  Synthetic Construct
EVQLVESGGGLVQPGGSLRLSCAASGFSLSTSGVAVGWIRQAPGKGLEWLALIDWADDKYYSPSLKSRLTISRDNSKNTLYLQLNSLRAEDTAVYYCAREGSDAVLGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 483 - protein, 450 aa, Artificial Sequence
  Synthetic Construct
EVQLVESGGGLVQPGGSLRLSCAASGFSLSTSGVGVGWIRQAPGKGLEWLALIDWDDDKYYSPSLKSRLTISRDNSKNTLYLQLNSLRAEDTAVYYCARGGSDTVLGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 484 - protein, 450 aa, Artificial Sequence
  Synthetic Construct
EVQLVESGGGLVQPGGSLRLSCAASGYSITSGHYWAWIRQAPGKGLEWVSSISGYGSTTYYADSVKGRFTISRDNSKNTLYLQLNSLRAEDTAVYYCARGGSDAVLGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 485 - protein, 450 aa, Artificial Sequence
  Synthetic Construct
EVQLVESGGGLVQPGGSLRLSCAASGFSLSTSGVGVGWIRQAPGKGLEWLALIDWADDKYYSPSLKSRLTISRDNSKNTLYLQLNSLRAEDTAVYYCARGGSDAVLGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 486 - protein, 450 aa, Artificial Sequence
  Synthetic Construct
EVQLVESGGGLVQPGGSLRLSCAASGFSLSTSGVAVAWIRQAPGKGLEWLALIDWDDDKRYSTSLKSRLTISRDNSKNTLYLQLNSLRAEDTAVYYCAREGSTTVVGDWFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 487 - protein, 446 aa, Artificial Sequence
  Synthetic Construct
EVQLVESGGGLVQPGGSLRLSCAASGYSISSGYHWAWIRQAPGKGLEWIGEIYHSGSTYYSPSLKSRVTISRDNSKNTLYLQLNSLRAEDTAVYYCARSPYYYGVFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 488 - protein, 450 aa, Artificial Sequence
  Synthetic Construct
EVQLVESGGGLVQPGGSLRLSCAASGFSLSTGGVAVGWIRQAPGKGLEWLALIDWDGDKSYSTSLKSRLTISRDNSKNTLYLQLNSLRAEDTAVYYCAREGSTAVVGDWFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 489 - protein, 448 aa, Artificial Sequence
  Synthetic Construct
EVQLVESGGGLVQPGGSLRLSCAASGFSLSTGGVGVSWIRQAPGKGLEWVSSISGSGGTTYYADSVKGRFTISRDNSKNTLYLQLNSLRAEDTAVYYCARDDLYSWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 490 - protein, 448 aa, Artificial Sequence
  Synthetic Construct
EVQLVESGGGLVQPGGSLRLSCAASGYSISSGYYWDWIRQAPGKGLEWVSSISGDGDTTYYADSVKGRFTISRDNSKNTLYLQLNSLRAEDTAVYYCAREYYGYGYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 491 - protein, 446 aa, Artificial Sequence
  Synthetic Construct
EVQLVESGGGLVQPGGSLRLSCAASGYSISSGHYWGWIRQAPGKGLEWLALIDWYGDKYYSTSLKSRLTISRDNSKNTLYLQLNSLRAEDTAVYYCARSDYYGSHFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 492 - protein, 449 aa, Artificial Sequence
  Synthetic Construct
EVQLVESGGGLVQPGGSLRLSCAASGYTFSNYWIHWVRQAPGKGLEWVSYISGDGDTTYYADSVKGRFTISRDNSKNTLYLQLNSLRAEDTAVYYCAREGSTTVAGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 493 - protein, 450 aa, Artificial Sequence
  Synthetic Construct
EVQLVESGGGLVQPGGSLRLSCAASGFSLSTSGVGVGWIRQAPGKGLEWIGEIYHSGSTYYSPSLKSRVTISRDNSKNTLYLQLNSLKAEDTAVYYCAREGSDVVTGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 494 - protein, 450 aa, Artificial Sequence
  Synthetic Construct
EVQLVESGGGLVQPGGSLRLSCAASGYSITSGHYWNWIRQAPGKGLEWVSGISGDGSSTYYADSVKGRFTISRDNSKNTLYLQLNSLRAEDTAVYYCARGGSDAVLGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 495 - protein, 451 aa, Artificial Sequence
  Synthetic Construct
EVQLVESGGGLVQPGGSLRLSCAASGFSLSTSGVGVGWIRQAPGKGLEWVSGISGAGDSTYYADSVKGRFTISRDNSKNTLYLQLNSLRAEDTAVYYCAREGSDTVLGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 496 - protein, 450 aa, Artificial Sequence
  Synthetic Construct
EVQLVESGGGLVQPGGSLRLSCAASGYSISSGHHWAWIRQAPGKGLEWVSVISGSGSSTYYADSVKGRFTISRDNSKNTLYLQLNSLRAEDTAVYYCAREGSDTVLGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 497 - protein, 451 aa, Artificial Sequence
  Synthetic Construct
EVQLVESGGGLVQPGGSLRLSCAASGFSLSTGGVGVAWIRQAPGKGLEWVSSISGYGSTTYYADSVKGRFTISRDNSKNTLYLQLNSLRAEDTAVYYCAREGSDAVLGDWFGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 498 - protein, 450 aa, Artificial Sequence
  Synthetic Construct
EVQLVESGGGLVQPGGSLRLSCAASGFSLSTGGVAVGWIRQAPGKGLEWLALIDWAGDKSYSTSLKSRLTISRDNSKNTLYLQLNSLRAEDTAVYYCARGGSDTVLGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 499 - protein, 450 aa, Artificial Sequence
  Synthetic Construct
EVQLVESGGGLVQPGGSLRLSCAASGYSISSGYHWAWIRQAPGKGLEWVSSISGDGSSTYYADSVKGRFTISRDNSKNTLYLQLNSLRAEDTAVYYCAREGSDAVTGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 500 - protein, 450 aa, Artificial Sequence
  Synthetic Construct
EVQLVESGGGLVQPGGSLRLSCAASGFSLSTSGVAVGWIRQAPGKGLEWLALIDWDDDKYYSTSLKSRLTISRDNSKNTLYLQLNSLRAEDTAVYYCAREGSTAVAGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 501 - protein, 450 aa, Artificial Sequence
  Synthetic Construct
EVQLVESGGGLVQPGGSLRLSCAASGFSLSTGGVAVGWIRQAPGKGLEWLALIDWAGDKSYSTSLKSRLTISRDNSKNTLYLQLNSLRAEDTAVYYCAREGEDAVTGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 502 - protein, 447 aa, Artificial Sequence
  Synthetic Construct
EVQLVESGGGLVQPGGSLRLSCAASGFTFSSYWIHWVRQAPGKGLEWVSVISGDGDTTYYADSVKGRFTISRDNSKNTLYLQLNSLRAEDTAVYYCARSRGLVLDAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 503 - protein, 450 aa, Artificial Sequence
  Synthetic Construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 504 - protein, 450 aa, Artificial Sequence
  Synthetic Construct
EVQLVESGGGLVQPGGSLRLSCAASGYSITSGYHWSWIRQAPGKGLEWVSSISGYGDTTYYADSVKGRFTISRDNSKNTLYLQLNSLRAEDTAVYYCAREGSDTVLGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 505 - protein, 214 aa, Artificial Sequence
  Synthetic Construct
DIQLTQSPSSLSASVGDRVTITCRASQDVRTAVAWYQQKPGKAPKLLIYDASNLETGVPSRFSGSGSGTDFTLTISSLQPEDFATYYCQQSYDWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 506 - protein, 213 aa, Artificial Sequence
  Synthetic Construct
DIQLTQSPSSLSASVGDRVTITCRASQDISTFLAWYQQKPGKAPKLLIYDASSLESGVPSRFSGSGSGTDFTLTISSLQPEDFATYYCQQAYSIWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 507 - protein, 213 aa, Artificial Sequence
  Synthetic Construct
DIQLTQSPSSLSASVGDRVTITCRASQSVSPYLAWYQQKPGKAPKLLIYDASSLESGVPSRFSGSGSGTDFTLTISSLQPEDFATYYCQQGYSLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 508 - protein, 213 aa, Artificial Sequence
  Synthetic Construct
DIQLTQSPSSLSASVGDRVTITCRASQGIGSFLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 509 - protein, 213 aa, Artificial Sequence
  Synthetic Construct
DIQLTQSPSSLSASVGDRVTITCRASQGIGSYLAWYQQKPGKAPKLLIYDASNLETGVPSRFSGSGSGTDFTLTISSLQPEDFATYYCQQGYSIWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 510 - protein, 213 aa, Artificial Sequence
  Synthetic Construct
DIQLTQSPSSLSASVGDRVTITCRASQGIGTYLAWYQQKPGKAPKLLIYDASNRATGIPSRFSGSGSGTDFTLTISSLQPEDFATYYCQQGYSLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 511 - protein, 213 aa, Artificial Sequence
  Synthetic Construct
DIQLTQSPSSLSASVGDRVTITCSASSRVGSVYWYQQKPGKAPKLLIYDASNLETGVPSRFSGSGSGTDFTLTISSLQPEDFATYYCQQYTHDPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 512 - protein, 213 aa, Artificial Sequence
  Synthetic Construct
DIQLTQSPSSLSASVGDRVTITCRASQGISRYLAWYQQKPGKAPKLLIYDASNRATGIPSRFSGSGSGTDFTLTISSLQPEDFATYYCQQGYSLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 513 - protein, 213 aa, Artificial Sequence
  Synthetic Construct
DIQLTQSPSSLSASVGDRVTITCRASQGIGSSLAWYQQKPGKAPKLLIYAASSLQSGVPSRFSGSGSGTDFTLTISSLQPEDFATYYCQQGYYTWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 514 - protein, 214 aa, Artificial Sequence
  Synthetic Construct
DIQLTQSPSSLSASVGDRVTITCRASQGISSYLAWYQQKPGKAPKLLIYDASSLESGVPSRFSGSGSGTDFTLTISSLQPEDFATYYCQQYYSTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 515 - protein, 218 aa, Artificial Sequence
  Synthetic Construct
DIQLTQSPSSLSASVGDRVTITCRASESVDFDGFSFLAWYQQKPGKAPKLLIYDASNLETGVPSRFSGSGSGTDFTLTISSLQPEDFATYYCQQYDTLPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 516 - protein, 213 aa, Artificial Sequence
  Synthetic Construct
DIQLTQSPSSLSASVGDRVTITCRASQGIGSFLGWYQQKPGKAPKLLIYDASSLESGVPSRFSGSGSGTDFTLTISSLQPEDFATYYCQQYYSLVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 517 - protein, 213 aa, Artificial Sequence
  Synthetic Construct
DIQLTQSPSSLSASVGDRVTITCRASQGISSVLAWYQQKPGKAPKLLIYDASSLESGVPSRFSGSGSGTDFTLTISSLQPEDFATYYCQQGYSLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 518 - protein, 213 aa, Artificial Sequence
  Synthetic Construct
DIQLTQSPSSLSASVGDRVTITCRASQSISSYLAWYQQKPGKAPKLLIYDASNLETGVPSRFSGSGSGTDFTLTISSLQPEDFATYYCQQGYSTWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 519 - protein, 213 aa, Artificial Sequence
  Synthetic Construct
DIQLTQSPSSLSASVGDRVTITCRASQDIRSYLAWYQQKPGKAPKLLIYDASNRATGIPSRFSGSGSGTDFTLTISSLQPEDFATYYCQQGYSIWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 520 - protein, 213 aa, Artificial Sequence
  Synthetic Construct
DIQLTQSPSSLSASVGDRVTITCRASQGISSYLAWYQQKPGKAPKLLIYDASNLETGVPSRFSGSGSGTDFTLTISSLQPEDFATYYCQQGYSTWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 521 - protein, 213 aa, Artificial Sequence
  Synthetic Construct
DIQLTQSPSSLSASVGDRVTITCQASQDITTYLAWYQQKPGKAPKLLIYDASNRATGIPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 522 - protein, 213 aa, Artificial Sequence
  Synthetic Construct
DIQLTQSPSSLSASVGDRVTITCRASQGVSPYLAWYQQKPGKAPKLLIYDASNRATGIPSRFSGSGSGTDFTLTISSLQPEDFATYYCQQGYSTWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 523 - protein, 213 aa, Artificial Sequence
  Synthetic Construct
DIQLTQSPSSLSASVGDRVTITCRASQGVGTYLAWYQQKPGKAPKLLIYDASSLESGVPSRFSGSGSGTDFTLTISSLQPEDFATYYCQQGYSLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 524 - protein, 213 aa, Artificial Sequence
  Synthetic Construct
DIQLTQSPSSLSASVGDRVTITCQASQDIRTFLAWYQQKPGKAPKLLIYDASNRATGIPSRFSGSGSGTDFTLTISSLQPEDFATYYCQQGYSTWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 525 - protein, 213 aa, Artificial Sequence
  Synthetic Construct
DIQLTQSPSSLSASVGDRVTITCQASQDIRTYLAWYQQKPGKAPKLLIYDASNRATGIPSRFSGSGSGTDFTLTISSLQPEDFATYYCQQGYSIWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 526 - protein, 216 aa, Artificial Sequence
  Synthetic Construct
DIQLTQSPSSLSASVGDRVTITCRASQSVSGRFLAWYQQKPGKAPKLLIYDASSLESGVPSRFSGSGSGTDFTLTISSLQPEDFATYYCQQYDYWPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 527 - protein, 213 aa, Artificial Sequence
  Synthetic Construct
DIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 528 - protein, 213 aa, Artificial Sequence
  Synthetic Construct
DIQLTQSPSSLSASVGDRVTITCQASQDIRTYLAWYQQKPGKAPKLLIYDASNLETGVPSRFSGSGSGTDFTLTISSLQPEDFATYYCQQGYSIWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 529 - DNA, 762 bp, Homo sapiens
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 530 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 531 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 532 - protein, 11 aa, Artificial Sequence
  Synthetic Construct
  VARIANT 1: Xaa = Phe or Tyr
  VARIANT 4: Xaa = Ser or Thr
  VARIANT 5: Xaa = Gly, Asn, or Ser
  VARIANT 7: Xaa = Ala, Gly, or Trp
XTFXXYXIHWV

SEQ ID NO: 533 - protein, 12 aa, Artificial Sequence
  Synthetic Construct
  VARIANT 4: Xaa = Ser or Thr
  VARIANT 7, 8: Xaa = His or Tyr
  VARIANT 10: Xaa = Ala, Asp, Gly, Asn, Ser, or Thr
YSIXSGXXWXWI

SEQ ID NO: 534 - protein, 13 aa, Artificial Sequence
  Synthetic Construct
  VARIANT 6: Xaa = Gly or Ser
  VARIANT 9: Xaa = Ala or Gly
  VARIANT 11: Xaa = Ala, Gly, Ser, or Thr
FSLSTXGVXVXWI

SEQ ID NO: 535 - protein, 20 aa, Artificial Sequence
  Synthetic Construct
  VARIANT 7: Xaa = Ala, Asp, or Tyr
  VARIANT 8: Xaa = Asp or Gly
  VARIANT 11: Xaa = Arg, Ser, or Tyr
  VARIANT 14: Xaa = Pro or Thr
LALIDWXXDKXYSXSLKSRL

SEQ ID NO: 536 - protein, 20 aa, Artificial Sequence
  Synthetic Construct
  VARIANT 3: Xaa = Asp or Glu
  VARIANT 9, 13: Xaa = Asn or Ser
IGXIYHSGXTYYXPSLKSRV

SEQ ID NO: 537 - protein, 21 aa, Artificial Sequence
  Synthetic Construct
  VARIANT 3: Xaa = Ala, Gly, Ser, Val, or Tyr
  VARIANT 7: Xaa = Ala, Asp, Ser, or Tyr
  VARIANT 9: Xaa = Asp, Gly, or Ser
  VARIANT 10: Xaa = Ser or Thr
VSXISGXGXXTYYADSVKGRF

SEQ ID NO: 538 - protein, 15 aa, Artificial Sequence
  Synthetic Construct
  VARIANT 3: Xaa = Glu or Gly
  VARIANT 5: Xaa = Glu or Ser
  VARIANT 6: Xaa = Asp or Thr
  VARIANT 7: Xaa = Ala, Thr, or Val
  VARIANT 9: Xaa = Ala, Ile, Leu, Thr, or Val
  VARIANT 14: Xaa = Ala, Asp, or Gly
ARXGXXXVXGDWFXY

SEQ ID NO: 539 - protein, 11 aa, Artificial Sequence
  Synthetic Construct
  VARIANT 1: Xaa = Gln or Arg
  VARIANT 5: Xaa = Asp, Gly, or Ser
  VARIANT 6: Xaa = Ile or Val
  VARIANT 7: Xaa = Gly, Arg, Ser, or Thr
  VARIANT 8: Xaa = Pro, Arg, Ser, or Thr
  VARIANT 9: Xaa = Ala, Asp, Phe, Ser, Val, or Tyr
  VARIANT 10: Xaa = Leu or Val
  VARIANT 11: Xaa = Ala, Gly, or Asn
XASQXXXXXXX

SEQ ID NO: 540 - protein, 9 aa, Artificial Sequence
  Synthetic Construct
  VARIANT 1: Xaa = Ala or Asp
  VARIANT 4: Xaa = Asn, Ser, or Thr
  VARIANT 5: Xaa = Leu or Arg
  VARIANT 6: Xaa = Ala, Glu, or Gln
  VARIANT 7: Xaa = Ser or Thr
  VARIANT 9: Xaa = Ile or Val
XASXXXXGX

SEQ ID NO: 541 - protein, 10 aa, Artificial Sequence
  Synthetic Construct
  VARIANT 5: Xaa = Ala, Gly, Ser, or Tyr
  VARIANT 7: Xaa = Gln, Ser, or Tyr
  VARIANT 8: Xaa = Ile, Leu, Thr, or Tyr
  VARIANT 9: Xaa = Ile, Ser, Val, or Trp
YCQQXYXXXT

SEQ ID NO: 542 - protein, 11 aa, Artificial Sequence
  Synthetic Construct
  VARIANT 3: Xaa = Glu or Gln
  VARIANT 5: Xaa = Pro, Ser, or Tyr
  VARIANT 6: Xaa = Asp, Leu, Ser, Thr, or Tyr
  VARIANT 7: Xaa = Asp, Glu, His, Ser, or Thr
  VARIANT 8: Xaa = Asp, Leu, Thr, or Trp
  VARIANT 10: Xaa = Leu, Pro, Arg, or Val
YCXQXXXXPXT

SEQ ID NO: 543 - protein, 256 aa, Mus musculus
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 544 - protein, 254 aa, Macaca fascicularis
MGNSCYNIVATLLLVLNFERTRSLQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQIIFFLALTSTVVLFLLFFLVLRFSVVKRSRKKLLYIFKQPFMRPVQTTQEEDGCSCRFPEEEEGGCEL

(19) United States
(12) Patent Application Publication
(10) Pub. No.: US 2020/0377608 A1
(43) Pub. Date: Dec. 3, 2020

(54) METHODS FOR USING CD137 LIGAND AS A BIOMARKER FOR TREATMENT WITH ANTI-CD137 ANTIBODY

(71) Applicants: Peter Peizhi LUO, Suzhou, Jiangsu (GB); Adagene Inc., Grand Cayman (KY)

(72) Inventors: Peter Peizhi LUO, Suzhou, Jiangsu (CN); Guizhong LIU, Suzhou, Jiangsu (CN); Fangyong DU, Suzhou, Jiangsu (CN)

(73) Assignee: Adagene Inc., Grand Cayman (KY)

(21) Appl. No.: 16/768,500

(22) PCT Filed: Nov. 30, 2018
(86) PCT No.: PCT/CN2018/118631

§ 371 Date: May 29, 2020

(30) Foreign Application Priority Data
Dec. 1, 2017 (CN) PCT/CN2017/114247

(51) Int. Cl. C07K 16/28; A61P 35/00

(52) U.S. Cl. CPC C07K 16/2878; A61K 2039/505; A61P 35/00

(57) ABSTRACT

Provided herein are methods relating to the use of CD137 Ligand (CD137L) expression; methods relating to the use of CD137L expression as a biomarker; and methods for predicting, assessing, and/or aiding in assessment of responsiveness of a subject in need thereof to treatment with particular anti-cancer therapies (e.g., an anti-CD137 antibody, a checkpoint blockade immunotherapy).

### CROSS REFERENCE TO RELATED APPLICATIONS

This application claims the priority benefit of International Patent Application Serial No. PCT/CN2017/114247, filed Dec. 1, 2017, which is hereby incorporated by reference in its entirety.

### REFERENCE TO SEQUENCE LISTING

The content of the following submission on ASCII text file is incorporated herein by reference in its entirety: a computer readable form (CRF) of the Sequence Listing (file name: 695402000441SEQLIST.TXT, date recorded: Nov. 28, 2018, size: 367 KB).

### FIELD OF THE INVENTION

The present disclosure relates to CD137 Ligand (CD137L) expression, and to the use of CD137L expression as a biomarker.

### BACKGROUND

Activation of T cells plays a central role in antitumor immunity. Two key signals are required to activate naïve T cells. Signal one is provided through the T-cell receptor (TCR), while signal two is that of co-stimulation. The CD28:B7 molecules are some of the best-studied costimulatory pathways, thought to be the main mechanism through which primary T cell stimulation occurs. However, a number of other molecules have been identified which serve to amplify and diversify the T cell response following initial T cell activation. These include CD137:CD137 ligand (CD137L) molecules, also known as 4-1BB:4-1BB ligand (4-1BBL). CD137:CD137L are members of the Tumor Necrosis Factor (TNF) Receptor (TNFR):TNF ligand family, which are expressed on T cells and antigen-presenting cells (APCs), respectively. Therapies targeting the CD137:CD137L signaling pathway have been shown to have antitumor effects in a number of model systems, and agonistic anti-CD137 antibodies have also entered clinical development (Yonezawa et al. Clin. Cancer Res. 2015 Jul. 15; 21(14):3113-20; Tolcher et al. Clin Cancer Res. 2017 Sep. 15; 23(18):5349-5357). Also, CD137 ligand cross-links its receptor, CD137, which is expressed on activated T cells, and costimulates T cell activities. CD137 ligand can also be expressed as a transmembrane protein on the cell surface and transmit signals into the cells on which it is expressed (reverse signaling) (Herbert Schwarz et al. J. Leukoc. Biol. 89: 21-29; 2011).

Previous studies indicated that CD137L gene delivery into multiple mouse tumor models can enable the host mice to develop long-term immunity against wild-type tumors (Melero et al. Eur. J. Immunol. 1998 March; 28(3):1116-21), prevent tumor formation or induce tumor regression from the transfectants (Guinn et al. J Immunol. 1999 Apr. 15; 162(8):5003-10; Xiang Cancer Biother. Radiopharm. 1999 October; 14(5):353-61), or improve host survival (Martinet et al. J Natl Cancer Inst. 2000 Jun. 7; 92(11):931-6). These results suggest that CD137L expression may modify the tumor cells for whole cell vaccination by improving their ability to act as APCs for their tumor antigens, with the costimulatory CD137L molecules providing an abundance of signal two. However, suitable development of strategies using CD137L expression as a biomarker for informing decisions regarding immunotherapy treatment in cancer patients remains lacking.

All references cited herein, including patent applications, patent publications, and UniProtKB/Swiss-Prot Accession numbers are herein incorporated by reference in their entirety, as if each individual reference were specifically and individually indicated to be incorporated by reference.

### BRIEF SUMMARY

To meet the above and other needs, disclosed herein are methods for using CD137L expression as a biomarker for: 1) determining whether a subject is likely to respond to certain anti-cancer therapies (e.g., anti-CD137 antibody therapy, checkpoint blockade immunotherapy); 2) predicting responsiveness and/or monitoring, assessing or aiding in assessment of treatment/responsiveness of a subject to certain anti-cancer therapies (e.g., anti-CD137 antibody therapy, checkpoint blockade immunotherapy); 3) selecting a subject to receive/not receive certain anti-cancer therapies (e.g., anti-CD137 antibody therapy, checkpoint blockade immunotherapy); 4) treating or delaying progression of cancer in a subject by administering an effective amount of an anti-cancer therapy (e.g., anti-CD137 antibody therapy, checkpoint blockade immunotherapy); 5) negatively stratifying cancer patients for immunotherapy comprising agonistic anti-CD137 antibodies; and/or 6) positively stratifying cancer patients for immunotherapy comprising checkpoint blockade-directed antibodies (e.g., anti-PD-1/anti-PD-L1 antibodies). The present disclosure is based, in part, on the surprising finding that CD137L can be used as a biomarker to stratify patients, where low levels of CD137L is predictive for responsiveness to anti-CD137 antibody therapy, while high levels of CD137L is predictive for non-responsiveness to anti-CD137 antibody therapy and responsiveness to checkpoint blockade immunotherapies (See e.g., FIGS. 4 and 5, as well as Example 3 below).

Accordingly, in one aspect, provided herein are methods of treating or delaying progression of cancer in a subject in need thereof comprising administering an effective amount of an anti-CD137 antibody to the subject if the level of expression of CD137 ligand (CD137L) in a sample obtained from the subject is lower than a reference level. In some embodiments, the method further comprises the steps of a) obtaining the sample from the subject, and b) measuring the level of expression of CD137L in the sample prior to administration of the anti-CD137 antibody to the subject.

In another aspect, provided herein are methods of treating or delaying progression of cancer in a subject in need thereof comprising administering an effective amount of an anti-CD137 antibody to the subject, wherein it has been determined that the subject is likely to respond to the anti-CD137 antibody when the level of expression of CD137L in a sample obtained from the subject is lower than a reference level.

In another aspect, provided herein are methods of determining if a subject is likely to respond to an anti-CD137 antibody comprising a) obtaining a sample from the subject, b) measuring the level of expression of CD137L in the sample, and c) determining that the subject is likely to respond to the anti-CD137 antibody when the level of expression of CD137L in the sample is lower than a reference level.

In another aspect, provided herein are methods of selecting a subject having cancer for treatment with an anti-CD137 antibody comprising a) measuring the level of expression of CD137L in a sample obtained from the subject, and b) selecting the subject for treatment with the anti-CD137 antibody if the level of expression of CD137L in the sample is lower than a reference level.

In some embodiments that may be combined with any of the preceding embodiments, the level of expression of CD137L in the sample is below the limit of detection.

In some embodiments that may be combined with any of the preceding embodiments, the anti-CD137 antibody binds to an extracellular domain of human CD137, wherein the antibody or the antigen-binding fragment thereof binds to one or more amino acid residues within amino acid residues 34-108 of SEQ ID NO: 531. In some embodiments that may be combined with any of the preceding embodiments, the anti-CD137 antibody binds to one or more amino acid residues within amino acid residues 34-93 of SEQ ID NO: 531. In some embodiments that may be combined with any of the preceding embodiments, the anti-CD137 antibody binds to one or more amino acid residues selected from the group consisting of amino acid residues 34-36, 53-55, and 92-93 of SEQ ID NO: 531. In some embodiments, the anti-CD137 antibody binds to one or more of amino acid residues 34-36, one or more of amino acid residues 53-55, and one or more or amino acid residues 92-93 of SEQ ID NO: 531. In some embodiments that may be combined with any of the preceding embodiments, the anti-CD137 antibody does not bind to one or more of amino acid residues selected from the group consisting of amino acid residues 109-112, 125, 126, 135-138, 150 and 151 of SEQ ID NO: 531. In some embodiments, the anti-CD137 antibody does not bind to amino acid residues 109-112, 125, 126, 135-138, 150 and 151 of SEQ ID NO: 531.

In some embodiments that may be combined with any of the preceding embodiments, the anti-CD137 antibody is cross-reactive with a CD137 polypeptide from at least one non-human species selected from the group consisting of cynomolgus monkey, mouse, rat and dog. In some embodiments, the anti-CD137 antibody binds to cynomolgus monkey CD137. In some embodiments that may be combined with any of the preceding embodiments, the anti-CD137 antibody is a human antibody. In some embodiments that may be combined with any of the preceding embodiments, the anti-CD137 antibody comprises a human IgG1 or human IgG4 Fc region. In some embodiments that may be combined with any of the preceding embodiments, the anti-CD137 antibody comprises an Fc region with one or more effector functions (e.g., C1q binding, CDC, Fc receptor binding, ADCC, and/or phagocytosis). In some embodiments that may be combined with any of the preceding embodiments, the anti-CD137 antibody is an anti-CD137 agonist antibody. In some embodiments that may be combined with any of the preceding embodiments, the anti-CD137 antibody blocks binding of CD137L to CD137. In some embodiments that may be combined with any of the preceding embodiments, the anti-CD137 antibody blocks binding of CD137L to CD137 with an IC50 of less than about 100 nM, less than about 50 nM, or less than about 10 nM.

In some embodiments that may be combined with any of the preceding embodiments, the anti-CD137 antibody comprises a heavy chain variable region and a light chain variable region, wherein the heavy chain variable region comprises an HVR-H1 comprising an amino acid sequence selected from the group consisting of SEQ ID NOS: 1-60, an HVR-H2 comprising an amino acid sequence selected from the group consisting of SEQ ID NOS: 61-120, and an HVR-H3 comprising an amino acid sequence selected from the group consisting of SEQ ID NOS: 121-180, and wherein the light chain variable region comprises an HVR-L1 comprising an amino acid sequence selected from the group consisting of SEQ ID NOS: 181-240, an HVR-L2 comprising an amino acid sequence selected from the group consisting of SEQ ID NOS: 241-300, and an HVR-L3 comprising an amino acid sequence selected from the group consisting of SEQ ID NOS: 301-360. In some embodiments, the heavy chain variable region comprises an amino acid sequence selected from the group consisting of SEQ ID NOS: 361-420, and wherein the light chain variable region comprises an amino acid sequence selected from the group consisting of SEQ ID NOS: 421-480. In some embodiments, the anti-CD137 antibody comprises a heavy chain and a light chain, wherein the heavy chain comprises an amino acid sequence selected from the group consisting of SEQ ID NOS: 481-504, and wherein the light chain comprises an amino acid sequence selected from the group consisting of SEQ ID NOS: 505-528.

In some embodiments, the anti-CD137 antibody comprises a heavy chain variable region and a light chain variable region, wherein the heavy chain variable region comprises an HVR-H1 comprising the amino acid sequence of SEQ ID NO: 15, an HVR-H2 comprising the amino acid sequence of SEQ ID NO: 75, and an HVR-H3 comprising the amino acid sequence of SEQ ID NO: 135, and wherein the light chain variable region comprises an HVR-L1 comprising the amino acid sequence of SEQ ID NO: 195, an HVR-L2 comprising the amino acid sequence of SEQ ID NO: 255, and an HVR-L3 comprising the amino acid sequence of SEQ ID NO: 315. In some embodiments, the heavy chain variable region comprises the amino acid sequence of SEQ ID NO: 375, and wherein the light chain variable region comprises the amino acid sequence of SEQ ID NO: 435. In some embodiments, the anti-CD137 antibody comprises a heavy chain and a light chain, wherein the heavy chain comprises the amino acid sequence of SEQ ID NO: 483, and wherein the light chain comprises the amino acid sequence of SEQ ID NO: 507.

In some embodiments, the anti-CD137 antibody comprises a heavy chain variable region and a light chain variable region, wherein the heavy chain variable region comprises an HVR-H1 comprising the amino acid sequence of SEQ ID NO: 25, an HVR-H2 comprising the amino acid sequence of SEQ ID NO: 85, and an HVR-H3 comprising the amino acid sequence of SEQ ID NO: 145, and wherein the light chain variable region comprises an HVR-L1 comprising the amino acid sequence of SEQ ID NO: 205, an HVR-L2 comprising the amino acid sequence of SEQ ID NO: 265, and an HVR-L3 comprising the amino acid sequence of SEQ ID NO: 325. In some embodiments, the heavy chain variable region comprises the amino acid sequence of SEQ ID NO: 385, and wherein the light chain variable region comprises the amino acid sequence of SEQ ID NO: 445. In some embodiments, the anti-CD137 antibody comprises a heavy chain and a light chain, wherein the heavy chain comprises the amino acid sequence of SEQ ID NO: 484, and wherein the light chain comprises the amino acid sequence of SEQ ID NO: 508.

In some embodiments, the anti-CD137 antibody comprises a heavy chain variable region and a light chain variable region, wherein the heavy chain variable region comprises an HVR-H1 comprising the amino acid sequence of SEQ ID NO: 30, an HVR-H2 comprising the amino acid sequence of SEQ ID NO: 90, and an HVR-H3 comprising the amino acid sequence of SEQ ID NO: 150, and wherein the light chain variable region comprises an HVR-L1 comprising the amino acid sequence of SEQ ID NO: 210, an HVR-L2 comprising the amino acid sequence of SEQ ID NO: 270, and an HVR-L3 comprising the amino acid sequence of SEQ ID NO: 330. In some embodiments, the heavy chain variable region comprises the amino acid sequence of SEQ ID NO: 390, and wherein the light chain variable region comprises the amino acid sequence of SEQ ID NO: 450. In some embodiments, the anti-CD137 antibody comprises a heavy chain and a light chain, wherein the heavy chain comprises the amino acid sequence of SEQ ID NO: 503, and wherein the light chain comprises the amino acid sequence of SEQ ID NO: 527.

In some embodiments that may be combined with any of the preceding embodiments, the methods further comprise administering to the subject a therapeutically effective amount of at least one additional therapeutic agent. In some embodiments, the at least one additional therapeutic agent is selected from viral gene therapy, immune checkpoint inhibitors, target therapies, radiation therapies, and chemotherapies. In some embodiments, the at least one additional therapeutic agent is selected from pomalyst, revlimid, lenalidomide, pomalidomide, thalidomide, a DNA-alkylating platinum-containing derivative, cisplatin, 5-fluorouracil, cyclophosphamide, an anti-CTLA4 antibody, an anti-PD-1 antibody, an anti-PD-L1 antibody, an anti-CD20 antibody, an anti-CD40 antibody, an anti-DRS antibody, an anti-CD1d antibody, an anti-TIM3 antibody, an anti-SLAMF7 antibody, an anti-KIR receptor antibody, an anti-OX40 antibody, an anti-HER2 antibody, an anti-ErbB-2 antibody, an anti-EGFR antibody, cetuximab, rituximab, trastuzumab, pembrolizumab, radiotherapy, single dose radiation, fractionated radiation, focal radiation, whole organ radiation, IL-12, IFNα, GM-CSF, a chimeric antigen receptor, adoptively transferred T cells, an anti-cancer vaccine, and an oncolytic virus.

In another aspect, provided herein are methods of treating or delaying progression of cancer in a subject in need thereof comprising administering an effective amount of a checkpoint blockade immunotherapy to the subject if the level of expression of CD137L in a sample obtained from the subject is higher than a reference level. In some embodiments, the method further comprises a) obtaining the sample from the subject, and b) measuring the level of expression of CD137L in the sample prior to administration of the checkpoint blockade immunotherapy to the subject.

In another aspect, provided herein are methods of treating or delaying progression of cancer in a subject in need thereof comprising administering an effective amount of a checkpoint blockade immunotherapy to the subject, wherein it has been determined that the subject is likely to respond to the checkpoint blockade immunotherapy when the level of expression of CD137L in a sample obtained from the subject is higher than a reference level.

In another aspect, provided herein are methods of determining if a subject is likely to respond to a checkpoint blockade immunotherapy comprising a) obtaining a sample from the subject, b) measuring the level of expression of CD137L in the sample, and c) determining that the subject is likely to respond to the checkpoint blockade immunotherapy when the level of expression of CD137L in the sample is higher than a reference level.

In another aspect, provided herein are methods of selecting a subject having cancer for treatment with a checkpoint blockade immunotherapy comprising a) measuring the level of CD137L expression in a sample obtained from the subject, and b) selecting the subject for treatment with the checkpoint blockade immunotherapy if the level of expression of CD137L in the sample is higher than a reference level.

In some embodiments that may be combined with any of the preceding embodiments, the checkpoint blockade immunotherapy comprises administering an anti-PD-1 antibody or an anti-PD-L1 antibody. In some embodiments that may be combined with any of the preceding embodiments, the subject is further administered an effective amount of an anti-CD137 antibody.

In some embodiments that may be combined with any of the preceding embodiments, the subject is a human. In some embodiments that may be combined with any of the preceding embodiments, the sample is a serum sample. In some embodiments that may be combined with any of the preceding embodiments, the sample is a tumor sample. In some embodiments, the tumor sample is a tumor biopsy. In some embodiments that may be combined with any of the preceding embodiments, the sample comprises one or more cancer cells.

In some embodiments that may be combined with any of the preceding embodiments, the level of expression of CD137L is the level of protein expression of CD137L. In some embodiments, the level of protein expression is measured by a method selected from immunoassay, PET imaging, Western blotting, ELISA, immunohistochemistry, and flow cytometry. In some embodiments that may be combined with any of the preceding embodiments, the level of expression of CD137L is the level of RNA transcript expression of CD137L. In some embodiments, the level of transcript expression is measured by a method selected from RT-PCR, in situ hybridization, and next generation sequencing. In some embodiments that may be combined with any of the preceding embodiments, the level of expression of CD137L is the level of expression of CD137L by cancer cells (e.g., in a sample).

In another aspect, provided herein are anti-CD137 antibodies (e.g., according to any one of the above embodiments) for use in a method of treating or delaying progression of cancer in a subject in need thereof, the method comprising administering an effective amount of the anti-CD137 antibody to the subject if the level of expression of CD137 ligand (CD137L) in a sample obtained from the subject is lower than a reference level. Further provided herein are anti-CD137 antibodies (e.g., according to any one of the above embodiments) for use in a method of treating or delaying progression of cancer in a subject in need thereof, the method comprising administering an effective amount of the anti-CD137 antibody to the subject, wherein it has been determined that the subject is likely to respond to the anti-CD137 antibody when the level of expression of CD137L in a sample obtained from the subject is lower than a reference level.

In another aspect, provided herein are anti-CD137 antibodies (e.g., according to any one of the above embodiments) for use in the manufacture of a medicament for treating or delaying progression of cancer in a subject in need thereof, wherein the medicament is administered to the subject if the level of expression of CD137 ligand (CD137L) in a sample obtained from the subject is lower than a reference level. Further provided herein are anti-CD137 antibodies (e.g., according to any one of the above embodiments) for use in the manufacture of a medicament for treating or delaying progression of cancer in a subject in need thereof, wherein it has been determined that the subject is likely to respond to the anti-CD137 antibody when the level of expression of CD137L in a sample obtained from the subject is lower than a reference level.

It is to be understood that one, some, or all of the properties of the various embodiments described above and herein may be combined to form other embodiments of the present disclosure. These and other aspects of the present disclosure will become apparent to one of skill in the art. These and other embodiments of the present disclosure are further described by the detailed description that follows.

### BRIEF DESCRIPTION OF THE DRAWINGS

FIG. 1 shows CD137 ligand transcriptional expression status in tumor vs. normal tissues from clinical samples. The data were obtained from the Cancer Genome Atlas (TCGA). ACC, Adrenocortical carcinoma; BLCA, Bladder urothelial carcinoma; BRCA, Breast invasive carcinoma; CESC, Cervical and endocervical cancers; CHOL, Cholangiocarcinoma; COAD, Colon adenocarcinoma; COADREAD, Colorectal adenocarcinoma; DLBC, Lymphoid Neoplasm Diffused Large-B cell lymphoma; ESCA, Esophageal carcinoma; GBM, Glioblastoma multiforme; GBMLGG, Glioblastoma multiforme/low grade glioma; HNSC, Head and Neck squamous cell carcinoma; KICH, Kidney Chromophobe; KIPAN, Kidney Chromophobe/kidney renal clear cell carcinoma/kidney renal papillary cell carcinoma; KIRC, Kidney renal clear cell carcinoma; KIRP, Kidney renal papillary cell carcinoma; LAML, Acute myeloid leukemia; LGG, Low grade glioma; LIHC, Liver hepatocellular carcinoma; LUAD, Lung adenocarcinoma; LUSC, Lung squamous cell carcinoma; MESO, Mesothelioma; OV, Ovarian serous cystadenocarcinoma; PAAD, Pancreatic adenocarcinoma; PCPG, Pheochromocytoma and Paraganglioma; PRAD, Prostate adenocarcinoma; READ, Rectum adenocarcinoma; SARC, Sarcoma; SKCM, Skin Cutaneous Melanoma; STAD, Stomach adenocarcinoma; STES, Stomach and esophageal carcinoma; TGCT, Testicular Germ Cell Tumors; THCA, Thyroid carcinoma; THYM, Thymoma; UCEC, Uterine Corpus Endometrial Carcinoma; UCS, Uterine carcinosarcoma; and UVM, Uveal Melanoma.

FIGS. 2A-D show CD137 ligand-mediated cell signaling in co-cultures of human B-cell lymphoma (Daudi or Raji) cells and 293T cells with a stably-integrated NFκB-dependent luciferase reporter that were transiently transfected with a plasmid expressing human CD137. FIG. 2A shows luciferase activity in co-cultures of Daudi and 293T reporter cells incubated at various cell ratios (0:1, 1:1, 3:1, and 10:1 Daudi:293T cells), and treated with the indicated concentrations of a human IgG4 isotype control antibody. FIG. 2B shows luciferase activity in co-cultures of Daudi and 293T reporter cells incubated at various cell ratios (0:1, 1:1, 3:1, and 10:1 Daudi:293T cells), and treated with the indicated concentrations of a blocking anti-CD137 antibody. FIG. 2C shows luciferase activity in co-cultures of Raji and 293T reporter cells incubated at various cell ratios (0:1, 1:1, 3:1, and 10:1 Raji:293T cells), and treated with the indicated concentrations of a human IgG4 isotype control antibody. FIG. 2D shows luciferase activity in co-cultures of Raji and 293T reporter cells incubated at various cell ratios (0:1, 1:1, 3:1, and 10:1 Raji:293T cells), and treated with the indicated concentrations of a blocking anti-CD137 antibody. NFκB-dependent luciferase activity was measured, after overnight incubation in the co-cultured cells.

FIG. 3 shows expression profiles of CD137L on various mouse tumor cell lines, as measured by flow cytometry. The grey line indicates control staining; the black line indicates anti-mouse CD137L staining.

FIGS. 4A-4B show the in vivo anti-tumor activity of an anti-CD137 agonist antibody (αCD137-AB1), or isotype control antibody, in various syngenic mouse tumor models. FIG. 4A shows the syngenic mouse tumor models that were categorized as non-responders to the anti-CD137 agonist antibody treatment (models showing a tumor growth inhibition (TGI)<20% after administration of αCD137-AB1 vs. isotype control antibody). FIG. 4B shows the syngenic mouse tumor models that were categorized as responders to the anti-CD137 agonist antibody treatment (models showing a TGI>35% after administration of αCD137-AB1 vs. isotype control antibody).

FIGS. 5A-5B show the in vivo anti-tumor activity of checkpoint blockade immunotherapies, or vehicle controls (PBS), in the syngenic Pan02 mouse pancreatic tumor model. FIG. 5A shows the in vivo activity of a surrogate anti-mouse PD-L1 antibody (clone 10F.9G2) in the syngenic Pan02 mouse pancreatic tumor model. FIG. 5B shows the in vivo activity of a surrogate anti-mouse PD-1 antibody (clone RMP1-14) in the syngenic Pan02 mouse pancreatic tumor model.

FIGS. 6A-6D show the in vivo anti-tumor activity of an anti-CD137 agonist antibody (αCD137-AB1), isotype control antibody, or combination of anti-CD137 agonist antibody (αCD137-AB1) and other agents in various syngeneic mouse tumor models. FIG. 6A shows the combination study of anti-CD137 agonist antibody (αCD137-AB1) with anti-PD-L1 (left) and anti-CD20 (right) in LL/2 mouse lung cancer tumor model. FIG. 6B shows the combination study of anti-CD137 agonist antibody (αCD137-AB1) with anti-PD1 (clone RMP1-14) in PD1-resistant CT26 mouse colon cancer tumor model (Charles River). FIGS. 6C & 6D show the combination study of anti-CD137 agonist antibody (αCD137-AB1) with anti-CTLA4 (FIG. 6C) or radiation (FIG. 6D) in MC38 mouse colon cancer tumor model.

FIG. 7 shows representative immunohistochemical (IHC) staining images of CD137L from normal human tissues and tumors. FFPE sections from human normal breast, lung, ovary and endometrium tissues, as well as respective tumors from different patients were processed and stained with mouse anti-human CD137L antibody, followed by detection with HRP-labeled anti-mouse secondary antibody and subsequent DAB chromogen. The tissue slides were counterstained with Hematoxylin.

FIG. 8 shows the results of testing αCD137-AB1 in competition assays in which anti-CD137 antibodies compete in a concentration dependent manner with the binding interaction between CD137 ligand and its receptor using a stable transfected cell line.

### DETAILED DESCRIPTION

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

### II. Definitions

Before describing the present disclosure in detail, it is to be understood that this present disclosure is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure described herein include “comprising,” “consisting,” and “consisting essentially of” aspects and embodiments.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “antibody” is used herein in the broadest sense and specifically covers monoclonal antibodies (including full length monoclonal antibodies), polyclonal antibodies, multispecific antibodies (e.g., bispecific antibodies), and antibody fragments (e.g., a single-chain variable fragment or scFv) so long as they exhibit the desired biological activity.

The term “antibody” is an art-recognized term and may refer to an antigen-binding protein (i.e, immunoglobulin) having a basic four-polypeptide chain structure consisting of two identical heavy (H) chains and two identical light (L) chains. Each L chain is linked to an H chain by one covalent disulfide bond, while the two H chains are linked to each other by one or more disulfide bonds depending on the H chain isotype. Each heavy chain has, at the N-terminus, a variable region (abbreviated herein as V_{H}) followed by a constant region. The heavy chain constant region is comprised of three domains, C_{H1}, C_{H2} and C_{H3}. Each light chain has, at the N-terminus, a variable region (abbreviated herein as V_{L}) followed by a constant region at its other end. The light chain constant region is comprised of one domain, C_{L}. The V_{L} is aligned with the V_{H} and the C_{L} is aligned with the first constant domain of the heavy chain (CH1). The pairing of a V_{H} and V_{L} together forms a single antigen-binding site. An IgM antibody consists of 5 of the basic heterotetramer units along with an additional polypeptide called J chain, and therefore contains 10 antigen binding sites, while secreted IgA antibodies can polymerize to form polyvalent assemblages comprising 2-5 of the basic 4-chain units along with J chain.

The V_{H} and V_{L} regions can be further subdivided into regions of hypervariability, termed hyper-variable regions (HVR) based on the structural and sequence analysis. HVRs are interspersed with regions that are more conserved, termed framework regions (FW). For comparison, the Kabat CDR definition by Yvonne Chen, et al. (Selection and Analysis of an Optimized Anti-VEGF Antibody: Crystal Structure of an Affinity-matured Fab in Complex with Antigen, J. Mol. Biol. (1999) 293, 865-881) is listed below. Each V_{H} and V_{L}, is composed of three HVRs and four FWs, arranged from amino-terminus to carboxy-terminus in the following order: FW1, HVR1, FW2, HVR2, FW3, HVR3, FW4. Throughout the present disclosure, the three HVRs of the heavy chain are referred to as HVR_H1, HVR_H2, and HVR_H3. Similarly, the three HVRs of the light chain are referred to as HVR_L1, HVR_L2, and HVR_L3.

The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. Within light and heavy chains, the variable and constant regions are joined by a “J” region of about 12 or more amino acids, with the heavy chain also including a “D” region of about 10 or more amino acids. See generally, Fundamental Immunology Ch. 7 (Paul, W., ed., 2^{nd} ed. Raven Press, N.Y. (1989)).

The L chain from any vertebrate species can be assigned to one of two clearly distinct types, called kappa and lambda, based on the amino acid sequences of their constant domains. Depending on the amino acid sequence of the constant domain of their heavy chains (CH), antibodies can be assigned to different classes or isotypes. There are five classes of antibodies: IgA, IgD, IgE, IgG, and IgM, having heavy chains designated α (alpha), δ (delta), ε (epsilon), γ (gamma), and μ (mu), respectively. The IgG class of antibody can be further classified into four subclasses IgG1, IgG2, IgG3, and IgG4 by the gamma heavy chains, Y1-Y4, respectively.

The term “antibody derivative” or “derivative” of an antibody refers to a molecule that is capable of binding to the same antigen (e.g., CD137) that the antibody binds to and comprises an amino acid sequence of the antibody linked to an additional molecular entity. The amino acid sequence of the antibody that is contained in the antibody derivative may be a full-length heavy chain, a full-length light chain, any portion or portions of a full-length heavy chain, any portion or portions of the full-length light chain of the antibody, any other fragment(s) of an antibody, or the complete antibody. The additional molecular entity may be a chemical or biological molecule. Examples of additional molecular entities include chemical groups, amino acids, peptides, proteins (such as enzymes, antibodies), and chemical compounds. The additional molecular entity may have any utility, such as for use as a detection agent, label, marker, pharmaceutical or therapeutic agent. The amino acid sequence of an antibody may be attached or linked to the additional molecular entity by chemical coupling, genetic fusion, noncovalent association, or otherwise. The term “antibody derivative” also encompasses chimeric antibodies, humanized antibodies, and molecules that are derived from modifications of the amino acid sequences of an antibody (e.g., a CD137 antibody), such as conservation amino acid substitutions, additions, and insertions.

The term “antigen-binding fragment” or “antigen binding portion” of an antibody refers to one or more portions of an antibody that retain the ability to bind to the antigen that the antibody bonds to (e.g., CD137). Examples of “antigen-binding fragment” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., Nature 341:544-546 (1989)), which consists of a V_{H} domain; and (vi) an isolated complementarity determining region (CDR).

The term “binding molecule” encompasses (1) antibody, (2) antigen-binding fragment of an antibody, and (3) derivative of an antibody, each as defined herein.

The term “binding CD137,” “binds CD137,” “binding to CD137,” or “binds to CD137” refers to the binding of a binding molecule, as defined herein, to the human CD137 in an in vitro assay, such as a Biacore assay, with an affinity (K_{D}) of 100 nM or less.

The terms “CD137” and “CD137 receptor” are used interchangeably in the present application, and include the human CD137 receptor, as well as variants, isoforms, and species homologs thereof. Accordingly, a binding molecule, as defined and disclosed herein, may also bind CD137 from species other than human. In other cases, a binding molecule may be completely specific for the human CD137 and may not exhibit species or other types of cross-reactivity.

The term “CD137 antibody” refers to an antibody, as defined herein, capable of binding to human CD137 receptor.

The term “chimeric antibody” refers to an antibody that comprises amino acid sequences derived from different animal species, such as those having a variable region derived from a human antibody and a murine immunoglobulin constant region.

The term “compete for binding” refers to the interaction of two antibodies in their binding to a binding target. A first antibody competes for binding with a second antibody if binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not, be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope, whether to the same, greater, or lesser extent, the antibodies are said to “cross-compete” with each other for binding of their respective epitope(s).

The term “epitope” refers to a part of an antigen to which an antibody (or antigen-binding fragment thereof) binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope can include various numbers of amino acids in a unique spatial conformation. Methods of determining spatial conformation of epitopes include, for example, x-ray crystallography, 2-dimensional nuclear magnetic resonance, deuterium and hydrogen exchange in combination with mass spectrometry, or site-directed mutagenesis, or all methods used in combination with computational modeling of antigen and its complex structure with its binding antibody and its variants. See, e.g., Epitope Mapping Protocols in Methods in Molecular Biology, Vol. 66, G. E. Morris, Ed. (1996). Once a desired epitope of an antigen is determined, antibodies to that epitope can be generated, e.g., using the techniques described herein. The generation and characterization of antibodies may also elucidate information about desirable epitopes. From this information, it is then possible to competitively screen antibodies for binding to the same epitope. An approach to achieve this is to conduct cross-competition studies to find antibodies that competitively bind with one another, i.e., the antibodies compete for binding to the antigen. A high throughput process for “binning” antibodies based upon their cross-competition is described in PCT Publication No. WO 03/48731.

The term “germline” refers to the nucleotide sequences of the antibody genes and gene segments as they are passed from parents to offspring via the germ cells. The germline sequence is distinguished from the nucleotide sequences encoding antibodies in mature B cells which have been altered by recombination and hypermutation events during the course of B cell maturation.

The term “glycosylation sites” refers to amino acid residues which are recognized by a eukaryotic cell as locations for the attachment of sugar residues. The amino acids where carbohydrate, such as oligosaccharide, is attached are typically asparagine (N-linkage), serine (O-linkage), and threonine (O-linkage) residues. The specific site of attachment is typically signaled by a sequence of amino acids, referred to herein as a “glycosylation site sequence”. The glycosylation site sequence for N-linked glycosylation is: -Asn-X-Ser- or -Asn-X-Thr-, where X may be any of the conventional amino acids, other than proline. The terms “N-linked” and “O-linked” refer to the chemical group that serves as the attachment site between the sugar molecule and the amino acid residue. N-linked sugars are attached through an amino group; O-linked sugars are attached through a hydroxyl group. The term “glycan occupancy” refers to the existence of a carbohydrate moiety linked to a glycosylation site (i.e., the glycan site is occupied). Where there are at least two potential glycosylation sites on a polypeptide, either none (0-glycan site occupancy), one (1-glycan site occupancy) or both (2-glycan site occupancy) sites can be occupied by a carbohydrate moiety.

The term “host cell” refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell.”

The term “human antibody” refers to an antibody in which the entire amino acid sequences of the light chains and heavy chains are from the human immunoglobulin genes. A human antibody may contain murine carbohydrate chains if produced in a mouse, in a mouse cell or in a hybridoma derived from a mouse cell. Human antibodies may be prepared in a variety of ways known in the art.

The term “humanized antibody” refers to a chimeric antibody that contains amino acid residues derived from human antibody sequences. A humanized antibody may contain some or all of the CDRs or HVRs from a non-human animal or synthetic antibody while the framework and constant regions of the antibody contain amino acid residues derived from human antibody sequences.

The term “illustrative antibody” refers to any one of the antibodies described in the disclosure. These antibodies may be in any class (e.g., IgA, IgD, IgE, IgG, and IgM). Thus, each antibody identified above encompasses antibodies in all five classes that have the same amino acid sequences for the V_{L}, and V_{H} regions. Further, the antibodies in the IgG class may be in any subclass (e.g., IgG1 IgG2, IgG3, and IgG4). Thus, each antibody identified above in the IgG subclass encompasses antibodies in all four subclasses that have the same amino acid sequences for the V_{L}, and V_{H} regions. The amino acid sequences of the heavy chain constant regions of human antibodies in the five classes, as well as in the four IgG subclasses, are known in the art.

The term “isolated antibody” or “isolated binding molecule” refers to an antibody or a binding molecule, as defined herein, that: (1) is not associated with naturally associated components that accompany it in its native state; (2) is free of other proteins from the same species; (3) is expressed by a cell from a different species; or (4) does not occur in nature. Examples of isolated antibodies include a CD137 antibody that has been affinity purified using CD137, a CD137 antibody that has been generated by hybridomas or other cell line in vitro, and a CD137 antibody derived from a transgenic animal.

The term “kₐ” refers to the association rate constant of a particular antibody-antigen interaction, whereas the term “k_{d}” refers to the dissociation rate constant of a particular antibody-antigen interaction.

The term “K_{D}” refers to the equilibrium dissociation constant of a particular antibody-antigen interaction. It is obtained from the ratio of k_{d} to kₐ (i.e., k_{d}/kₐ) and is expressed as a molar concentration (M). K_{D} is used as a measure for the affinity of an antibody's binding to its binding partner. The smaller the K_{D}, the more tightly bound the antibody is, or the higher the affinity between antibody and the antigen. For example, an antibody with a nanomolar (nM) dissociation constant binds more tightly to a particular antigen than an antibody with a micromolar (μM) dissociation constant. K_{D} values for antibodies can be determined using methods well established in the art. One method for determining the K_{D} of an antibody is by using surface plasmon resonance, typically using a biosensor system such as a Biacore® system.

The term “prevent” or “preventing,” with reference to a certain disease condition in a mammal, refers to preventing or delaying the onset of the disease, or preventing the manifestation of clinical or subclinical symptoms thereof.

As used herein, “sequence identity” between two polypeptide sequences indicates the percentage of amino acids that are identical between the sequences. The amino acid sequence identity of polypeptides can be determined conventionally using known computer programs such as Bestfit, FAST A, or BLAST (see, e.g. Pearson, Methods' Enzymol. 183:63-98 (1990); Pearson, Methods Mol. Biol. 132:185-219 (2000); Altschul et al., J. Mol. Biol. 215:403-410 (1990); Altschul et al., Nucleic Acids Res. 25:3389-3402 (1997)). When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, fragments, or variants thereof disclosed herein.

The term “specifically binds” or “specifically binds to,” in reference to the interaction of a binding molecule, as defined herein, (e.g., an antibody) with its binding partner (e.g., an antigen), refers to the ability of the binding molecule to discriminate between an antigen of interest from an animal species and the antigen orthologue from a different animal species under a given set of conditions. A CD137 binding molecule is said to specifically bind to human CD137 if it binds to human CD137 at an EC50 that is below 50 percent of the EC50 at which it binds CD137 of rat or mouse as determined in an in vitro assay. Binding specificity of an antibody can be determined using methods known in the art. Examples of such methods include FACS using PHA stimulated primary cells, Western blots, ELISA-, RIA-, ECL-, IRMA-tests and peptide scans.

The term “selectively binds” or “selectively binds to,” in reference to the interaction of a binding molecule, as defined herein, (e.g., an antibody) with its binding partner (e.g., an antigen), refers to the ability of the binding molecule to discriminate between an antigen of interest from an animal species (such as human CD137) and a different antigen from the same animal species (such as human CD40) under a given set of conditions. A CD137 binding molecule is said to selectively bind to human CD137 if it binds to human CD137 at an EC50 that is below 10 percent of the EC50 at which it binds to human CD40 or human CD134 as determined in an in vitro assay.

The term “treat”, “treating”, or “treatment”, with reference to a certain disease condition in a mammal, refers causing a desirable or beneficial effect in the mammal having the disease condition. The desirable or beneficial effect may include reduced frequency or severity of one or more symptoms of the disease (i.e., tumor growth and/or metastasis, or other effect mediated by the numbers and/or activity of immune cells, and the like), or arrest or inhibition of further development of the disease, condition, or disorder. In the context of treating cancer in a mammal, the desirable or beneficial effect may include inhibition of further growth or spread of cancer cells, death of cancer cells, inhibition of reoccurrence of cancer, reduction of pain associated with the cancer, or improved survival of the mammal. The effect can be either subjective or objective. For example, if the mammal is human, the human may note improved vigor or vitality or decreased pain as subjective symptoms of improvement or response to therapy. Alternatively, the clinician may notice a decrease in tumor size or tumor burden based on physical exam, laboratory parameters, tumor markers or radiographic findings. Some laboratory signs that the clinician may observe for response to treatment include normalization of tests, such as white blood cell count, red blood cell count, platelet count, erythrocyte sedimentation rate, and various enzyme levels. Additionally, the clinician may observe a decrease in a detectable tumor marker. Alternatively, other tests can be used to evaluate objective improvement, such as sonograms, nuclear magnetic resonance testing and positron emissions testing.

The term “vector” refers to a nucleic acid molecule capable of transporting a foreign nucleic acid molecule. The foreign nucleic acid molecule is linked to the vector nucleic acid molecule by a recombinant technique, such as ligation or recombination. This allows the foreign nucleic acid molecule to be multiplied, selected, further manipulated or expressed in a host cell or organism. A vector can be a plasmid, phage, transposon, cosmid, chromosome, virus, or virion. One type of vectors can be integrated into the genome of a host cell upon introduction into the host cell, and thereby are replicated along with the host genome (e.g., non-episomal mammalian vectors). Another type of vector is capable of autonomous replication in a host cell into which it is introduced (e.g., bacterial vectors having a bacterial origin of replication and episomal mammalian vectors). Another specific type of vector capable of directing the expression of expressible foreign nucleic acids to which they are operatively linked is commonly referred to as “expression vectors.” Expression vectors generally have control sequences that drive expression of the expressible foreign nucleic acids. Simpler vectors, known as “transcription vectors,” are only capable of being transcribed but not translated: they can be replicated in a target cell but not expressed. The term “vector” encompasses all types of vectors regardless of their function. Vectors capable of directing the expression of expressible nucleic acids to which they are operatively linked are commonly referred to “expression vectors.”

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. The term “amino acid analogs” refers to compounds that have the same basic chemical structure as a naturally occurring amino acid but the C-terminal carboxy group, the N-terminal amino group, or side chain functional group has been chemically modified to another functional group. The term “amino acid mimetics” refers to chemical compounds that have a structure that is different from the general chemical structure of an amino acid, but that functions similarly to a naturally occurring amino acid.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)).

The terms “polypeptide,” “protein,” and “peptide” are used interchangeably herein and may refer to polymers of two or more amino acids.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “isolated nucleic acid” refers to a nucleic acid molecule of genomic, cDNA, or synthetic origin, or a combination thereof, which is separated from other nucleic acid molecules present in the natural source of the nucleic acid. For example, with regard to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid of interest.

As used herein, the term “biomarker” or “marker” refers generally to a molecule (e.g., pre-mRNA, mRNA, protein, etc.), the expression of which in or on a subject's tissue or cell, or secreted by the subject's tissue or cell, can be detected by known methods (or methods disclosed herein) and is predictive or can be used to predict (or aid prediction) for a subject's sensitivity to, and in some embodiments, to predict (or aid prediction) a subject's responsiveness to, treatment regimens (e.g., treatments comprising anti-CD137 antibodies, treatments comprising checkpoint blockade immunotherapy, etc.).

As used herein, the term “sample”, refers to a composition that is obtained or derived from a subject of interest that contains a cellular and/or other molecular entity that is to be characterized and/or identified, for example based on physical, biochemical, chemical and/or physiological characteristics.

As used herein, the term “tissue or cell sample” refers to a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells. Optionally, the tissue or cell sample is obtained from a disease tissue or organ. The tissue sample may contain compounds which are not naturally intermixed with the tissue in nature such as preservatives, anticoagulants, buffers, fixatives, nutrients, antibiotics, or the like.

As used herein, a “subject”, “patient”, or “individual” may refer to a human or a non-human animal. A “non-human animal” may refer to any animal not classified as a human, such as domestic, farm, or zoo animals, sports, pet animals (such as dogs, horses, cats, cows, etc.), as well as animals used in research. Research animals may refer without limitation to nematodes, arthropods, vertebrates, mammals, frogs, rodents (e.g., mice or rats), fish (e.g., zebrafish or pufferfish), birds (e.g., chickens), dogs, cats, and non-human primates (e.g., rhesus monkeys, cynomolgus monkeys, chimpanzees, etc.). In some embodiments, the subject, patient, or individual is a human.

The term “mammal” refers to any animal species of the Mammalia class. Examples of mammals include: humans; laboratory animals such as rats, mice, simians and guinea pigs; domestic animals such as cats, dogs, rabbits, cattle, sheep, goats, horses, and pigs; and captive wild animals such as lions, tigers, elephants, and the like.

As used herein, a “reference value” or “reference level” may be an absolute value; a relative value; a value that has an upper and/or lower limit; a range of values; an average value; a median value; a mean value; or a value as compared to a particular level or baseline level.

An “effective amount” refers to at least an amount effective, at dosages and for periods of time necessary, to achieve a one or more desired or indicated effect, including a therapeutic or prophylactic result. An effective amount can be provided in one or more administrations. For purposes of the present disclosure, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

### III. Methods

Overview

Certain aspects of the present disclosure relate to methods of treating or delaying progression of cancer in a subject by administering an effective amount of an anti-cancer therapy (e.g., anti-CD137 antibody therapy, checkpoint blockade immunotherapy) based on the level of expression of CD137L in one or more samples obtained from the subject.

Other aspects of the present disclosure relate to methods of determining whether a subject is likely to respond to certain anti-cancer therapies (e.g., anti-CD137 antibody therapy, checkpoint blockade immunotherapy) by measuring CD137L expression in one or more samples obtained from the subject.

Other aspects of the present disclosure relate to methods of treating or delaying progression of cancer in a subject by administering an effective amount of an anti-cancer therapy (e.g., anti-CD137 antibody therapy, checkpoint blockade immunotherapy) after it has been determined that the subject is likely to respond to the therapy.

Other aspects of the present disclosure relate to selecting a subject to receive or not receive certain anti-cancer therapies (e.g., anti-CD137 antibody therapy, checkpoint blockade immunotherapy) based on the level of expression of CD137L in one or more samples obtained from the subject.

Other aspects of the present disclosure relate to methods for predicting responsiveness and/or monitoring treatment and/or responsiveness of a subject to certain anti-cancer therapies (e.g., anti-CD137 antibody therapy, checkpoint blockade immunotherapy) by measuring CD137L expression in one or more samples obtained from the subject.

Yet other aspects of the present disclosure relate to methods of positively and/or negatively stratifying patients into particular treatment regimen groups based upon the level of CD137L expression in one or more samples obtained from the patients.

Measuring CD137L Expression

In some embodiments, the present disclosure relates to measuring the level of CD137L expression in a sample. In some embodiments, measuring the level of expression of CD137L in a sample comprises measuring the level of expression of a nucleic acid molecule encoding CD137L (e.g., measuring the level of RNA (such as pre-mRNA or mRNA) transcript expression from a gene encoding CD137L) and/or measuring the level of protein expression of CD137L. The nucleic acid sequence of an exemplary nucleic acid encoding human CD137L, as well as the amino acid sequence of an exemplary human CD137L polypeptide, are shown below:

CD137L nucleic acid sequence:
(SEQ ID NO: 529)
Atggaatacgcctctgacgcttcactggaccccgaagccccgtggcctcc
cgcgccccgcgctcgcgcctgccgcgtactgccttgggccctggtcgcgg
ggctgctgctgctgctgctgctcgctgccgcctgcgccgtcttcctcgcc
tgcccctgggccgtgtccggggctcgcgcctcgcccggctccgcggccag
cccgagactccgcgagggtcccgagcMcgcccgacgatcccgccggcctc
ttggacctgcggcagggcatgtttgcgcagctggtggcccaaaatgttct
gctgatcgatgggcccctgagctggtacagtgacccaggcctggcaggcg
tgtccctgacggggggcctgagctacaaagaggacacgaaggagctggtg
gtggccaaggctggagtctactatgtcttctttcaactagagctgcggcg
cgtggtggccggcgagggctcaggctccgtttcacttgcgctgcacctgc
agccactgcgctctgctgctggggccgccgccctggctttgaccgtggac
ctgccacccgcctcctccgaggctcggaactcggccttcggtttccaggg
ccgcttgctgcacctgagtgccggccagcgcctgggcgtccatcttcaca
ctgaggccagggcacgccatgcctggcagcttacccagggcgccacagtc
ttgggactcttccgggtgacccccgaaatcccagccggactcccttcacc
gaggtcggaa
CD137L polypeptide sequence:
(SEQ ID NO: 530)
MEYASDASLDPEAPWPPAPRARACRVLPWALVAGLLLLLLLAAACAVFLA
CPWAVSGARASPGSAASPRLREGPELSPDDPAGLLDLRQGMFAQLVAQNV
LL1DGPLSWYSDPGLAGVSLTGGLSYKEDTKELVVAKAGVYYVFFQLELR
RVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPS
PRSE

In some embodiments, the level of expression of CD137L in a sample is measured by determining the level of RNA transcript expression of CD137L. Suitable methods of measuring RNA transcript levels in a sample are known in the art, including, for example, by Northern blot analysis, nuclease protection assays, in situ hybridization, PCR analysis (e.g., qPCR, RT-PCR, RT-qPCR, etc.), and next generation sequencing (e.g., RNAseq). In some embodiments, the level of transcript expression of CD137L is measured by RT-PCR, in situ hybridization, and/or RNAseq.

In some embodiments, the level of expression of CD137L in a sample is measured by determining the level of protein expression of CD137L. Suitable methods of measuring protein expression in a sample are known in the art, including, for example, immunoassays, immunohistochemistry (IHC), PET imaging, Western blotting, enzyme-linked immunosorbent assays (ELISAs), flow cytometry, and mass spectrometry. In some embodiments, the level of CD137L protein expression is measured by immunoassay, Western blotting, ELISA, IHC, and/or flow cytometry.

Subjects

In some embodiments, the present disclosure relates to subjects suffering from or believed to be suffering from cancer. In some embodiments, the subject has been diagnosed with cancer. In some embodiments, the subject has not been diagnosed with cancer. In some embodiments, the subject is at risk of developing cancer.

The subject may be suffering from, or believed to be suffering from, any cancer known in the art, including, for example, lung cancers such as bronchogenic carcinoma (e.g., squamous cell carcinoma, small cell carcinoma, large cell carcinoma, and adenocarcinoma), alveolar cell carcinoma, bronchial adenoma, chondromatous hamartoma (noncancerous), mesothelioma, and sarcoma (cancerous); heart cancer such as myxoma, fibromas, and rhabdomyomas; bone cancers such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, chordomas, germinomas, teratomas, dermoid cysts, and angiomas; adrenal cancers (e.g., adrenocortical carcinoma); cancers in digestive system such as esophageal carcinoma, leiomyoma, epidermoid carcinoma, adenocarcinoma, leiomyosarcoma, stomach adenocarcinomas, intestinal lipomas, intestinal neurofibromas, intestinal fibromas, polyps in large intestine, colon and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal papillary cell carcinoma, renal cell carcinoma, renal clear cell carcinoma, hypernephroma, and transitional cell carcinoma of the renal pelvis; bladder cancers; hematological cancers such as acute lymphocytic (lymphoblastic) leukemia, acute myeloid (myelocytic, myelogenous, myeloblastic, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, B cell lymphoma, Diffused Large B cell lymphoma, T cell lymphoma, mycosis fungoides, and myeloproliferative disorders (including myeloproliferative disorders such as polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (e.g., seminoma, teratoma, embryonal carcinoma, germ cell tumors, and choriocarcinoma); breast cancer; female reproductive system cancers such as uterine cancer (endometrial carcinoma, uterine carcinosarcoma), cervical cancer (cervical carcinoma), cancer of the ovaries (ovarian carcinoma, ovarian serous cystadenocarcinoma), vulvar carcinoma, vaginal carcinoma, fallopian tube cancer, and hydatidiform mole; thyroid cancer (including papillary, follicular, anaplastic, or medullary cancer) and thymoma; pheochromocytomas (adrenal gland) and paraganglioma; noncancerous growths of the parathyroid glands; and pancreatic cancers.

In some embodiments, the subject has not previously received one or more anti-cancer therapies. In some embodiments, the subject has previously received and/or is currently receiving one or more anti-cancer therapies.

In some embodiments, the methods of the present disclosure are useful for: identifying subjects with cancers that are likely or unlikely to respond to treatment with an anti-CD137 antibody therapy (e.g., as described herein) and/or a checkpoint blockade immunotherapy (e.g., as described herein); aiding in subject selection for administering an anti-CD137 antibody therapy and/or a checkpoint blockade immunotherapy; predicting the likelihood of success when treating a subject with an anti-CD137 antibody therapy and/or a checkpoint blockade immunotherapy; assessing or monitor disease progression in a subject treated with an anti-CD137 antibody therapy and/or a checkpoint blockade immunotherapy; assessing or monitor treatment efficacy using an anti-CD137 antibody therapy and/or a checkpoint blockade immunotherapy; and/or determining prognosis of a subject.

Samples Obtained from a Subject

In some embodiments, the present disclosure relates to measuring the level of CD137L expression in a sample obtained from a subject. In some embodiments, the level of expression of CD137L is measured in one or more (e.g., one or more, two or more, three or more, four or more, etc.) samples obtained from a subject. Any suitable sample in the form of tissues and/or fluids that are known or believed to contain diseased cells and/or the target of interest (e.g., full length CD137L, CD137L fragments including soluble CD137L fragments) may be used in the methods described herein, including, for example, sputum, pleural fluid, lymph fluid, bone marrow, blood, plasma, serum, urine, tissue samples (samples known or expected to contain cancer cells), tumor samples, tumor biopsies, etc. In some embodiments, the sample is a serum sample. In some embodiments, the sample is a tumor sample. In some embodiments, the sample is a tumor biopsy. In some embodiments, the sample comprises one or more cancer cells.

Methods of obtaining suitable tissue and/or fluid samples (e.g., methods that are appropriate for obtaining a representative sample from a particular type, location, disease tissue, etc.) are well known to one of ordinary skill in the art, including, for example, by resection, bone marrow biopsy or bone marrow aspiration, endoscopic biopsy or endoscopic aspiration (e.g., cystoscopy, bronchoscopy, colonoscopy, etc.), needle biopsy or needle aspiration (e.g., fine needle aspiration, core needle biopsy, vacuum-assisted biopsy, image-guided biopsy, etc.) skin biopsy (e.g., shave biopsy, punch biopsy, incisional biopsy, excisional biopsy, etc.), various other surgical tissue (e.g., tumor tissue) biopsy and/or excision strategies, and fluid collections (e.g., collecting urine, blood, serum, plasma, sputum, etc.).

In some embodiments, the one or more samples obtained from the subject are enriched for diseased (e.g., cancerous) cells. Methods of enriching a tissue or fluid preparation for diseased (e.g., cancerous) cells are known in the art, including, for example, by separating diseased (e.g., cancerous) cells from normal cells by flow cytometry. In some embodiments, the level of expression of CD137L is measured in the enriched samples. In some embodiments, the level of expression of CD137L is measured in samples that have not been enriched or otherwise altered after isolation.

In some embodiments, the one or more samples are fixed (i.e. preserved) by conventional methodology (See e.g., “Manual of Histological Staining Method of the Armed Forces Institute of Pathology,” 3^{rd} edition (1960) Lee G. Luna, HT (ASCP) Editor, The Blakston Division McGraw-Hill Book Company, New York; The Armed Forces Institute of Pathology Advanced Laboratory Methods in Histology and Pathology (1994) Ulreka V. Mikel, Editor, Armed Forces Institute of Pathology, American Registry of Pathology, Washington, D.C.). The choice of a fixative may be determined by one of ordinary skill in the art for the purpose for which the sample is to be analyzed. The length of fixation will depend upon the size and type of the tissue sample and the fixative used (e.g., neutral buffered formalin, paraformaldehyde, etc.), as will be appreciated by one of ordinary skill in the art. In some embodiments, the level of expression of CD137L is measured in a sample that is fixed. In some embodiments, the level of expression of CD137L is measured in samples that have not been fixed or otherwise altered after isolation.

In some embodiments, one or more samples are obtained from the subject prior to administration with an anti-cancer therapy (e.g., an anti-CD137 antibody therapy and/or a checkpoint blockade immunotherapy). In some embodiments, one or more samples are obtained from the subject after administration of a first and/or subsequent does of an anti-cancer therapy (e.g., an anti-CD137 antibody therapy and/or a checkpoint blockade immunotherapy). In some embodiments, one or more samples are obtained from the subject after completion of an anti-cancer therapy regimen (e.g., an anti-CD137 antibody therapy and/or a checkpoint blockade immunotherapy). In some embodiments, one or more samples are obtained from the subject, prior to, during, and after completion of an anti-cancer therapy regimen (e.g., an anti-CD137 antibody therapy and/or a checkpoint blockade immunotherapy).

Comparison to a Reference Level

In some embodiments, the present disclosure relates to comparing the level of expression of CD137L in a sample obtained from a subject to a reference level of expression of CD137L. In some embodiments, the reference level is the level of expression of CD137L in a reference sample (e.g., a reference cell (such as a cell line, including but not limited to Raji (ATCC, CC-86) or Daudi (ATCC, CCL-213) cell lines), a corresponding sample taken from one or more patients determined to be responsive to anti-CD137 antibody therapy, a corresponding sample taken from one or more patients determined to be non-responsive to anti-CD137 antibody therapy, a corresponding adjacent normal tissue, etc.). In some embodiments, the reference level is measured in the reference sample using the same method as was used to measure the level of expression of CD137L in the subject's sample. In some embodiments, the reference level is measured in the reference sample using a different method than was used to measure the level of expression of CD137L in the subject's sample.

Without wishing to be bound to theory, it is thought that anti-CD137 antibody treatment may not lead to downregulation of CD137 on immune cells as significantly as the CD137L:CD137 interaction. As such, a tumor or cancer cell that has low expression of CD137L may interact with anti-tumor T cells that have higher CD137 (e.g., as compared to a tumor or cancer cell with higher levels of CD137L expression that cause downregulation of CD137 in interacting cells). In addition, the use of an anti-CD137 agonist antibody that blocks CD137L binding to CD137 may be advantageous, in that it stimulates CD137 signaling while blocking CD137L-mediated downregulation of CD137 on responding cells.

In some embodiments, the reference level is the level of expression of CD137L (e.g., average level of expression) on one or more reference cells. In some embodiments, the one or more reference cells are cells taken from a diseased tissue isolated from a cancer patient (e.g., one or more cancer cells from a patient suffering from adrenocortical carcinoma, bladder urothelial carcinoma, breast invasive carcinoma, cervical or endocervical cancers, cholangiocarcinoma, colon adenocarcinoma, colorectal adenocarcinoma, lymphoid neoplasm diffused large-B cell lymphoma, esophageal carcinoma, glioblastoma multiforme and/or low grade glioma, head and Neck squamous cell carcinoma, kidney chromophobe, kidney renal clear cell carcinoma, kidney renal papillary cell carcinoma, acute myeloid leukemia, low grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma or paraganglioma, prostate adenocarcinoma, rectum adenocarcinoma, sarcoma, skin cutaneous melanoma, stomach adenocarcinoma, stomach or esophageal carcinoma, testicular germ cell tumors, thyroid carcinoma, thymoma, uterine corpus endometrial carcinoma, uterine carcinosarcoma, uveal melanoma, etc.). In some embodiments, the one or more reference cells are one or more cells from a cancer cell line (e.g., a liver cancer cell line, a colon cancer cell line, a melanoma cell line, a lung cancer cell line, a pancreatic cancer cell line, a prostate cancer cell line, a B cell lymphoma cell line, a T cell lymphoma cell line, etc.). In some embodiments, the one or more reference cells are one or more cells of an adjacent normal tissue in the subject (e.g., comparing CD137L expression (such as by immunohistochemical staining) in a tumor sample from the patient to CD137L expression in a normal tissue adjacent to the tumor, etc.).

In some embodiments, the reference level is the level of expression of CD137L in one or more samples isolated from one or more patients determined to be responsive to anti-CD137 antibody therapy (e.g., one or more samples isolated from one or more patients determined by a clinician to be responsive to anti-CD137 antibody therapy (such as patients receiving treatment with an anti-CD137 antibody in a clinical trial)). In some embodiments, the reference level is the level of expression of CD137L in one or more samples isolated from one or more patients determined to be non-responsive to anti-CD137 antibody therapy (e.g., one or more samples isolated from one or more patients determined by a clinician to be non-responsive to anti-CD137 antibody therapy (such as patients receiving treatment with an anti-CD137 antibody in a clinical trial)).

In some embodiments, the reference level is a pre-determined level of CD137L expression (e.g., the average level of expression of CD137L in a database of diseased samples (such as tissue biopsies or serum samples) isolated from multiple reference patients; the average level of expression of CD137L in a database of samples (such as tissue biopsies or serum samples) isolated from multiple healthy reference patients; etc.).

In some embodiments, the reference level of expression of CD137L refers to a detectable level of expression. That is to say, in some embodiments, the level of expression of CD137L measured in the sample obtained from the subject is considered to be lower than a reference level when the level of expression of CD137L in the sample is undetectable, e.g., below the limit of detection.

In some embodiments, the level of expression of CD137L measured in the sample obtained from the subject is considered to be lower than the reference level when the level of expression of CD137L in the sample is at least about 25% lower than the reference level. For example, the level of expression of CD137L measured in the sample obtained from the subject is considered to be lower than the reference level when the level of expression of CD137L in the sample is at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% lower than the reference level. In some embodiments, the level of expression of CD137L measured in the sample obtained from the subject is considered to be lower than the reference level when the level of expression of CD137L in the sample is at least about 1-fold lower than the reference level. For example, the level of expression of CD137L measured in the sample obtained from the subject is considered to be lower than the reference level when the level of expression of CD137L in the sample is at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5 fold, at least about 8-fold, at least about 8.5-fold, at least about 9-fold, at least about 9.5-fold, at least about 10-fold, at least about 100-fold, or at least about 1000-fold lower than the reference level. In some embodiments, the level of expression of CD137L in the sample obtained from the subject is below the limit of detection. In some embodiments, the level of expression of CD137L measured in the sample obtained from the subject is considered to be lower than the reference level when the level of expression of CD137L in the sample is below the limit of detection while the reference level is above the limit of detection, is detectable, and/or is not zero. In some embodiments, a level is considered to be below the limit of detection when the level does not give an appreciable signal, a detectable signal, and/or is not significantly different than an appropriate negative control when performing an assay for measuring the level of CD137L expression (e.g., below the limit of detection of an assay measuring RNA transcript expression of CD137L (such as RT-PCR, in situ hybridization, and/or next generation sequencing), below the limit of detection of an assay measuring CD137L protein expression (such as an immunoassay, PET imaging, Western blotting, ELISA, immunohistochemistry, and/or flow cytometry), etc.).

In some embodiments, a subject is administered an effective amount of an anti-CD137 antibody when the level of expression of CD137L in a sample obtained from the subject is lower than the reference level. In some embodiments, a subject is determined to be likely to respond to an anti-CD137 antibody when the level of expression of CD137L in a sample obtained from the subject is lower than the reference level. In some embodiments, a subject is administered an effective amount of an anti-CD137 antibody after the subject has been determined to be likely to respond to the anti-CD137 antibody. In some embodiments, a subject having cancer is selected for treatment with an anti-CD137 antibody when the level of expression of CD137L in a sample obtained from the subject is lower than the reference level. In some embodiments, a subject is positively stratified for enrollment into an anti-CD137 antibody therapy when the level of expression of CD137L in a sample obtained from the subject is lower than the reference level.

In some embodiments, the level of expression of CD137L measured in the sample obtained from the subject is considered to be higher than the reference level when the level of expression of CD137L in the sample is at least about 5% higher than the reference level. For example, the level of expression of CD137L measured in the sample obtained from the subject is considered to be higher than the reference level when the level of expression of CD137L in the sample is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% higher than the reference level. In some embodiments, the level of expression of CD137L measured in the sample obtained from the subject is considered to be higher than the reference level when the level of expression of CD137L in the sample is at least about 1-fold higher than the reference level. For example, the level of expression of CD137L measured in the sample obtained from the subject is considered to be higher than the reference level when the level of expression of CD137L in the sample is at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5 fold, at least about 8-fold, at least about 8.5-fold, at least about 9-fold, at least about 9.5-fold, at least about 10-fold, at least about 100-fold, or at least about 1000-fold higher than the reference level. In some embodiments, the level of expression of CD137L in the reference sample is below the limit of detection. In some embodiments, the level of expression of CD137L measured in the sample obtained from the subject is considered to be higher than the reference level when the level of expression of CD137L in the sample is above the limit of detection, is detectable, and/or is not zero while the level of expression of CD137L in the reference sample is below the limit of detection. In some embodiments, a level is considered to be below the limit of detection when the level does not give an appreciable signal, a detectable signal, and/or is not significantly different than an appropriate negative control when performing an assay for measuring the level of CD137L expression (e.g., below the limit of detection of an assay measuring RNA transcript expression of CD137L (such as RT-PCR, in situ hybridization, and/or next generation sequencing), below the limit of detection of an assay measuring CD137L protein expression (such as an immunoassay, PET imaging, Western blotting, ELISA, immunohistochemistry, and/or flow cytometry), etc.).

In some embodiments, a subject is administered an effective amount of a checkpoint blockade immunotherapy when the level of expression of CD137L in a sample obtained from the subject is higher than the reference level. In some embodiments, a subject is determined to be likely to respond to a checkpoint blockade immunotherapy when the level of expression of CD137L in a sample obtained from the subject is higher than the reference level. In some embodiments, a subject is administered an effective amount of a checkpoint blockade immunotherapy after the subject has been determined to be likely to respond to the checkpoint blockade immunotherapy. In some embodiments, a subject having cancer is selected for treatment with a checkpoint blockade immunotherapy when the level of expression of CD137L in a sample obtained from the subject is higher than the reference level. In some embodiments, a subject is positively stratified for enrollment into a checkpoint blockade immunotherapy when the level of expression of CD137L in a sample obtained from the subject is higher than the reference level. In some embodiments, a subject is negatively stratified for enrollment into an anti-CD137 antibody therapy when the level of expression of CD137L in a sample obtained from the subject is higher than the reference level.

In some embodiments, an anti-CD137 agonist antibody activates or enhances one or more activities of CD137. In some embodiments, an anti-CD137 agonist antibody demonstrates one or more of the following properties (e.g., in an in vitro assay using a CD137-expressing cell, such as a T cell)): stimulates T cell (e.g., CD8+ T cell) proliferation, induces cytokine (e.g., IFN-γ) secretion by T cells, and induces NFκB activation (e.g., in an NFκB reporter assay).

CD137L Expression and Anti-CD137 Antibody Therapies

In some embodiments, the present disclosure relates to methods of treating or delaying progression of cancer in a subject in need thereof comprising administering an effective amount of an anti-CD137 antibody to the subject if the level of expression of CD137L in a sample obtained from the subject is lower than a reference level. In some embodiments, the method comprises obtaining a sample from the subject, and measuring the level of expression of CD137L in the sample prior to administration of the anti-CD137 antibody. In some embodiments, the level of expression of CD137L in the sample obtained from the subject is below the limit of detection. In some embodiments, the subject is administered the anti-CD137 antibody when CD137L expression is below the limit of detection. In some embodiments, the anti-CD137 antibody is any one or more of the anti-CD137 antibodies described herein.

In some embodiments, the present disclosure relates to methods of determining whether a subject is likely to respond to an anti-CD137 antibody. In some embodiments, the method comprises obtaining a sample from the subject, measuring the level of expression of CD137L in the sample, and determining that the subject is likely to respond to the anti-CD137 antibody when the level of expression of CD137L in the sample is lower than a reference level. In some embodiments, the level of expression of CD137L in the sample obtained from the subject is below the limit of detection. In some embodiments, the subject is determined to be likely to respond to the anti-CD137 antibody when the CD137L expression is below the limit of detection.

In some embodiments, the present disclosure relates to methods of treating or delaying progression of cancer in a subject in need thereof comprising administering an effective amount of an anti-CD137 antibody to the subject after it is determined that the subject is likely to respond to the anti-CD137 antibody. In some embodiments, the anti-CD137 antibody is any one or more of the anti-CD137 antibodies described herein. In some embodiments, responsiveness of the subject comprises treatment efficacy. In some embodiments, responsiveness of the subject comprises reduced tumor volume. In some embodiments, responsiveness of the subject comprises serological responsiveness.

In some embodiments, the present disclosure relates to methods of selecting a subject having cancer for treatment with an anti-CD137 antibody. In some embodiments, the method comprises measuring the level of expression of CD137L in a sample obtained from the subject, and selecting the subject for treatment with the anti-CD137 antibody if the level of expression of CD137L in the sample is lower than a reference level. In some embodiments, the level of expression of CD137L in the sample obtained from the subject is below the limit of detection. In some embodiments, the subject is selected for treatment with the anti-CD137 antibody when the CD137L expression is below the limit of detection. In some embodiments, the anti-CD137 antibody is any one or more of the anti-CD137 antibodies described herein.

Anti-CD137 Antibodies

In some embodiments, the present disclosure relates to the use of an anti-CD137 antibody. In some embodiments, the anti-CD137 antibody binds to human CD137. In some embodiments, the present disclosure provides an isolated antibody that binds to human CD137 at an epitope within amino acid residues 34-108 or 34-93 of SEQ ID NO.: 531. The antibody, in some embodiments, binds human CD137 with a K_{D} of 50 nM or less as measured by surface plasmon resonance. In certain embodiments, the antibody can be cross-reactive with at least one non-human species selected from the list consisting of cynomolgus monkey, mouse, rat and dog.

In one aspect, the present disclosure provides an isolated antibody comprising a heavy chain variable region and a light chain variable region, a) wherein the heavy chain variable region comprises an HVR-H1, an HVR-H2, and an HVR-H3, wherein the HVR-H1 comprises an amino acid sequence according to a formula selected from the group consisting of: Formula (I): X1TFX2X3YX4IHWV (SEQ ID NO:532), wherein X1 is F or Y, X2 is S or T, X3 is G, N, or S, and X4 is A, G, or W; Formula (II): YSIX1SGX2X3WX4WI (SEQ ID NO:533), wherein X1 is S or T, X2 is H or Y, X3 is H or Y, and X4 is A, D, G, N, S, or T; and Formula (III): FSLSTX1GVX2VX3WI (SEQ ID NO:534), wherein X1 is G or S, X2 is A or G, and X3 is A, G, S, or T; wherein the HVR-H2 comprises an amino acid sequence according to a formula selected from the group consisting of: Formula (IV): LALIDWX1X2DKX3YSX4SLKSRL (SEQ ID NO:535), wherein X1 is A, D, or Y, X2 is D or G, X3 is R, S, or Y, and X4 is P or T; Formula (V): IGX1IYHSGX2TYYX3PSLKSRV (SEQ ID NO:536), wherein X1 is D or E, X2 is N or S, and X3 is N or S; and Formula (VI): VSX1ISGX2GX3X4TYYADSVKGRF (SEQ ID NO:537), wherein X1 is A, G, S, V, or Y, X2 is A, D, S, or Y, X3 is D, G, or S, and X4 is S or T; and wherein the HVR-H3 comprises an amino acid sequence according to Formula (VII): ARX1GX2X3X4VX5GDWFX6Y (SEQ ID NO:538), wherein X1 is E or G, X2 is E or S, X3 is D or T, X4 is A, T, or V, X5 is A, I, L, T, or V, and X6 is A, D, or G; and/or b) wherein the light chain variable region comprises an HVR-L1, an HVR-L2, and an HVR-L3, wherein the HVR-L1 comprises an amino acid sequence according to Formula (VIII): X1ASQX2X3X4X5X6X7X8 (SEQ ID NO:539), wherein X1 is Q or R, X2 is D, G, or S, X3 is I or V, X4 is G, R, S, or T, X5 is P, R, S, or T, X6 is A, D, F, S, V, or Y, X7 is L or V, and X8 is A, G, or N; wherein the HVR-L2 comprises an amino acid sequence according to Formula (IX): X1ASX2X3X4X5GX6 (SEQ ID NO:540), wherein X1 is A or D, X2 is N, S, or T, X3 is L or R, X4 is A, E, or Q, X5 is S or T, and X6 is I or V; and wherein the HVR-L3 comprises an amino acid sequence according to a formula selected from the group consisting of: Formula (X): YCQQX1YX2X3X4T (SEQ ID NO:541), wherein X1 is A, G, S, or Y, X2 is Q, S, or Y, X3 is I, L, T, or Y, and X4 is I, S, V, or W; and Formula (XI): YCX1QX2X3X4X5PX6T (SEQ ID NO:542), wherein X1 is E or Q, X2 is P, S, or Y, X3 is D, L, S, T, or Y, X4 is D, E, H, S, or T, X5 is D, L T, or W, and X6 is L, P, R, or V.

In some embodiments, the antibody can comprise an HVR_H1 having the amino acid sequence selected from the group consisting of SEQ ID NOs: 1-60-312, an HVR_H2 having the amino acid sequence selected from the group consisting of SEQ ID NOs: 61-120, an HVR_H3 having the amino acid sequence selected from the group consisting of SEQ ID NOs: 121-180, an HVR_L1 having the amino acid sequence selected from the group consisting of SEQ ID NOs: 181-240, an HVR_L2 having the amino acid sequence selected from the group consisting of SEQ ID NOs: 241-300, and/or an HVR_L3 having the amino acid sequence selected from the group consisting of SEQ ID NOs: 301-360.

In certain embodiments, the antibody can comprise a VL and/or VH having the amino acid sequence selected from the group consisting of SEQ ID NOs: 361-480. In certain embodiments, the antibody can comprise a light chain and/or heavy chain (e.g., those of IgG such as IgG4) having the amino acid sequences selected from the group consisting of SEQ ID NOs: 481-504.

The CD137 antibodies described herein can be in any class, such as IgG, IgM, IgE, IgA, or IgD. It is preferred that the CD137 antibodies are in the IgG class, such as IgG1, IgG2, IgG3, or IgG4 subclass. A CD137 antibody can be converted from one class or subclass to another class or subclass using methods known in the art. An exemplary method for producing an antibody in a desired class or subclass comprises the steps of isolating a nucleic acid encoding a heavy chain of an CD137 antibody and a nucleic acid encoding a light chain of a CD137 antibody, isolating the sequence encoding the V_{H} region, ligating the V_{H} sequence to a sequence encoding a heavy chain constant region of the desired class or subclass, expressing the light chain gene and the heavy chain construct in a cell, and collecting the CD137 antibody.

Further, the antibodies provided by the present disclosure can be monoclonal or polyclonal, but preferably monoclonal.

Examples of specific isolated antibodies (or antigen binding fragments thereof) provided by the present disclosure include those listed in Tables 1-3. The amino acid sequences of the heavy chain variable region, full length heavy chain for the IgG2 and IgG4 subclass, light chain variable region, and full length light chain of these antibodies are also provided hereunder. In some embodiments, the anti-CD137 antibody is any anti-CD137 antibody described in PCT International Application No. PCT/CN2017/098332 (incorporated herein by reference in its entirety).

In some embodiments, the anti-CD137 antibody has a human Fc region, e.g., a human IgG1 or human IgG4 Fc region. In some embodiments, the anti-CD137 antibody has one or more effector functions, including but not limited to C1q binding, complement-dependent cytotoxicity (CDC), cross-linking mediated by FcgRIIb, Fc receptor binding (e.g., binding to FcgRIIa and/or FcgRIIb, such as FcgRIIb1 or FcgRIIb2), antibody-dependent cellular cytotoxicity (ADCC), and/or phagocytosis. In some embodiments, the anti-CD137 antibody has a human Fc region, e.g., a human IgG1 or human IgG4 Fc region, with one or more mutations (e.g., substitutions) for increasing or enhancing one or more effector functions, including but not limited to C1q binding, complement-dependent cytotoxicity (CDC), cross-linking mediated by FcgRIIb, Fc receptor binding (e.g., binding to FcgRIIa and/or FcgRIIb, such as FcgRIIb1 or FcgRIIb2), antibody-dependent cellular cytotoxicity (ADCC), and/or phagocytosis. In some embodiments, the anti-CD137 antibody has a human IgG1 Fc region that comprises mutations (e.g., substitutions) at one or more of the following residues: 5267 and L328 (numbering based on EU index according to Kabat). In some embodiments, the anti-CD137 antibody has a human IgG1 Fc region that comprises S267E and L328F substitutions (numbering based on EU index according to Kabat). In some embodiments, the anti-CD137 antibody has a human IgG1 Fc region that comprises mutations (e.g., substitutions) at one or more of the following residues: E233, G237, P238, H268, P271, and A330 (numbering based on EU index according to Kabat). In some embodiments, the anti-CD137 antibody has a human IgG1 Fc region that comprises one or more (e.g., one, two, three, four, five, or all six) of the following substitutions: E233D, G237D, P238D, H268D, P271G, and A330R (numbering based on EU index according to Kabat).

In some embodiments, the anti-CD137 antibody binds to human CD137 and has at least one (e.g., at least one, at least two, at least three, at least four, at least five, at least six, at least seven, at least eight, or all nine) of the following functional properties: (a) binds to human CD137 with a KD of 500 nM or less; (b) has agonist activity on human CD137; (c) does not bind to human OX40, CD40, GITR and/or CD27 receptor at concentration up to 1000 nM; (d) is cross-reactive with monkey (e.g., GenBank Gene ID 102127961), mouse (e.g., GenBank Gene ID 21942), rat (e.g., GenBank Gene ID 500590), or dog (e.g., GenBank Gene ID 608274) CD137; (e) does not induce ADCC effects; (f) is capable of inhibiting tumor cell growth; (g) has therapeutic effect on a cancer; (h) blocks binding between CD137 and CD137L; and (i) inhibits removal of CD137L on APCs via the trogocytic transfer of CD137 from activated T cells to APCs. In some embodiments, binding between CD137 and CD137L is measured (e.g., in vitro) by BIAcore (e.g., surface plasmon resonance or SPR), ELISA, or flow cytometry. In some embodiments, the antibody binds to cynomolgus monkey CD137. In some embodiments, the anti-CD137 antibody is an agonist antibody. In some embodiments, the anti-CD137 antibody is a human antibody. In some embodiments, the human antibody possesses an amino acid sequence which corresponds to that of an antibody produced by a human or a human cell or derived from a non-human source that utilizes human antibody repertoires or other human antibody-encoding sequences.

In some embodiments, one or more of the antibodies or antigen-binding fragments of the present disclosure inhibit or block the binding between CD137 and its ligand (e.g., human CD137 and human CD137L). In some embodiments, the antibodies or antigen-binding fragments block the binding between CD137 and its ligand in vitro. In some embodiments, the antibody or antigen-binding fragment has a half maximal inhibitory concentration (IC50) of about 500 nM or less (e.g., about 500 nM or less, about 400 nM or less, about 300 nM or less, about 200 nM or less, about 100 nM or less, about 50 nM or less, about 25 nM or less, about 10 nM or less, about 1 nM or less, etc.) for blocking binding of CD137 its ligand. In some embodiments, the antibody or antigen-binding fragment has a half maximal inhibitory concentration (IC50) of about 100 nM or less for blocking binding of CD137 its ligand. In some embodiments, the antibody or antigen-binding fragment has a half maximal inhibitory concentration (IC50) of about 10 nM or less for blocking binding of CD137 its ligand. In some embodiments, the antibody or antigen-binding fragment has a half maximal inhibitory concentration (IC50) lower than that of a reference antibody (e.g., comprising an IgG2 Fc region) comprising VH and VL domain sequences of SEQ ID NOs:545 and 546, respectively. In some embodiments, the antibody or antigen-binding fragment has a half maximal inhibitory concentration (IC50) lower than that of a reference antibody (e.g., comprising an IgG4 Fc region) comprising VH and VL domain sequences of SEQ ID NOs:547 and 548, respectively. In some embodiments, the antibody or antigen-binding fragment completely blocks binding of human CD137 to its ligand when provided at a concentration of about 100 nM or greater (e.g., about 100 nM or greater, about 500 nM or greater, about 1 μM or greater, about 10 μM or greater, etc.). As used herein, the term “complete blocking” or “completely blocks” refers to the antibody or antigen-binding fragment's ability to reduce binding between a first protein and a second protein by at least about 80% (e.g., at least about 80%, at least about 85%, at least about 90%, at least about 95%, at least about 99%, etc.). Methods of measuring the ability of an antibody or antigen-binding fragment to block binding of a first protein (e.g., a CD137) and a second protein (e.g., CD137L) are known in the art, including, without limitation, via BIAcore analysis, ELISA assays, and flow cytometry (See e.g., Example 6 below).

In some embodiments, one or more of the antibodies or antigen-binding fragments of the present disclosure inhibit or block removal of CD137L on APCs via the trogocytic transfer of CD137 from activated T cells to APCs. In some embodiments, the antibody or antigen-binding fragment inhibits or blocks removal of CD137L on APCs via the trogocytic transfer of CD137 from activated T cells to APCs more effectively than a reference antibody (e.g., comprising an IgG2 Fc region) comprising VH and VL domain sequences of SEQ ID NOs:545 and 546, respectively. In some embodiments, the antibody or antigen-binding fragment inhibits or blocks removal of CD137L on APCs via the trogocytic transfer of CD137 from activated T cells to APCs more effectively than a reference antibody (e.g., comprising an IgG4 Fc region) comprising VH and VL domain sequences of SEQ ID NOs:547 and 548, respectively. Without wishing to be bound to theory, it is thought that CD137 can be transferred by trogocytosis from one cell type to another (e.g., from activated T cells and/or tumor cells to antigen presenting cells), and if CD137L is present on the recipient cell, the transferred CD137 can bind to CD137L, forming a complex that is internalized and thereby removing surface-expressed CD137L. See, e.g., Shao, Z. et al. (2015). J. Leukocyte Biol. 97:909-919. An anti-CD137 antibody of the present disclosure can therefore prevent the removal of CD137L from cells (e.g., APCs) and its subsequent reduction in immune activity, eliminating the reverse signaling by CD137L on APCs. In some embodiments, trogocytic transfer-induced CD137L removal can be measured, e.g., by mixing cells that express CD137 (e.g., T cells or NK cells) with cells that express CD137L (e.g., APCs or tumor cells) and measuring CD137L expression.

Human CD137 is a 255 amino acid protein (GenBank Accession No. NM_001561; NP 001552; SEQ ID NO.: 531). In some embodiments, the protein comprises a signal sequence (amino acid residues 1-17), followed by an extracellular domain (169 amino acids), a transmembrane region (27 amino acids), and an intracellular domain (42 amino acids) (Cheuk ATC et al. 2004 Cancer Gene Therapy 11: 215-226).

The amino acid sequences of exemplary CD137 polypeptides are shown below:

Human CD137 polypeptide sequence:
(SEQ ID NO: 531)
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL;
Mouse CD137 polypeptide sequence:
(SEQ ID NO: 543)
MGNNCYNVVVIVLLLVGCEKVGAVQNSCDNCQPGTFCRKYNPVCKSCPPS
TFSSIGGQPNCNICRVCAGYFRFKKFCSSTHNAECECIEGFHCLGPQCTR
CEKDCRPGQELTKQGCKTCSLGTFNDQNGTGVCRPWTNCSLDGRSVLKTG
TTEKDVVCGPPVVSFSPSTTISVTPEGGPGGHSLQVLTLFLALTSALLLA
LIFITLLFSVLKW1RKKFPHIFKQPFKKTTGAAQEEDACSCRCPQEEEGG
GGGYEL;
Cynomolgus monkey CD137 polypeptide sequence:
(SEQ ID NO: 544)
MGNSCYNIVATLLLVLNFERTRSLQDLCSNCPAGTFCDNNRSQICSPCPP
NSFSSAGGQRTCDICRQCKGVFKTRKECSSTSNAECDCISGYHCLGAECS
MCEQDCKQGQELTKKGCKDCCFGTFNDQKRGICRPWTNCSLDGKSVLVNG
TKERDVVCGPSPADLSPGASSATPPAPAREPGHSPQIIFFLALTSTVVLF
LLFFLVLRFSVVKRSRKKLLYIFKQPFMRPVQTTQEEDGCSCRFPEEEEG
GCEL.

TABLE 1
HVR sequences of anti-CD137 antibodies

| AB | | HVR-H1 | HVR-H2 | HVR-H3 | HVR-L1 | HVR-L2 | HVR-L31 |
|---|---|---|---|---|---|---|---|
| ID | VH/VL | SEQ ID NO. | SEQ ID NO. | SEQ ID NO. | SEQ ID NO. | SEQ ID NO. | SEQ ID NO. |
| 3760 | VH1/VL1 | 1 | 61 | 121 | 181 | 241 | 301 |
| 4072 | VH2/VL2 | 2 | 62 | 122 | 182 | 242 | 302 |
| 7074 | VH3/VL3 | 3 | 63 | 123 | 183 | 243 | 303 |
| 4076 | VH4/VL4 | 4 | 64 | 124 | 184 | 244 | 304 |
| 4079 | VH5/VL5 | 5 | 65 | 125 | 185 | 245 | 305 |
| 4134 | VH6/VL6 | 6 | 66 | 126 | 186 | 246 | 306 |
| 4137 | VH7/VL7 | 7 | 67 | 127 | 187 | 247 | 307 |
| 4139 | VH8/VL8 | 8 | 68 | 128 | 188 | 248 | 308 |
| 4140 | VH9/VL9 | 9 | 69 | 129 | 189 | 249 | 309 |
| 4217 | VH10/VL10 | 10 | 70 | 130 | 190 | 250 | 310 |
| 5299 | VH11/VL11 | 11 | 71 | 131 | 191 | 251 | 311 |
| 5300 | VH12/VL12 | 12 | 72 | 132 | 192 | 252 | 312 |
| 5302 | VH13/VL13 | 13 | 73 | 133 | 193 | 253 | 313 |
| 5303 | VH14/VL14 | 14 | 74 | 134 | 194 | 254 | 314 |
| 5310 | VH15/VL15 | 15 | 75 | 135 | 195 | 255 | 315 |
| 5314 | VH16/VL16 | 16 | 76 | 136 | 196 | 256 | 316 |
| 5316 | VH17/VL17 | 17 | 77 | 137 | 197 | 257 | 317 |
| 5318 | VH18/VL18 | 18 | 78 | 138 | 198 | 258 | 318 |
| 5323 | VH19/VL19 | 19 | 79 | 139 | 199 | 259 | 319 |
| 5341 | VH20/VL20 | 20 | 80 | 140 | 200 | 260 | 320 |
| 5342 | VH21/VL21 | 21 | 81 | 141 | 201 | 261 | 321 |
| 5346 | VH22/VL22 | 22 | 82 | 142 | 202 | 262 | 322 |
| 5348 | VH23/VL23 | 23 | 83 | 143 | 203 | 263 | 323 |
| 5349 | VH24/VL24 | 24 | 84 | 144 | 204 | 264 | 324 |
| 5351 | VH25/VL25 | 25 | 85 | 145 | 205 | 265 | 325 |
| 5353 | VH26/VL26 | 26 | 86 | 146 | 206 | 266 | 326 |
| 5359 | VH27/VL27 | 27 | 87 | 147 | 207 | 267 | 327 |
| 5360 | VH28/VL28 | 28 | 88 | 148 | 208 | 268 | 328 |
| 5363 | VH29/VL29 | 29 | 89 | 149 | 209 | 269 | 329 |
| 5365 | VH30/VL30 | 30 | 90 | 150 | 210 | 270 | 330 |
| 5367 | VH31/VL31 | 31 | 91 | 151 | 211 | 271 | 331 |
| 5370 | VH32/VL32 | 32 | 92 | 152 | 212 | 272 | 332 |
| 5371 | VH33/VL33 | 33 | 93 | 153 | 213 | 273 | 333 |
| 5404 | VH34/VL34 | 34 | 94 | 154 | 214 | 274 | 334 |
| 5407 | VH35/VL35 | 35 | 95 | 155 | 215 | 275 | 335 |
| 5408 | VH36/VL36 | 36 | 96 | 156 | 216 | 276 | 336 |
| 5409 | VH37/VL37 | 37 | 97 | 157 | 217 | 277 | 337 |
| 5413 | VH38/VL38 | 38 | 98 | 158 | 218 | 278 | 338 |
| 5417 | VH39/VL39 | 39 | 99 | 159 | 219 | 279 | 339 |
| 7077 | VH40/VL40 | 40 | 100 | 160 | 220 | 280 | 340 |
| 7078 | VH41/VL41 | 41 | 101 | 161 | 221 | 281 | 341 |
| 7079 | VH42/VL42 | 42 | 102 | 162 | 222 | 282 | 342 |
| 7080 | VH43/VL43 | 43 | 103 | 163 | 223 | 283 | 343 |
| 7081 | VH44/VL44 | 44 | 104 | 164 | 224 | 284 | 344 |
| 7087 | VH45/VL45 | 45 | 105 | 165 | 225 | 285 | 345 |
| 7088 | VH46/VL46 | 46 | 106 | 166 | 226 | 286 | 346 |
| 7090 | VH47/VL47 | 47 | 107 | 167 | 227 | 287 | 347 |
| 7092 | VH48/VL48 | 48 | 108 | 168 | 228 | 288 | 348 |
| 7097 | VH49/VL49 | 49 | 109 | 169 | 229 | 289 | 349 |
| 7100 | VH50/VL50 | 50 | 110 | 170 | 230 | 290 | 350 |
| 7105 | VH51/VL51 | 51 | 111 | 171 | 231 | 291 | 351 |
| 7109 | VH52/VL52 | 52 | 112 | 172 | 232 | 292 | 352 |
| 7120 | VH53/VL53 | 53 | 113 | 173 | 233 | 293 | 353 |
| 7128 | VH54/VL54 | 54 | 114 | 174 | 234 | 294 | 354 |
| 7131 | VH55/VL55 | 55 | 115 | 175 | 235 | 295 | 355 |
| 7133 | VH56/VL56 | 56 | 116 | 176 | 236 | 296 | 356 |
| 7135 | VH57/VL57 | 57 | 117 | 177 | 237 | 297 | 357 |
| 7159 | VH58/VL58 | 58 | 118 | 178 | 238 | 298 | 358 |
| 7163 | VH59/VL59 | 59 | 119 | 179 | 239 | 299 | 359 |
| 7166 | VH60/VL60 | 60 | 120 | 180 | 240 | 300 | 360 |

In some embodiments, the anti-CD137 antibody comprises an HVR-H1 comprising the amino acid sequence of FSLSTSGVGVGWI (SEQ ID NO: 15), an HVR-H2 comprising the amino acid sequence of LALIDWDDDKYYSPSLKSRL (SEQ ID NO: 75), and an HVR-H3 comprising the amino acid sequence of ARGGSDTVLGDWFAY (SEQ ID NO: 135), an HVR-L1 comprising the amino acid sequence of RASQSVSPYLA (SEQ ID NO: 195), an HVR-L2 comprising the amino acid sequence of DASSLESGV (SEQ ID NO: 255), and an HVR-L3 comprising the amino acid sequence of YCQQGYSLWT (SEQ ID NO: 315). In some embodiments, the anti-CD137 antibody comprises an HVR-H1 comprising the amino acid sequence of YSITSGHYWAWI (SEQ ID NO: 25), an HVR-H2 comprising the amino acid sequence of VS SISGYGSTTYYADSVKGRF (SEQ ID NO: 85), and an HVR-H3 comprising the amino acid sequence of ARGGSDAVLGDWFAY (SEQ ID NO: 145), an HVR-L1 comprising the amino acid sequence of RASQGIGSFLA (SEQ ID NO: 205), an HVR-L2 comprising the amino acid sequence of DASNLETGV (SEQ ID NO: 265), and an HVR-L3 comprising the amino acid sequence of YCQQGYYLWT (SEQ ID NO: 325). In some embodiments, the anti-CD137 antibody comprises an HVR-H1 comprising the amino acid sequence of FSLSTGGVGVGWI (SEQ ID NO: 30), an HVR-H2 comprising the amino acid sequence of LALIDWADDKYYSPSLKSRL (SEQ ID NO: 90), and an HVR-H3 comprising the amino acid sequence of ARGGSDTVIGDWFAY (SEQ ID NO: 150), an HVR-L1 comprising the amino acid sequence of RASQSIGSYLA (SEQ ID NO: 210), an HVR-L2 comprising the amino acid sequence of DASNLETGV (SEQ ID NO: 270), and an HVR-L3 comprising the amino acid sequence of YCQQGYYLWT (SEQ ID NO: 330).

In some embodiments, the anti-CD137 antibody comprises the HVR-H1, HVR-H2, HVR-H3, HVR-L1, HVR-L2, and HVR-L3 of a single antibody shown in Table 1. For example, in some embodiments, the anti-CD137 antibody comprises an HVR-H1 comprising the amino acid sequence of SEQ ID NO: 1, an HVR-H2 comprising the amino acid sequence of SEQ ID NO: 61, and an HVR-H3 comprising the amino acid sequence of SEQ ID NO: 121, an HVR-L1 comprising the amino acid sequence of SEQ ID NO: 181, an HVR-L2 comprising the amino acid sequence of SEQ ID NO: 241, and an HVR-L3 comprising the amino acid sequence of SEQ ID NO: 301, as shown for antibody 3760 in Table 1; or an HVR-H1 comprising the amino acid sequence of SEQ ID NO: 2, an HVR-H2 comprising the amino acid sequence of SEQ ID NO: 62, and an HVR-H3 comprising the amino acid sequence of SEQ ID NO: 122, an HVR-L1 comprising the amino acid sequence of SEQ ID NO: 182, an HVR-L2 comprising the amino acid sequence of SEQ ID NO: 242, and an HVR-L3 comprising the amino acid sequence of SEQ ID NO: 302, as shown for antibody 4072 in Table 1.

TABLE 2
VH and VL sequences of anti-CD137 antibodies

| Hit ID | VH and VL ID | SEQ ID NO. |
|---|---|---|
| 3760 | VH1 | 361 |
| | VL1 | 421 |
| 4072 | VH2 | 362 |
| | VL2 | 422 |
| 4074 | VH3 | 363 |
| | VL3 | 423 |
| 4076 | VH4 | 364 |
| | VL4 | 424 |
| 4079 | VH5 | 365 |
| | VL5 | 425 |
| 4134 | VH6 | 366 |
| | VL6 | 426 |
| 4137 | VH7 | 367 |
| | VL7 | 427 |
| 4139 | VH8 | 368 |
| | VL8 | 428 |
| 4140 | VH9 | 369 |
| | VL9 | 429 |
| 4217 | VH10 | 370 |
| | VL10 | 430 |
| 5299 | VH11 | 371 |
| | VL11 | 431 |
| 5300 | VH12 | 372 |
| | VL12 | 432 |
| 5302 | VH13 | 373 |
| | VL13 | 433 |
| 5303 | VH14 | 374 |
| | VL14 | 434 |
| 5310 | VH15 | 375 |
| | VL15 | 435 |
| 5314 | VH16 | 376 |
| | VL16 | 436 |
| 5316 | VH17 | 377 |
| | VL17 | 437 |
| 5318 | VH18 | 378 |
| | VL28 | 438 |
| 5323 | VH19 | 379 |
| | VL19 | 439 |
| 5341 | VH20 | 380 |
| | VL20 | 440 |
| 5342 | VH21 | 381 |
| | VL21 | 441 |
| 5346 | VH22 | 382 |
| | VL22 | 442 |
| 5348 | VH23 | 383 |
| | VL23 | 443 |
| 5349 | VH24 | 384 |
| | VL24 | 444 |
| 5351 | VH25 | 385 |
| | VL25 | 445 |
| 5353 | VH26 | 386 |
| | VL26 | 446 |
| 5359 | VH27 | 387 |
| | VL27 | 447 |
| 5360 | VH28 | 388 |
| | VL28 | 448 |
| 5363 | VH29 | 389 |
| | VL29 | 449 |
| 5365 | VH30 | 390 |
| | VL30 | 450 |
| 5367 | VH31 | 391 |
| | VL31 | 451 |
| 5370 | VH32 | 392 |
| | VL32 | 452 |
| 5371 | VH33 | 393 |
| | VL33 | 453 |
| 5404 | VH34 | 394 |
| | VL34 | 454 |
| 5407 | VH35 | 395 |
| | VL35 | 455 |
| 5408 | VH36 | 396 |
| | VL36 | 456 |
| 5409 | VH37 | 397 |
| | VL37 | 457 |
| 5413 | VH38 | 398 |
| | VL38 | 458 |
| 5417 | VH39 | 399 |
| | VL39 | 459 |
| 7077 | VH40 | 400 |
| | VL40 | 460 |
| 7078 | VH41 | 401 |
| | VL41 | 461 |
| 7079 | VH42 | 402 |
| | VL42 | 462 |
| 7080 | VH43 | 403 |
| | VL43 | 463 |
| 7081 | VH44 | 404 |
| | VL44 | 464 |
| 7087 | VH45 | 405 |
| | VL45 | 465 |
| 7088 | VH46 | 406 |
| | VL46 | 466 |
| 7090 | VH47 | 407 |
| | VL47 | 467 |
| 7092 | VH48 | 408 |
| | VL48 | 468 |
| 7097 | VH49 | 409 |
| | VL49 | 469 |
| 7100 | VH50 | 410 |
| | VL50 | 470 |
| 7105 | VH51 | 411 |
| | VL51 | 471 |
| 7109 | VH52 | 412 |
| | VL52 | 472 |
| 7120 | VH53 | 413 |
| | VL53 | 473 |
| 7128 | VH54 | 414 |
| | VL54 | 474 |
| 7131 | VH55 | 415 |
| | VL55 | 475 |
| 7133 | VH56 | 416 |
| | VL56 | 476 |
| 7135 | VH57 | 417 |
| | VL57 | 477 |
| 7159 | VH58 | 418 |
| | VH58 | 478 |
| 7163 | VH59 | 419 |
| | VL59 | 479 |
| 7166 | VH60 | 420 |
| | VL60 | 480 |
| AC1097 | HC25 | 545 |
| | LC25 | 546 |
| AC1121 | HC26 | 547 |
| | LC26 | 548 |

In some embodiments, the anti-CD137 antibody comprises a heavy chain variable region comprising the amino acid sequence of: EVQLVESGGGLVQPGGSLRLSCAASGFSLSTSGVGVGWIRQAPGKGLEWLALIDWDDD KYYSPSLKSRLTISRDNSKNTLYLQLNSLRAEDTAVYYCARGGSDTVLGDWFAYWGQG TLVTVSS (SEQ ID NO: 375), and a light chain variable region comprising the amino acid sequence of: DIQLTQSPSSLSASVGDRVTITCRASQSVSPYLAWYQQKPGKAPKLLIYDASSLESGVPSR FSGSGSGTDFTLTISSLQPEDFATYYCQQGYSLWTFGQGTKVEIKR (SEQ ID NO: 435). In some embodiments, the anti-CD137 antibody comprises a heavy chain variable region comprising the amino acid sequence of: EVQLVESGGGLVQPGGSLRLSCAASGYSITSGHYWAWIRQAPGKGLEWVSSISGYGSTT YYADSVKGRFTISRDNSKNTLYLQLNSLRAEDTAVYYCARGGSDAVLGDWFAYWGQG TLVTVSS (SEQ ID NO: 385), and a light chain variable region comprising the amino acid sequence of: DIQLTQSPSSLSASVGDRVTITCRASQGIGSFLAWYQQKPGKAPKLLIYDASNLETGVPSR FSGSGSGTDFTLTISSLQPEDFATYYCQQGYYLWTFGQGTKVEIKR (SEQ ID NO: 445). In some embodiments, the anti-CD137 antibody comprises a heavy chain variable region comprising the amino acid sequence of: EVQLVESGGGLVQPGGSLRLSCAASGFSLSTGGVGVGWIRQAPGKGLEWLALIDWADD KYYSPSLKSRLTISRDNSKNTLYLQLNSLRAEDTAVYYCARGGSDTVIGDWFAYWGQG TLVTVSS (SEQ ID NO: 390), and a light chain variable region comprising the amino acid sequence of: DIQLTQSPSSLSASVGDRVTITCRASQSIGSYLAWYQQKPGKAPKWYDASNLETGVPSR FSGSGSGTDFTLTISSLQPEDFATYYCQQGYYLWTFGQGTKVEIKR (SEQ ID NO: 450).

In some embodiments, the anti-CD137 antibody comprises the VH and VL domain of a single antibody shown in Table 2. For example, in some embodiments, the anti-CD137 antibody comprises a heavy chain variable region comprising the amino acid sequence of SEQ ID NO:361 and a light chain variable region comprising the amino acid sequence of SEQ ID NO:421, as shown for antibody 3760 in Table 2; or a heavy chain variable region comprising the amino acid sequence of SEQ ID NO:362 and a light chain variable region comprising the amino acid sequence of SEQ ID NO:422, as shown for antibody 4072 in Table 2.

TABLE 3
Heavy chain and light chain sequences of anti-CD137 antibodies

| | | Heavy chain | |
|---|---|---|---|
| | | (HC) and Light | |
| Hit ID | IgG ID | Chain (LC) ID | SEQ ID NO. |
| 4072 | AG10054 | HC1 | 481 |
| | | LC 1 | 505 |
| 5303 | AG10057 | HC 2 | 482 |
| | | LC 2 | 506 |
| 5310 | AG10058 | HC 3 | 483 |
| | | LC 3 | 507 |
| 5351 | AG10059 | HC 4 | 484 |
| | | LC 4 | 508 |
| 5359 | AG10060 | HC 5 | 485 |
| | | LC 5 | 509 |
| 5370 | AG10061 | HC 6 | 486 |
| | | LC 6 | 510 |
| 5404 | AG10062 | HC 7 | 487 |
| | | LC 7 | 511 |
| 5413 | AG10063 | HC 8 | 488 |
| | | LC 8 | 512 |
| 4074 | AG10079 | HC 9 | 489 |
| | | LC 9 | 513 |
| 4217 | AG10080 | HC 10 | 490 |
| | | LC 10 | 514 |
| 5299 | AG10081 | HC 11 | 491 |
| | | LC 11 | 515 |
| 5300 | AG10082 | HC 12 | 492 |
| | | LC 12 | 516 |
| 5323 | AG10083 | HC 13 | 493 |
| | | LC 13 | 517 |
| 5360 | AG10084 | HC 14 | 494 |
| | | LC 14 | 518 |
| 5367 | AG10085 | HC 15 | 495 |
| | | LC 15 | 519 |
| 5409 | AG10086 | HC 16 | 496 |
| | | LC 16 | 520 |
| 5302 | AG10124 | HC 17 | 497 |
| | | LC 17 | 521 |
| 5314 | AG10125 | HC 18 | 498 |
| | | LC 18 | 522 |
| 5316 | AG10126 | HC 19 | 499 |
| | | LC 19 | 523 |
| 5318 | AG10127 | HC20 | 500 |
| | | LC 20 | 524 |
| 5342 | AG10128 | HC 21 | 501 |
| | | LC 21 | 525 |
| 5353 | AG10129 | HC 22 | 502 |
| | | LC 22 | 526 |
| 5365 | AG10131 | HC 23 | 503 |
| | (αCD137-AB1) | LC 23 | 527 |
| 5408 | AG10132 | HC 24 | 504 |
| | | LC24 | 528 |

In some embodiments, the anti-CD137 antibody comprises a heavy chain comprising the amino acid sequence of: EVQLVESGGGLVQPGGSLRLSCAASGFSLSTS GVGVGWIRQAPGKGLEWLALIDWDDD KYYSPSLKSRLTISRDNSKNTLYLQLNSLRAEDTAVYYCARGGSDTVLGDWFAYWGQG TLVTVSSASTKGPSVFPLAPCSRSTSESTAALGCLVKDYFPEPVTVSWNSGALTSGVHTF PAVLQSSGLYSLSSVVTVPSSSLGTKTYTCNVDHKPSNTKVDKRVESKYGPPCPPCPAPE FLGGPSVFLFPPKPKDTLMISRTPEVTCVVVDVSQEDPEVQFNWYVDGVEVHNAKTKPR EEQFNSTYRVVSVLTVLHQDWLNGKEYKCKVSNKGLPS SIEKTISKAKGQPREPQVYTL PPSQEEMTKNQVSLTCLVKGFYPSDIAVEWESNGQPENNYKTTPPVLDSDGSFFLYSRLT VDKSRWQEGNVFSCSVMHEALHNHYTQKSLSLSLGK (SEQ ID NO: 483), and a light chain comprising the amino acid sequence of: DIQLTQSPSSLSASVGDRVTITCRASQSVSPYLAWYQQKPGKAPKLLIYDASSLESGVPSR FSGSGSGTDFTLTISSLQPEDFATYYCQQGYSLWTFGQGTKVEIKRTVAAPSVFIFPPSDE QLKSGTASVVCLLNNFYPREAKVQWKVDNALQSGNSQESVTEQDSKDSTYSLSSTLTLS KADYEKHKVYACEVTHQGLSSPVTKSFNRGEC (SEQ ID NO: 507). In some embodiments, the anti-CD137 antibody comprises a heavy chain comprising the amino acid sequence of: EVQLVESGGGLVQPGGSLRLSCAASGYSITSGHYWAWIRQAPGKGLEWVSSISGYGSTT YYADSVKGRFTISRDNSKNTLYLQLNSLRAEDTAVYYCARGGSDAVLGDWFAYWGQG TLVTVSSASTKGPSVFPLAPCSRSTSESTAALGCLVKDYFPEPVTVSWNSGALTSGVHTF PAVLQSSGLYSLSSVVTVPSSSLGTKTYTCNVDHKPSNTKVDKRVESKYGPPCPPCPAPE FLGGPSVFLFPPKPKDTLMISRTPEVTCVVVDVSQEDPEVQFNWYVDGVEVHNAKTKPR EEQFNSTYRVVSVLTVLHQDWLNGKEYKCKVSNKGLPSSIEKTISKAKGQPREPQVYTL PPSQEEMTKNQVSLTCLVKGFYPSDIAVEWESNGQPENNYKTTPPVLDSDGSFFLYSRLT VDKSRWQEGNVFSCSVMHEALHNHYTQKSLSLSLGK (SEQ ID NO: 484), and a light chain comprising the amino acid sequence of: DIQLTQSPSSLSASVGDRVTITCRASQGIGSFLAWYQQKPGKAPKLLIYDASNLETGVPSR FSGSGSGTDFTLTISSLQPEDFATYYCQQGYYLWTFGQGTKVEIKRTVAAPSVFIFPPSDE QLKSGTASVVCLLNNFYPREAKVQWKVDNALQSGNSQESVTEQDSKDSTYSLSSTLTLS KADYEKHKVYACEVTHQGLSSPVTKSFNRGEC (SEQ ID NO: 508). In some embodiments, the anti-CD137 antibody comprises a heavy chain comprising the amino acid sequence of: EVQLVESGGGLVQPGGSLRLSCAASGFSLSTGGVGVGWIRQAPGKGLEWLALIDWADD KYYSPSLKSRLTISRDNSKNTLYLQLNSLRAEDTAVYYCARGGSDTVIGDWFAYWGQG TLVTVSSASTKGPSVFPLAPCSRSTSESTAALGCLVKDYFPEPVTVSWNSGALTSGVHTF PAVLQSSGLYSLSSVVTVPSSSLGTKTYTCNVDHKPSNTKVDKRVESKYGPPCPPCPAPE FLGGPSVFLFPPKPKDTLMISRTPEVTCVVVDVSQEDPEVQFNWYVDGVEVHNAKTKPR EEQFNSTYRVVSVLTVLHQDWLNGKEYKCKVSNKGLPSSIEKTISKAKGQPREPQVYTL PPSQEEMTKNQVSLTCLVKGFYPSDIAVEWESNGQPENNYKTTPPVLDSDGSFFLYSRLT VDKSRWQEGNVFSCSVMHEALHNHYTQKSLSLSLGK (SEQ ID NO: 503), and a light chain comprising the amino acid sequence of: DIQLTQSPSSLSASVGDRVTITCRASQSIGSYLAWYQQKPGKAPKLLIYDASNLETGVPSR FSGSGSGTDFTLTISSLQPEDFATYYCQQGYYLWTFGQGTKVEIKRTVAAPSVFIFPPSDE QLKSGTASVVCLLNNFYPREAKVQWKVDNALQSGNSQESVTEQDSKDSTYSLSSTLTLS KADYEKHKVYACEVTHQGLSSPVTKSFNRGEC (SEQ ID NO: 527).

In some embodiments, the anti-CD137 antibody comprises the heavy chain and light chain of a single antibody shown in Table 3. For example, in some embodiments, the anti-CD137 antibody comprises a heavy chain comprising the amino acid sequence of SEQ ID NO:481 and a light chain comprising the amino acid sequence of SEQ ID NO:505, as shown for antibody 4072 in Table 3; or a heavy chain comprising the amino acid sequence of SEQ ID NO:482 and a light chain variable region comprising the amino acid sequence of SEQ ID NO:506, as shown for antibody 5303 in Table 3.

### Antigen Binding Fragments

In some aspects, the present disclosure provides antigen-binding fragments of any of the CD137 antibodies provided by the present disclosure.

The antigen-binding fragment may comprise any sequences of the antibody. In some embodiments, the antigen-binding fragment comprises the amino acid sequence of: (1) a light chain of a CD137 antibody; (2) a heavy chain of a CD137 antibody; (3) a variable region from the light chain of a CD137 antibody; (4) a variable region from the heavy chain of a CD137 antibody; (5) one or more HVRs (two, three, four, five, or six HRVs) of a CD137 antibody; or (6) three HVRs from the light chain and three HVRs from the heavy chain of a CD137 antibody.

In some particular embodiments, the disclosure provides an antigen-binding fragment of an antibody selected from those listed in Tables 1-3.

In some other particular embodiments, the antigen-binding fragments of an CD137 antibody include: (i) a Fab fragment, which is a monovalent fragment consisting of the V_{L}, V_{H}, C_{L} and C_{H}1 domains; (ii) a F(ab′)₂ fragment, which is a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody; (v) a dAb fragment (Ward et al., (1989) Nature 341:544-546), which consists of a V_{H} domain; (vi) an isolated CDR, and (vii) single chain antibody (scFv), which is a polypeptide comprising a V_{L} region of an antibody linked to a V_{H} region of an antibody. Bird et al., (1988) Science 242:423-426 and Huston et al., (1988) Proc. Natl. Acad. Sci. USA 85:5879-5883.

In some particular embodiments, the antigen-binding fragment is a Fab fragment selected from those listed in Table 1.

### Antibody Derivatives

The anti-CD137 antibodies as described herein may include any antibody derived from the anti-CD137 antibodies of the present disclosure.

In some further aspects, the present disclosure provides derivatives of any of the CD137 antibodies provided by the present disclosure.

In one aspect, the antibody derivative is derived from modifications of the amino acid sequences of an illustrative antibody (“parent antibody”) of the disclosure while conserving the overall molecular structure of the parent antibody amino acid sequence. Amino acid sequences of any regions of the parent antibody chains may be modified, such as framework regions, HVR regions, or constant regions. Types of modifications include substitutions, insertions, deletions, or combinations thereof, of one or more amino acids of the parent antibody.

In some embodiments, the antibody derivative comprises a V_{L}, or V_{H} region that is at least 65%, at least 75%, at least 85%, at least 90%, at least 95%, at least 96%, at least 97%, at least 98%, or at least 99% identical to an amino acid sequence as set forth in any of SEQ ID NOs: 361-480. In some embodiments, the antibody derivative comprises an HVR_H1 amino acid sequence region that is at least 65%, at least 75%, at least 85%, at least 90%, at least 95%, at least 96%, at least 97%, at least 98%, or at least 99% identical to an amino acid sequence as set forth in any of SEQ ID NOs: 1-60. In some embodiments, the antibody derivative comprises an HVR_H2 amino acid sequence region that is at least 65%, at least 75%, at least 85%, at least 90%, at least 95%, at least 96%, at least 97%, at least 98%, or at least 99% identical to an amino acid sequence as set forth in any of SEQ ID NOs: 61-120. In some embodiments, the antibody derivative comprises an HVR_H3 amino acid sequence region that is at least 65%, at least 75%, at least 85%, at least 90%, at least 95%, at least 96%, at least 97%, at least 98%, or at least 99% identical to an amino acid sequence as set forth in any of SEQ ID NOs: 121-180. In some embodiments, the antibody derivative comprises an HVR_L1 amino acid sequence for any hit shown in Tables 1-2, which can be found in SEQ ID NOs: 181-240. In some embodiments, the antibody derivative comprises an HVR_L2 amino acid sequence for any hit shown in Tables 1-2, which can be found in SEQ ID NOs: 241-300.

In some embodiments, the antibody derivative comprises an HVR_L3 amino acid sequence for any hit shown in Tables 1-2, which can be found in SEQ ID NOs: 301-360. In some particular embodiments, the derivative comprises 1, 2, 3, 4, 5, 6, 7, 8, 9, 10, 11, 12, 13, 14, or 15 conservative or non-conservative substitutions, and/or 1, 2, 3, 4, 5, 6, 7, 8, 9, 10, 11, 12, 13, 14, or 15 additions and/or deletions to an amino acid sequence as set forth in any of SEQ ID NOs: 361-480.

In some embodiments, the antibody derivative comprises a light chain or heavy chain that is at least 65%, at least 75%, at least 85%, at least 90%, at least 95%, at least 96%, at least 97%, at least 98%, or at least 99% identical to an amino acid sequence as set forth in any of SEQ ID NOs: 481-528.

Amino acid substitutions encompass both conservative substitutions and non-conservative substitutions. The term “conservative amino acid substitution” means a replacement of one amino acid with another amino acid where the two amino acids have similarity in certain physico-chemical properties such as polarity, charge, solubility, hydrophobicity, hydrophilicity, and/or the amphipathic nature of the residues involved. For example, substitutions typically may be made within each of the following groups: (a) nonpolar (hydrophobic) amino acids, such as alanine, leucine, isoleucine, valine, proline, phenylalanine, tryptophan, and methionine; (b) polar neutral amino acids, such as glycine, serine, threonine, cysteine, tyrosine, asparagine, and glutamine; (c) positively charged (basic) amino acids, such as arginine, lysine, and histidine; and (d) negatively charged (acidic) amino acids, such as aspartic acid and glutamic acid.

The modifications may be made in any positions of the amino acid sequences of the antibody, including the HVRs, framework regions, or constant regions. In one embodiment, the present disclosure provides an antibody derivative that contains the V_{H} and V_{L}, HVR sequences of an illustrative antibody of this disclosure, yet contains framework sequences different from those of the illustrative antibody. Such framework sequences can be obtained from public DNA databases or published references that include germline antibody gene sequences. For example, germline DNA sequences for human heavy and light chain variable region genes can be found in the Genbank database or in the “VBase” human germline sequence database (Kabat, E. A., et al., Sequences of Proteins of Immunological Interest, Fifth Edition, U.S. Department of Health and Human Services, NIH Publication No. 91-3242 (1991); Tomlinson, I. M., et al., J. Mol. Biol. 227:776-798 (1992); and Cox, J. P. L. et al., Eur. J. Immunol. 24:827-836 (1994)). Framework sequences that may be used in constructing an antibody derivative include those that are structurally similar to the framework sequences used by illustrative antibodies of the disclosure, e.g., similar to the V_{H} 3-23 framework sequences and/or the V_{L} λ3 or λ1-13 framework sequences used by illustrative antibodies of the disclosure. For example, the HVR_H1, HVR_H2, and HVR_H3 sequences, and the HVR_L1, HVR_L2, and HVR_L3 sequences of an illustrative antibody can be grafted onto framework regions that have the identical sequence as that found in the germline immunoglobulin gene from which the framework sequence derive, or the HVR sequences can be grafted onto framework regions that contain one or more mutations as compared to the germline sequences.

In a particular embodiment, the antibody derivative is a chimeric antibody which comprises an amino acid sequence of an illustrative antibody of the disclosure. In one example, one or more HVRs from one or more illustrative human antibodies are combined with HVRs from an antibody from a non-human animal, such as mouse or rat. In another example, all of the HVRs of the chimeric antibody are derived from one or more illustrative antibodies. In some particular embodiments, the chimeric antibody comprises one, two, or three HVRs from the heavy chain variable region or from the light chain variable region of an illustrative antibody. Chimeric antibodies can be generated using conventional methods known in the art.

Another type of modification is to mutate amino acid residues within the HRV regions of the V_{H} and/or V_{L}, chain. Site-directed mutagenesis or PCR-mediated mutagenesis can be performed to introduce the mutation(s) and the effect on antibody binding, or other functional property of interest, can be evaluated in in vitro or in vivo assays known in the art. Typically, conservative substitutions are introduced. The mutations may be amino acid additions and/or deletions. Moreover, typically no more than one, two, three, four or five residues within a HVR region are altered. In some embodiments, the antibody derivative comprises 1, 2, 3, or 4 amino acid substitutions in the heavy chain HVRs and/or in the light chain HVRs. In another embodiment, the amino acid substitution is to change one or more cysteines in an antibody to another residue, such as, without limitation, alanine or serine. The cysteine may be a canonical or non-canonical cysteine. In one embodiment, the antibody derivative has 1, 2, 3, or 4 conservative amino acid substitutions in the heavy chain HVR regions relative to the amino acid sequences of an illustrative antibody.

Modifications may also be made to the framework residues within the V_{H} and/or V_{L} regions. Typically, such framework variants are made to decrease the immunogenicity of the antibody. One approach is to “back mutate” one or more framework residues to the corresponding germline sequence. An antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back mutated” to the germline sequence by, for example, site-directed mutagenesis or PCR-mediated mutagenesis.

In addition, modifications may also be made within the Fc region of an illustrative antibody, typically to alter one or more functional properties of the antibody, such as serum half-life, complement fixation, Fc receptor binding, and/or antigen-dependent cellular cytotoxicity. In one example, the hinge region of CH1 is modified such that the number of cysteine residues in the hinge region is altered, e.g., increased or decreased. This approach is described further in U.S. Pat. No. 5,677,425. The number of cysteine residues in the hinge region of CH1 is altered to, for example, facilitate assembly of the light and heavy chains or to increase or decrease the stability of the antibody. In another case, the Fc hinge region of an antibody is mutated to decrease the biological half-life of the antibody.

Furthermore, an antibody of the disclosure may be modified to alter its potential glycosylation site or pattern in accordance with routine experimentation known in the art. In another aspect, the present disclosure provide an derivative of an CD137 antibody of the disclosure that contains at least one mutation in an variable region of a light chain or heavy chain that changes the pattern of glycosylation in the variable region. Such an antibody derivative may have an increased affinity and/or a modified specificity for binding an antigen. The mutations may add a novel glycosylation site in the V region, change the location of one or more V region glycosylation site(s), or remove a pre-existing V region glycosylation site. In one embodiment, the present disclosure provides a derivative of a CD137 antibody having a potential N-linked glycosylation site at asparagine in the heavy chain variable region, wherein the potential N-linked glycosylation site in one heavy chain variable region is removed. In another embodiment, the present disclosure provides a derivative of a CD137 antibody having a potential N-linked glycosylation site at asparagine in the heavy chain variable region, wherein the potential N-linked glycosylation site in both heavy chain variable regions is removed. Method of altering the glycosylation pattern of an antibody is known in the art, such as those described in U.S. Pat. No. 6,933,368, the disclosure of which incorporated herein by reference.

In another aspect, the present disclosure provides an antibody derivative that comprises a CD137 antibody, or antigen-binding fragment thereof, as described herein, linked to an additional molecular entity. Examples of additional molecular entities include pharmaceutical agents, peptides or proteins, detection agent or labels, and antibodies.

In some embodiments, the antibody derivative comprises an antibody of the disclosure linked to a pharmaceutical agent. Examples of pharmaceutical agents include cytotoxic agents or other cancer therapeutic agents, and radioactive isotopes. Specific examples of cytotoxic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Examples of radioactive isotopes that can be conjugated to antibodies for use diagnostically or therapeutically include, but are not limited to, iodine¹³¹, indium¹¹¹, yttrium⁹⁰ and lutetium¹⁷⁷. Methods for linking an antibody to a pharmaceutical agent are known in the art, such as using various linker technologies. Examples of linker types include hydrazones, thioethers, esters, disulfides and peptide-containing linkers. For further discussion of linkers and methods for linking therapeutic agents to antibodies, see also Saito et al., Adv. Drug Deliv. Rev. 55:199-215 (2003); Trail, et al., Cancer Immunol. Immunother. 52:328-337 (2003); Payne, Cancer Cell 3:207-212 (2003); Allen, Nat. Rev. Cancer 2:750-763 (2002); Pastan, I. and Kreitman, Curr. Opin. Investig. Drugs 3:1089-1091 (2002); Senter, P. D. and Springer, C. J. (2001) Adv. Drug Deliv. Rev. 53:247-264.

In a particular embodiment, the antibody derivative is a CD137 antibody multimer, which is a multimeric form of a CD137 antibody, such as antibody dimers, trimers, or higher-order multimers of monomeric antibodies. Individual monomers within an antibody multimer may be identical or different. In addition, individual antibodies within a multimer may have the same or different binding specificities. Multimerization of antibodies may be accomplished through natural aggregation of antibodies. For example, some percentage of purified antibody preparations (e.g., purified IgG4 molecules) spontaneously form protein aggregates containing antibody homodimers, and other higher-order antibody multimers. Alternatively, antibody homodimers may be formed through chemical linkage techniques known in the art, such as through using crosslinking agents. Suitable crosslinkers include those that are heterobifunctional, having two distinctly reactive groups separated by an appropriate spacer (such as m-maleimidobenzoyl-N-hydroxysuccinimide ester, succinimidyl 4-(maleimidomethyl)cyclohexane-1-carboxylate, and N-succinimidyl S-acethylthio-acetate) or homobifunctional (such as disuccinimidyl suberate). Such linkers are commercially available from, for example, Pierce Chemical Company, Rockford, Ill. Antibodies can also be made to multimerize through recombinant DNA techniques known in the art.

Examples of other antibody derivatives provided by the present disclosure include single chain antibodies, diabodies, domain antibodies, nanobodies, and unibodies. A “single-chain antibody” (scFv) consists of a single polypeptide chain comprising a V_{L} domain linked to a V_{H} domain wherein V_{L} domain and V_{H} domain are paired to form a monovalent molecule. Single chain antibody can be prepared according to method known in the art (see, for example, Bird et al., (1988) Science 242:423-426 and Huston et al., (1988) Proc. Natl. Acad. Sci. USA 85:5879-5883). A “diabody” consists of two chains, each chain comprising a heavy chain variable region connected to a light chain variable region on the same polypeptide chain connected by a short peptide linker, wherein the two regions on the same chain do not pair with each other but with complementary domains on the other chain to form a bispecific molecule. Methods of preparing diabodies are known in the art (See, e.g., Holliger P. et al., (1993) Proc. Natl. Acad. Sci. USA 90:6444-6448, and Poljak R. J. et al., (1994) Structure 2:1121-1123). Domain antibodies (dAbs) are small functional binding units of antibodies, corresponding to the variable regions of either the heavy or light chains of antibodies. Domain antibodies are well expressed in bacterial, yeast, and mammalian cell systems. Further details of domain antibodies and methods of production thereof are known in the art (see, for example, U.S. Pat. Nos. 6,291,158; 6,582,915; 6,593,081; 6,172,197; 6,696,245; European Patents 0368684 & 0616640; WO05/035572, WO04/101790, WO04/081026, WO04/058821, WO04/003019 and WO03/002609). Nanobodies are derived from the heavy chains of an antibody. A nanobody typically comprises a single variable domain and two constant domains (CH2 and CH3) and retains antigen-binding capacity of the original antibody. Nanobodies can be prepared by methods known in the art (See e.g., U.S. Pat. Nos. 6,765,087, 6,838,254, WO 06/079372). Unibodies consist of one light chain and one heavy chain of an IgG4 antibody. Unibodies may be made by the removal of the hinge region of IgG4 antibodies. Further details of unibodies and methods of preparing them may be found in WO2007/059782.

Antibody Production

Antibodies of the present disclosure may be produced using recombinant methods and compositions, e.g., as described in U.S. Pat. No. 4,816,567. In some embodiments, isolated nucleic acids encoding any antibody described herein are provided. Such nucleic acids may encode an amino acid sequence comprising the V_{L} and/or an amino acid sequence comprising the V_{H} of the antibodies (e.g., the light and/or heavy chains of the antibodies). In some embodiments, one or more vectors (e.g., expression vectors) comprising such nucleic acids are provided herein. In some embodiments, a host cell comprising such nucleic acids is provided. In one such embodiment, a host cell comprises (e.g., has been transformed with): (1) a vector comprising a nucleic acid that encodes an amino acid sequence comprising the V_{L}, of the antibody and an amino acid sequence comprising the V_{H} of the antibody, or (2) a first vector comprising a nucleic acid that encodes an amino acid sequence comprising the V_{L} of the antibody and a second vector comprising a nucleic acid that encodes an amino acid sequence comprising the V_{H} of the antibody. In some embodiments, the host cell is eukaryotic, e.g. a Chinese Hamster Ovary (CHO) cell or a lymphoid cell (e.g., Y0, NS0, Sp20 cells). In some embodiments, antibodies of the present disclosure are produced in CHO cells. In some embodiments, antibodies of the present disclosure are modified, and do not include a C-terminal lysine residue (e.g., the C-terminal lysine residue of an antibody heavy chain described herein is removed (such as before or during antibody production)). For example, an antibody of the present disclosure may include an antibody heavy chain having the amino acid sequence of SEQ ID NO: 503, or may include an antibody heavy chain having an amino acid sequence identical to SEQ ID NO: 503 except for the amino acid sequence lacks the C-terminal lysine residue at position 450 of SEQ ID NO: 503. In some embodiments, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of antibodies of the present disclosure, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and may be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BEM); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Hybridoma production is a very well-established procedure. The common animal system for preparing hybridomas is the murine system. Immunization protocols and techniques for isolation of immunized splenocytes for fusion are known in the art. Fusion partners (e.g., murine myeloma cells) and fusion procedures are also known. One well-known method that may be used for making antibodies provided by the present disclosure involves the use of a XenoMouse™ animal system. XenoMouse™ mice are engineered mouse strains that comprise large fragments of human immunoglobulin heavy chain and light chain loci and are deficient in mouse antibody production. See, e.g., Green et al., Nature Genetics 7:13-21 (1994) and WO2003/040170. For example, the animal is immunized with a CD137 antigen. The CD137 antigen is isolated and/or purified CD137, preferably CD137. It may be a fragment of CD137, such as the extracellular domain of CD137, particularly a CD137 extracellular domain fragment comprising amino acid resides 34-108 or 34-93 of SEQ ID NO: 531. Immunization of animals can be carried out by any method known in the art. See, e.g., Harlow and Lane, Antibodies: A Laboratory Manual, New York: Cold Spring Harbor Press, 1990. Methods for immunizing non-human animals such as mice, rats, sheep, goats, pigs, cattle and horses are well known in the art. See, e.g., Harlow and Lane, supra, and U.S. Pat. No. 5,994,619. The CD137 antigen may be administered with an adjuvant to stimulate the immune response. Exemplary adjuvants include complete or incomplete Freund's adjuvant, RIBI (muramyl dipeptides) or ISCOM (immunostimulating complexes). After immunization of an animal with a CD137 antigen, antibody-producing immortalized cell lines are prepared from cells isolated from the immunized animal. After immunization, the animal is sacrificed and lymph node and/or splenic B cells are immortalized. Methods of immortalizing cells include, but are not limited to, transferring them with oncogenes, inflecting them with the oncogenic virus cultivating them under conditions that select for immortalized cells, subjecting them to carcinogenic or mutating compounds, fusing them with an immortalized cell, e.g., a myeloma cell, and inactivating a tumor suppressor gene. See, e.g., Harlow and Lane, supra. If fusion with myeloma cells is used, the myeloma cells preferably do not secrete immunoglobulin polypeptides (a non-secretory cell line). Immortalized cells are screened using CD137, a portion thereof, or a cell expressing CD137. CD137 antibody-producing cells, e.g., hybridomas, are selected, cloned and further screened for desirable characteristics, including robust growth, high antibody production and desirable antibody characteristics, as discussed further below. Hybridomas can be expanded in vivo in syngeneic animals, in animals that lack an immune system, e.g., nude mice, or in cell culture in vitro. Methods of selecting, cloning and expanding hybridomas are well known to those of ordinary skill in the art.

CD137L Expression and Checkpoint Blockade Immunotherapies

In some embodiments, the present disclosure relates to methods of treating or delaying progression of cancer in a subject in need thereof comprising administering an effective amount of a checkpoint blockade immunotherapy to the subject if the level of expression of CD137L in a sample obtained from the subject is higher than a reference level. In some embodiments, the method comprises obtaining a sample from the subject, and measuring the level of expression of CD137L in the sample prior to administration of the checkpoint blockade immunotherapy. In some embodiments, the checkpoint blockade immunotherapy is any one or more of the checkpoint blockade immunotherapies described herein.

In some embodiments, the present disclosure relates to methods of determining whether a subject is likely to respond to a checkpoint blockade immunotherapy. In some embodiments, the method comprises obtaining a sample from the subject, measuring the level of expression of CD137L in the sample, and determining that the subject is likely to respond to the checkpoint blockade immunotherapy when the level of expression of CD137L in the sample is higher than a reference level.

In some embodiments, the present disclosure relates to methods of treating or delaying progression of cancer in a subject in need thereof comprising administering an effective amount of a checkpoint blockade immunotherapy to the subject after it is determined that the subject is likely to respond to the checkpoint blockade immunotherapy. In some embodiments, the checkpoint blockade immunotherapy is any one or more of the checkpoint blockade immunotherapies described herein. In some embodiments, responsiveness of the subject comprises treatment efficacy. In some embodiments, responsiveness of the subject comprises reduced tumor volume. In some embodiments, responsiveness of the subject comprises serological responsiveness.

In some embodiments, the present disclosure relates to methods of selecting a subject having cancer for treatment with a checkpoint blockade immunotherapy. In some embodiments, the method comprises measuring the level of expression of CD137L in a sample obtained from the subject, and selecting the subject for treatment with the checkpoint blockade immunotherapy if the level of expression of CD137L in the sample is higher than a reference level. In some embodiments, the checkpoint blockade immunotherapy is any one or more of the checkpoint blockade immunotherapies described herein.

Checkpoint Blockade Immunotherapy

In some embodiments, the present disclosure relates to the use of a checkpoint blockade immunotherapy. In some embodiments, use of a checkpoint blockade immunotherapy comprises administering to a subject an antibody targeting one or more immune checkpoint pathways (e.g., the PD-1:PD-L1 pathway). Any checkpoint blockade immunotherapy known in the art may be used in the methods of the present disclosure, including, for example, a therapy comprising one or more antibodies targeting CTLA4, PD-1, PD-L1, TIM3, LAG3, CD27, CD28, CD40, OX40, GITR, BTLA, VISTA, B7-H3, B7-H4, DO, and/or KIR. In some embodiments, the checkpoint blockade immunotherapy comprises administering an anti-PD-1 antibody. In some embodiments, the checkpoint blockade immunotherapy comprises administering an anti-PD-L1 antibody. In some embodiments, the checkpoint blockade immunotherapy is used in combination with an anti-CD137 antibody (as described herein).

### Additional Therapeutic Agents

The anti-cancer therapies described herein (e.g., an anti-CD137 antibody, a checkpoint blockade immunotherapy) may be administered alone as monotherapy, or may comprise one or more additional therapeutic agents or therapies. In some embodiments, the one or more (e.g., one or more, two or more, three or more, four or more, five or more, etc.) additional therapeutic agents are one or more of a viral gene therapy, immune checkpoint inhibitors, target therapies, radiation therapies, and/or chemotherapies. In some embodiments, the present disclosure provides a combination therapy, which comprises an anti-cancer therapy described herein (e.g., an anti-CD137 antibody, a checkpoint blockade immunotherapy) in combination with one or more additional therapies or therapeutic agents for separate, sequential or simultaneous administration. The term “additional therapy” or “additional therapeutic agent” may refer to a therapy or therapeutic agent which does not employ the same immunotherapy as is provided in the anti-cancer therapy. In some embodiments, the present disclosure provides a combination therapy for treating cancer in a mammal, which comprises administering to the mammal an effective amount of an anti-cancer therapy of the present disclosure (e.g., an anti-CD137 antibody, a checkpoint blockade immunotherapy) in combination with one or more additional therapeutic agents.

A wide variety of cancer therapeutic agents may be used in combination with a binding molecule provided by the present disclosure. One of ordinary skill in the art will recognize the presence and development of other cancer therapies which can be used in combination with the methods and binding molecules of the present disclosure, and will not be restricted to those forms of therapy set forth herein. Examples of categories of additional therapeutic agents that may be used in the combination therapy for treating cancer include (1) chemotherapeutic agents, (2) immunotherapeutic agents, and (3) hormone therapeutic agents.

The term “chemotherapeutic agent” refers to a chemical or biological substance that can cause death of cancer cells, or interfere with growth, division, repair, and/or function of cancer cells. Examples of chemotherapeutic agents include those that are disclosed in WO 2006/129163, and US 20060153808, the disclosures of which are incorporated herein by reference. Examples of particular chemotherapeutic agents include: (1) alkylating agents, such as chlorambucil (LEUKERAN), mcyclophosphamide (CYTOXAN), ifosfamide (IFEX), mechlorethamine hydrochloride (MUSTARGEN), thiotepa (THIOPLEX), streptozotocin (ZANOSAR), carmustine (BICNU, GLIADEL WAFER), lomustine (CEENU), and dacarbazine (DTIC-DOME); (2) alkaloids or plant vinca alkaloids, including cytotoxic antibiotics, such as doxorubicin (ADRIAMYCIN), epirubicin (ELLENCE, PHARMORUBICIN), daunorubicin (CERUBIDINE, DAUNOXOME), nemorubicin, idarubicin (IDAMYCIN PFS, ZAVEDOS), mitoxantrone (DHAD, NOVANTRONE). dactinomycin (actinomycin D, COSMEGEN), plicamycin (MITHRACIN), mitomycin (MUTAMYCIN), and bleomycin (BLENOXANE), vinorelbine tartrate (NAVELBINE)), vinblastine (VELBAN), vincristine (ONCOVIN), and vindesine (ELDISINE); (3) antimetabolites, such as capecitabine (XELODA), cytarabine (CYTOSAR-U), fludarabine (FLUDARA), gemcitabine (GEMZAR), hydroxyurea (HYDRA), methotrexate (FOLEX, MEXATE, TREXALL), nelarabine (ARRANON), trimetrexate (NEUTREXIN), and pemetrexed (ALIMTA); (4) Pyrimidine antagonists, such as 5-fluorouracil (5-FU); capecitabine (XELODA), raltitrexed (TOMUDEX), tegafur-uracil (UFTORAL), and gemcitabine (GEMZAR); (5) taxanes, such as docetaxel (TAXOTERE), paclitaxel (TAXOL); (6) platinum drugs, such as cisplatin (PLATINOL) and carboplatin (PARAPLATIN), and oxaliplatin (ELOXATIN); (7) topoisomerase inhibitors, such as irinotecan (CAMPTOSAR), topotecan (HYCAMTIN), etoposide (ETOPOPHOS, VEPESSID, TOPOSAR), and teniposide (VUMON); (8) epipodophyllotoxins (podophyllotoxin derivatives), such as etoposide (ETOPOPHOS, VEPESSID, TOPOSAR); (9) folic acid derivatives, such as leucovorin (WELLCOVORIN); (10) nitrosoureas, such as carmustine (BiCNU), lomustine (CeeNU); (11) inhibitors of receptor tyrosine kinase, including epidermal growth factor receptor (EGFR), vascular endothelial growth factor (VEGF), insulin receptor, insulin-like growth factor receptor (IGFR), hepatocyte growth factor receptor (HGFR), and platelet-derived growth factor receptor (PDGFR), such as gefitinib (IRESSA), erlotinib (TARCEVA), bortezomib (VELCADE), imatinib mesylate (GLEEVEC), genefitinib, lapatinib, sorafenib, thalidomide, sunitinib (SUTENT), axitinib, rituximab (RITUXAN, MABTHERA), trastuzumab (HERCEPTIN), cetuximab (ERBITUX), bevacizumab (AVASTIN), and ranibizumab (LUCENTIS), lym-1 (ONCOLYM), antibodies to insulin-like growth factor-1 receptor (IGF-1R) that are disclosed in WO2002/053596); (12) angiogenesis inhibitors, such as bevacizumab (AVASTIN), suramin (GERMANIN), angiostatin, SU5416, thalidomide, and matrix metalloproteinase inhibitors (such as batimastat and marimastat), and those that are disclosed in WO2002055106; and (13) proteasome inhibitors, such as bortezomib (VELCADE).

The term “immunotherapeutic agents” refers to a chemical or biological substance that can enhance an immune response of a mammal. Examples of immunotherapeutic agents include: bacillus Calmette-Guerin (BCG); cytokines such as interferons; vaccines such as MyVax personalized immunotherapy, Onyvax-P, Oncophage, GRNVAC1, Favld, Provenge, GVAX, Lovaxin C, BiovaxID, GMXX, and NeuVax; and antibodies such as alemtuzumab (CAMPATH), bevacizumab (AVASTIN), cetuximab (ERBITUX), gemtuzunab ozogamicin (MYLOTARG), ibritumomab tiuxetan (ZEVALIN), panitumumab (VECTIBIX), rituximab (RITUXAN, MABTHERA), trastuzumab (HERCEPTIN), tositumomab (BEXXAR), ipilimumab (YERVOY) tremelimumab, CAT-3888, agonist antibodies to OX40 receptor (such as those disclosed in WO2009/079335), agonist antibodies to CD40 receptor (such as those disclosed in WO2003/040170, and TLR-9 agonists (such as those disclosed in WO2003/015711, WO2004/016805, and WO2009/022215).

The term “hormone therapeutic agent” refers to a chemical or biological substance that inhibits or eliminates the production of a hormone, or inhibits or counteracts the effect of a hormone on the growth and/or survival of cancerous cells. Examples of such agents suitable for the methods herein include those that are disclosed in US20070117809. Examples of particular hormone therapeutic agents include tamoxifen (NOLVADEX), toremifene (Fareston), fulvestrant (FASLODEX), anastrozole (ARIMIDEX), exemestane (AROMASIN), letrozole (FEMARA), megestrol acetate (MEGACE), goserelin (ZOLADEX), and leuprolide (LUPRON). The binding molecules of this disclosure may also be used in combination with non-drug hormone therapies such as (1) surgical methods that remove all or part of the organs or glands which participate in the production of the hormone, such as the ovaries, the testicles, the adrenal gland, and the pituitary gland, and (2) radiation treatment, in which the organs or glands of the patient are subjected to radiation in an amount sufficient to inhibit or eliminate the production of the targeted hormone.

The combination therapy for treating cancer also encompasses the combination of a binding molecule with surgery to remove a tumor. The binding molecule may be administered to the mammal before, during, or after the surgery.

The combination therapy for treating cancer also encompasses combination of a binding molecule with radiation therapy, such as ionizing (electromagnetic) radiotherapy (e.g., X-rays or gamma rays) and particle beam radiation therapy (e.g., high linear energy radiation). The source of radiation can be external or internal to the mammal. The binding molecule may be administered to the mammal before, during, or after the radiation therapy.

Administering Immunotherapies

In some embodiments, the present disclosure relates to the administration of an effective amount of an anti-cancer therapy (e.g., an anti-CD137 antibody, a checkpoint blockade immunotherapy). In some embodiments, the anti-cancer therapy (e.g., an anti-CD137 antibody, a checkpoint blockade immunotherapy) is used to treat or delay progression of cancer in a subject. In some embodiments, the anti-cancer therapy (e.g., an anti-CD137 antibody, a checkpoint blockade immunotherapy) delays the onset of cancer, including biochemical, histological and/or behavioral symptoms of cancer, its complications and intermediate pathological phenotypes presenting during development of cancer. In some embodiments, the anti-cancer therapy (e.g., an anti-CD137 antibody, a checkpoint blockade immunotherapy) delays development of cancer and/or slows the progression of cancer and/or prolongs survival of the subject.

In some embodiments, the anti-cancer therapy (e.g., an anti-CD137 antibody, a checkpoint blockade immunotherapy) is capable of inhibiting tumor cell growth and/or proliferation. In some embodiments, the tumor cell growth and/or proliferation is inhibited by at least about 5% when contacted with the anti-cancer therapy (e.g., an anti-CD137 antibody, a checkpoint blockade immunotherapy) relative to corresponding tumor cells not contacted with the anti-cancer therapy. For example, the tumor cell growth and/or proliferation is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% when contacted with the anti-cancer therapy (e.g., an anti-CD137 antibody, a checkpoint blockade immunotherapy) relative to corresponding tumor cells not contacted with the anti-cancer therapy. In some embodiments, the tumor cell growth and/or proliferation is inhibited by at least about 1-fold when contacted with the anti-cancer therapy (e.g., an anti-CD137 antibody, a checkpoint blockade immunotherapy) relative to corresponding tumor cells not contacted with the anti-cancer therapy. For example, the tumor cell growth and/or proliferation is inhibited by at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5 fold, at least about 8-fold, at least about 8.5-fold, at least about 9-fold, at least about 9.5-fold, at least about 10-fold, at least about 100-fold, or at least about 1000-fold when contacted with the anti-cancer therapy (e.g., an anti-CD137 antibody, a checkpoint blockade immunotherapy) relative to corresponding tumor cells not contacted with the anti-cancer therapy.

In some embodiments, the anti-cancer therapy (e.g., an anti-CD137 antibody, a checkpoint blockade immunotherapy) is capable of reducing tumor volume in a subject when the subject is administered the anti-cancer therapy. In some embodiments, the anti-cancer therapy (e.g., an anti-CD137 antibody, a checkpoint blockade immunotherapy) is capable of reducing tumor volume in a subject by at least about 5% relative to the initial tumor volume in the subject (e.g., prior to administration of the anti-cancer therapy). For example, the anti-cancer therapy (e.g., an anti-CD137 antibody, a checkpoint blockade immunotherapy) is capable of reducing tumor volume in a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% relative to the initial tumor volume in the subject (e.g., prior to administration of the anti-cancer therapy). In some embodiments, the anti-cancer therapy (e.g., an anti-CD137 antibody, a checkpoint blockade immunotherapy) is capable of reducing tumor volume in a subject by at least about 1-fold relative to the initial tumor volume in the subject (e.g., prior to administration of the anti-cancer therapy). For example, the anti-cancer therapy (e.g., an anti-CD137 antibody, a checkpoint blockade immunotherapy) is capable of reducing tumor volume in a subject by at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5 fold, at least about 8-fold, at least about 8.5-fold, at least about 9-fold, at least about 9.5-fold, at least about 10-fold, at least about 100-fold, or at least about 1000-fold relative to the initial tumor volume in the subject (e.g., prior to administration of the anti-cancer therapy). Methods of monitoring tumor cell growth and/or proliferation, tumor volume, and/or tumor inhibition are known in the art, including, for example, via the methods described in Example 3 below.

In some embodiments, the anti-cancer therapy (e.g., an anti-CD137 antibody, a checkpoint blockade immunotherapy) has therapeutic effect on a cancer. In some embodiments, the anti-cancer therapy (e.g., an anti-CD137 antibody, a checkpoint blockade immunotherapy) reduces one or more signs or symptoms of a cancer. In some embodiments, a subject suffering from a cancer goes into partial or complete remission when administered the anti-cancer therapy (e.g., an anti-CD137 antibody, a checkpoint blockade immunotherapy).

Binding molecules and pharmaceutical compositions of the present disclosure are useful for therapeutic, diagnostic, or other purposes, such as modulating an immune response, treating cancer, enhancing efficacy of other cancer therapy, enhancing vaccine efficacy, or treating autoimmune diseases. In some embodiments, the present disclosure provides methods of treating a disorder in a mammal (e.g., after measuring CD137L expression in a sample taken from the mammal), which comprises administering to the mammal in need of treatment an effective amount of an anti-cancer therapy described herein.

The anti-cancer therapies of the present disclosure (e.g., an anti-CD137 antibody, a checkpoint blockade immunotherapy) may be administered via any suitable enteral or parenteral route of administration. The term “enteral route” of administration may refer to the administration via any part of the gastrointestinal tract. Enteral routes of administration include, for example, oral, mucosal, buccal, rectal, intragastric, etc. The term “Parenteral route” of administration may refer to a route of administration other than enteral route. Parenteral routes of administration include, for example, intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, topical administration, etc. The anti-cancer therapies of the disclosure (e.g., an anti-CD137 antibody, a checkpoint blockade immunotherapy) may be administered using any suitable method, such as by oral ingestion, nasogastric tube, gastrostomy tube, injection, infusion, implantable infusion pump, and osmotic pump. The suitable route and method of administration may vary depending on a number of factors such as the specific antibody being used, the rate of absorption desired, specific formulation or dosage form used, type or severity of the disorder being treated, the specific site of action, and conditions of the patient, and can be readily selected by a person skilled in the art

An effective amount of an anti-cancer therapy of the present disclosure (e.g., an anti-CD137 antibody, a checkpoint blockade immunotherapy) may range from about 0.001 to about 500 mg/kg, including, for example, about 0.01 to about 100 mg/kg, of the body weight of the subject. For example, the amount may be about 0.3 mg/kg, 1 mg/kg, 3 mg/kg, 5 mg/kg, 10 mg/kg, 50 mg/kg, or 100 mg/kg of body weight of the subject. In some embodiments, the effective amount of the anti-cancer therapy (e.g., an anti-CD137 antibody, a checkpoint blockade immunotherapy) is in the range of about 0.01-30 mg/kg of body weight of the subject. In some other embodiments, the effective amount of the anti-cancer therapy (e.g., an anti-CD137 antibody, a checkpoint blockade immunotherapy) is in the range of about 0.05-15 mg/kg of body weight of the subject. The precise dosage level to be administered can be readily determined by a person skilled in the art and will depend on a number of factors, such as the type, and severity of the disorder to be treated, the particular anti-cancer therapy employed, the route of administration, the time of administration, the duration of the treatment, the particular additional therapy employed, the age, sex, weight, condition, general health and prior medical history of the patient being treated, and like factors well known in the medical arts.

An anti-cancer therapy of the present disclosure (e.g., an anti-CD137 antibody, a checkpoint blockade immunotherapy) may be administered on multiple occasions. Intervals between single doses can be, for example, daily, weekly, monthly, every three months or yearly. An exemplary treatment regimen entails administration once per week, once every two weeks, once every three weeks, once every four weeks, once a month, once every three months or once every three to six months. The precise timing of dosages to be administered can be readily determined by a person skilled in the art.

The present disclosure will be more fully understood by reference to the following examples. The examples should not, however, be construed as limiting the scope of the present disclosure. It is understood that the examples and embodiments described herein are for illustrative purposes only, and that various modifications or changes in light thereof will be suggested to persons skilled in the art, and are to be included within the spirit and purview of this application and scope of the appended claims.

### EXAMPLES

### Example 1: CD137L Expression in Multiple Types of Human Cancers

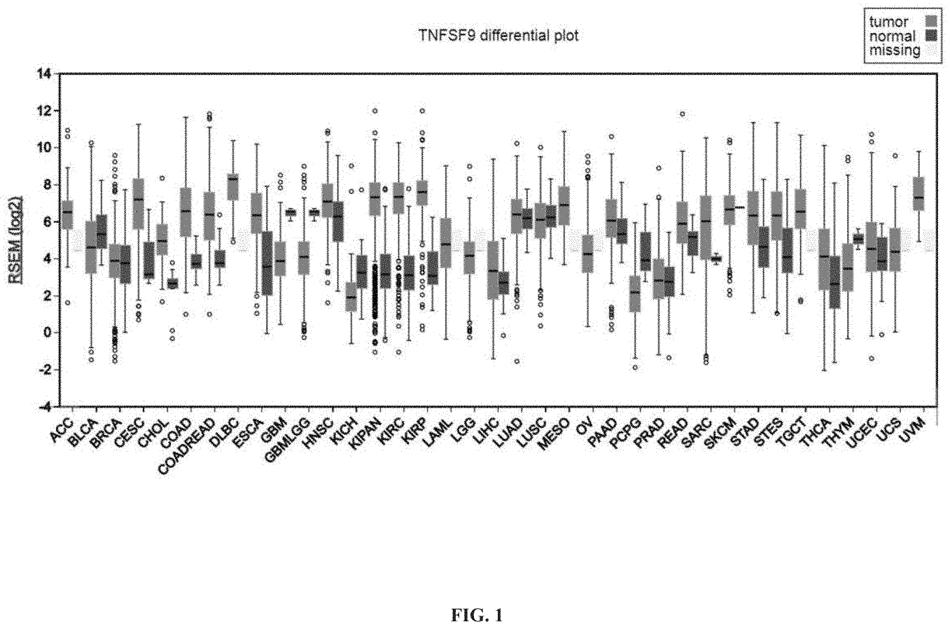

To begin to identify whether an association exists between CD137L expression and cancer, the expression status of CD137L (also known as TNFSF9) in human cancers was checked against The Cancer Genome Atlas (TCGA) database. As shown in FIG. 1, CD137 ligand was frequently overexpressed at the transcript level in multiple human tumor panels when compared to the corresponding matched normal tissues.

### Example 2: Human Cancer Cells Express Functional CD137L

### Methods

To examine whether some human cancer cell express functional CD137L, a cellular NFκB luciferase reporter assay was conducted. Briefly, 293T cells stably expressing an NFκB luciferase reporter were transfected with a DNA construct expressing human CD137, and the cells were co-cultivated with the human B-cell lymphoma cells Daudi or Raji at different ratios. The cell mixture was incubated with serial dilutions of isotype control or ligand-blocking anti-CD137 antibodies overnight, and luciferase activity was measured using the Promega luciferase assay kit according to manufacturer's instructions. Relative luciferase units (RLUs) were calculated vs. the levels of luciferase expressed in 293T cells in the absence of antibody treatment.

Results

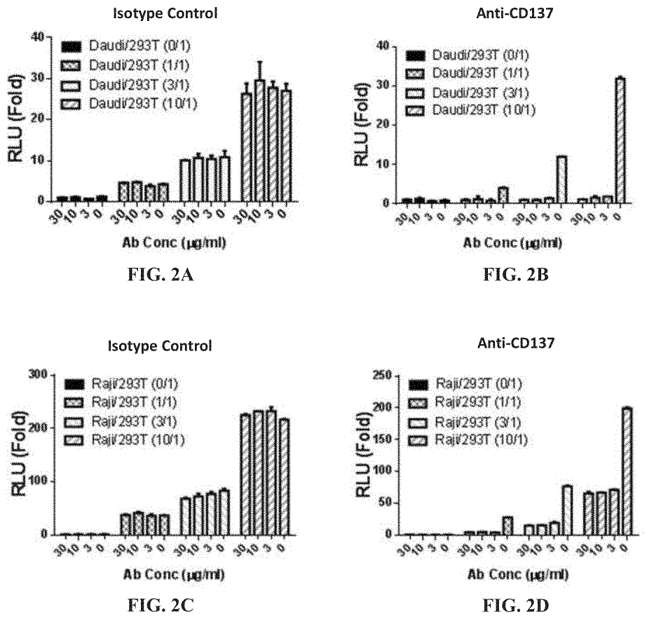

CD137L can signal through CD137 to activate downstream NFκB signaling. To determine whether human cancer cells express functional CD137L, human Daudi and Raji B cell lymphoma cell lines were tested to identify whether these cells could specifically activate CD137-mediated signaling, using a cellular NFκB luciferase reporter assay. As shown in FIGS. 2A and 2C, when Daudi (FIG. 2A) or Raji (FIG. 2C) cells were mixed with the CD137-expressing 293T reporter cells, NFκB signaling was activated in a cell ratio-dependent manner. To exclude the possibility that factors other than the CD137 ligand expressed on the Daudi or Raji cells were stimulating NFκB signaling in the 293T reporter cells, an anti-CD137 antibody capable of blocking CD137 ligand binding was added in this assay. As shown in FIGS. 2B and 2D, the antibody significantly inhibited the NFκB signaling stimulated by both cell types, suggesting that both Daudi and Raji B lymphoma cells express functional CD137L that can trigger NFκB-dependent signaling in neighboring cells through CD137.

### Example 3: CD137 Ligand Expression in Certain Mouse Tumor Cells

Methods

To examine the expression status of CD137L in mouse tumor cells, a series of mouse tumor cell lines (including the liver cancer H22 (CCTCC, CAT #: 3142C0001000000110), colon cancer CT26 (SIBS, CAT #: TCM37) and MC38 (FDCC, CAT #: FDCC-HJC349), melanoma B16F10 (SIBS, CAT #: TCM36), lung cancer LL/2 (SIBS, CAT #: TCM7), pancreatic cancer Pan02 (NIH), prostate cancer RM-1(SIBS, CAT #: TCM14), B lymphoma A20 (ATCC, CAT #: TIB-208), T lymphoma L5178-S(ATCC, CAT #CRL-1723) and L5178-R (ATCC, CAT #CRL-1722) cell lines) were expanded in respective growth medium according to manufacturer's instructions, and were subjected to flow cytometry analysis to detect CD137L expression. Briefly 5×10⁶ cells per cell line (in exponential growth phase) were resuspended in 1 mL FACS buffer (2% BSA in PBS), and 200 μL/well were aliquoted into a 96-well plate. Cells were collected by centrifugation, and the supernatants were discarded. 100 μL/well of a 1:100 dilution of PE-conjugated isotype control antibody or anti-mouse CD137L antibody (BioLegend, CAT #107105) was added to the wells, and the samples were incubated for 30 minutes on ice in the dark. After washing the samples with FACS buffer, cells were resuspended in 200 μL of FACS buffer and were subjected to flow cytometry analysis.

To examine the in vivo anti-tumor responses of anti-CD137 immunotherapy, 10 different mouse syngeneic tumor models that were tested for CD137L surface expression described above were profiled with αCD137-AB1 antibody treatment, in comparison with an isotype control antibody treatment. Briefly, these different mouse tumor cells were maintained in vitro as a monolayer culture in DMEM or RPMI-1640 medium supplemented with 10% fetal bovine serum at 37° C. in an atmosphere of 5% CO₂ in air. The tumor cells were routinely subcultured. The cells growing in an exponential growth phase were harvested and counted for tumor inoculation. Female mice at 7-8 weeks in age were inoculated subcutaneously at the right flank region with corresponding tumor cells in 0.1 ml of PBS for tumor development, 2×10⁵-3×10⁶ cells per inoculation site based on the tumor growth kinetics determined in pilot experiments. Depending on the origin of each tumor cell line, the corresponding mouse strain was used for tumor model development: BALB/c mice were used for the H22, CT26, EMT6, A20 tumor models; C57BL/6 mice were used for the B16F10, MC38, RM-1 and Pan02 tumor models; and DBA/2 mice were used for the L5178-R and L5178-S T lymphoma models. The treatments were started when the mean tumor size reached ˜100 mm³ with isotype control or αCD137-AB1 antibody, 8-10 mice per treatment group. The antibodies were dosed at levels between 10-50 mg/kg through intraperitoneal injection, twice a week for 3 weeks, or until the average group tumor volume reach 2000 mm³. The Pan02 tumor model was examined with the surrogate anti-mouse PD-1 or anti-mouse PD-L1 antibody. Tumor growth inhibition (TGI): TGI(%) was an indication of antitumor effectiveness, and was expressed as: TGI (%)=100×(1−T/C). T and C were the mean tumor volume (or weight) of the treated and control groups, respectively, on a given day.

Results

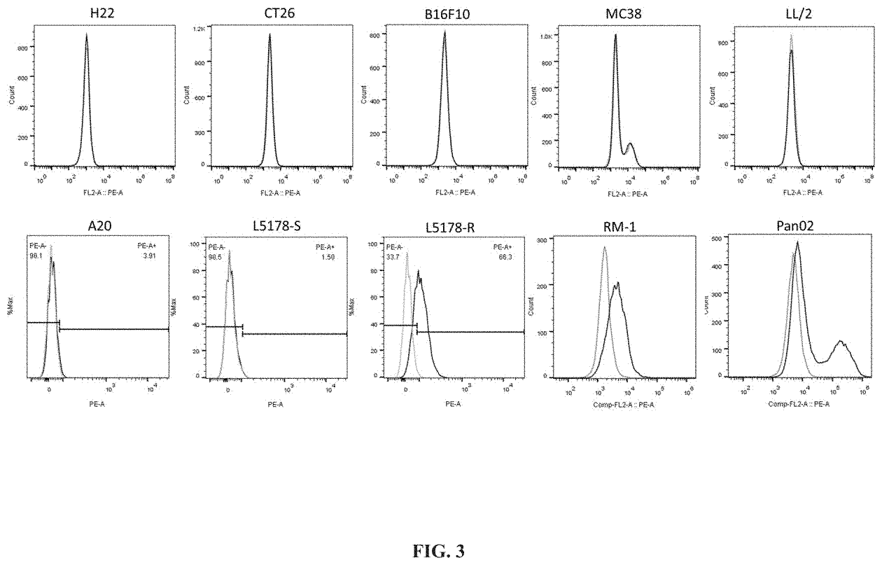

To detect CD137 ligand expression in mouse tumor cells, some commonly used mouse tumor cell lines were profiled by flow cytometry using an anti-mouse CD137 ligand antibody (FIG. 3). Of the ten tumor cell lines tested, six cell lines (H22, CT26, B16F10, LL/2, A20, and L5178-S cells) had little to no detectable CD137L on their surface, while four cells lines had appreciable CD137L levels (MC38, L5178-R, RM-1, and Pan02 cells).

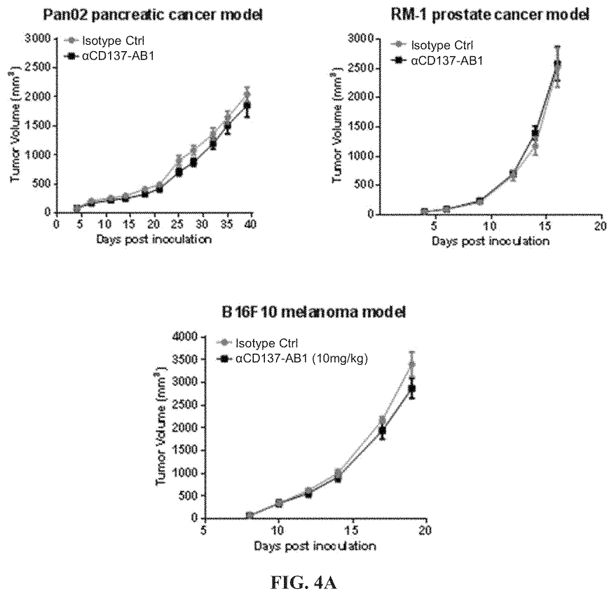

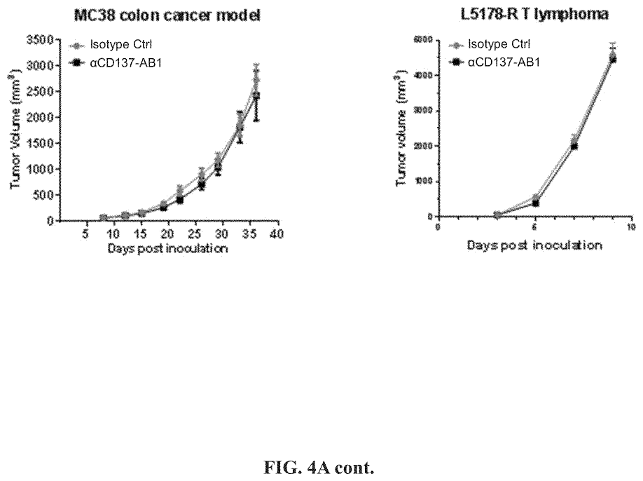

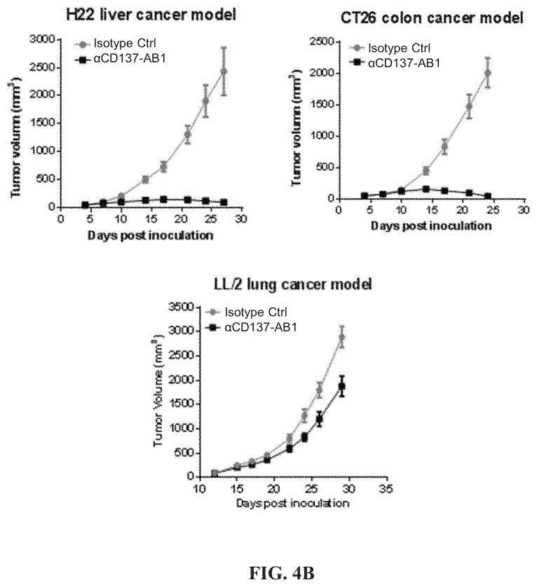

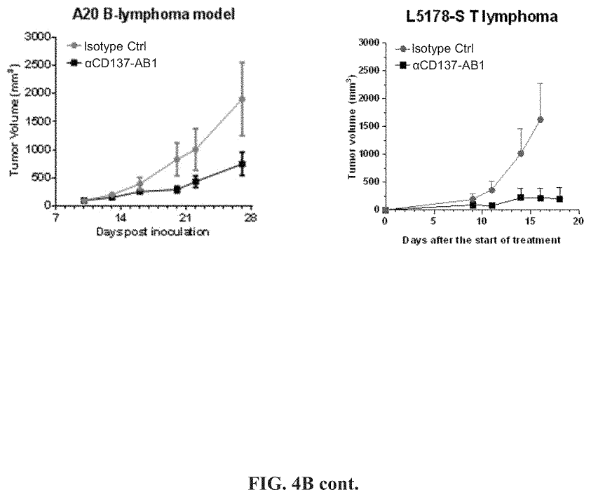

Next, the in vivo anti-tumor activity of targeting CD137 with an agonist antibody that recognizes mouse CD137 (antibody αCD137-AB1) was examined for the 10 syngenic mouse tumor models tested for CD137L surface expression described above (i.e., the H22, CT26, B16F10, LL/2, MC38, A20, L5178-S, L5178-R, RM-1, and Pan02 cell lines). αCD137-AB1 is a fully human IgG4 anti-CD137 monoclonal antibody, which is capable of activating CD137-mediated signaling from human, monkey, and rodent CD137 (See PCT International Application No. PCT/CN2017/098332, incorporated herein by reference in its entirety). The anti-tumor efficacy of αCD137-AB1 for the 10 syngeneic mouse tumor models was profiled (FIG. 4A-B). As shown in FIGS. 4A and 4B, the tumor responses to αCD137-AB1 were categorized into groups of non-responders (tumor growth inhibition (TGI)<20%) and responders (TGI>35%), respectively. Interestingly, an inverse correlation between CD137L expression and responsiveness to the agonist anti-CD137 antibody was observed.

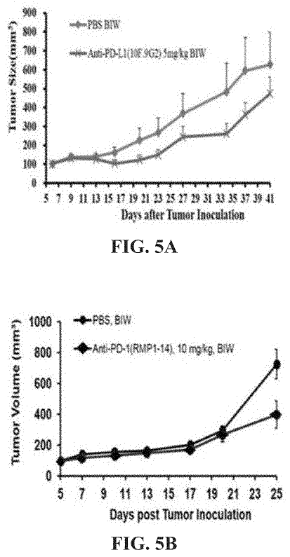

The Pan02 cell line was positive for CD137L expression (FIG. 3) and resistant to anti-CD137 monotherapy in the syngenic mice model (FIG. 4A). However, this same syngenic mouse tumor model was responsive to anti-PD-L1 (Bio X Cell) monotherapy (FIG. 5A) or anti-PD-1 (Bio X Cell) monotherapy (FIG. 5B).

Taken together, the data in the experiments provided above may suggest that CD137L⁺ cancer cells may be resistant to treatment with anti-CD137 antibody therapy, but may be susceptible to checkpoint blockade immunotherapy (e.g., anti-PD-L1 or anti-PD-1 therapy). As such, CD137L expression in tumor cells may serve as a biomarker to negatively stratify patients for enrollment into anti-CD137 antibody therapies and/or positively stratify patients for enrollment into checkpoint blockade immunotherapies (such as anti-PD-1 and/or anti-PD-L1 immunotherapies).

### Example 4: Biomarkers Responding in Different Combination Studies

To further examine the in vivo anti-tumor responses of anti-CD137, 5 different combination studies in mouse syngeneic tumor models that were tested for CD137L surface expression described above were profiled with αCD137-AB1 antibody treatment, in comparison with an isotype control antibody treatment. a) C57BL/6 female mice with established LL/2 mouse lung cancer tumors were treated twice a week through i.p. injection with an isotype control, Anti-CD137, anti-PDL1, or combination of anti-CD137 and anti-PDL1; b) C57BL/6 female mice with established LL/2 mouse lung cancer tumors that ectopically express human CD20, were treated twice a week through i.p. injection with an isotype control, anti-CD137, Rituximab, or combination of anti-CD137 and Rituximab; c) BALB/c female mice with established CT26 mouse colon cancer tumors were treated twice a week through i.p. injection with vehicle, anti-CD137, anti-PD1 (clone RMP1-14), or combination of anti-CD137 and anti-PD1; d) C57BL/6 female mice with established MC38 mouse colon cancer tumors were treated twice a week through i.p. injection with an isotype control, anti-CD137, anti-CTLA4, or combination of anti-CD137 and anti-CTLA4; e) C57BL/6 female mice with established MC38 mouse colon cancer tumors received a single dose (20 Gy) of radiation in the tumor site, followed by twice a week treatment through i.p. injection with anti-CD137, or not.

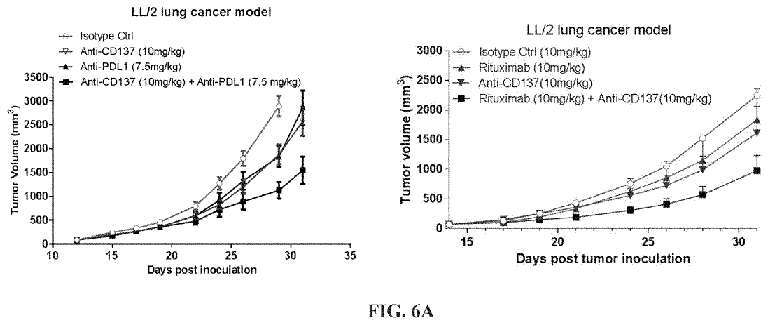

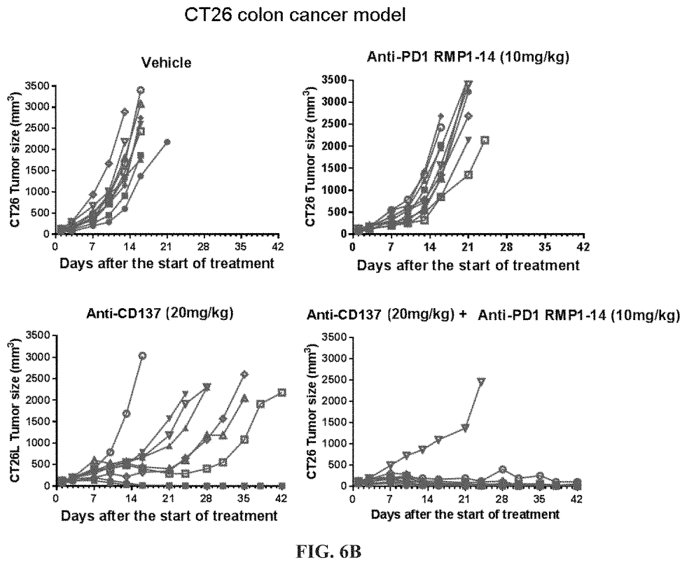

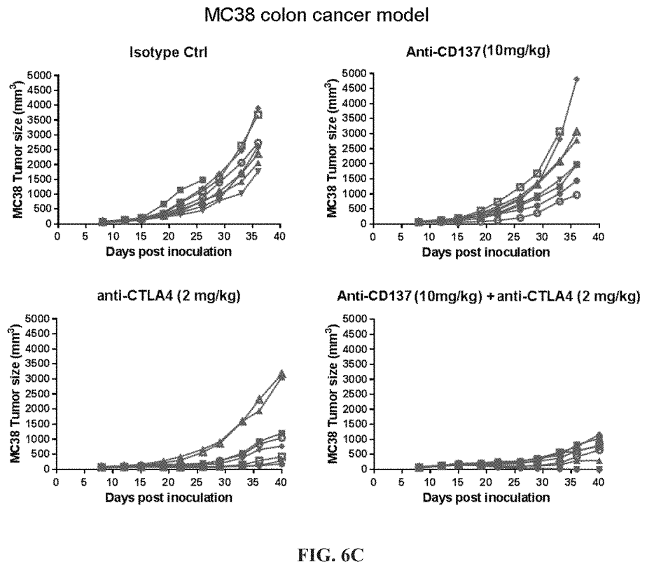

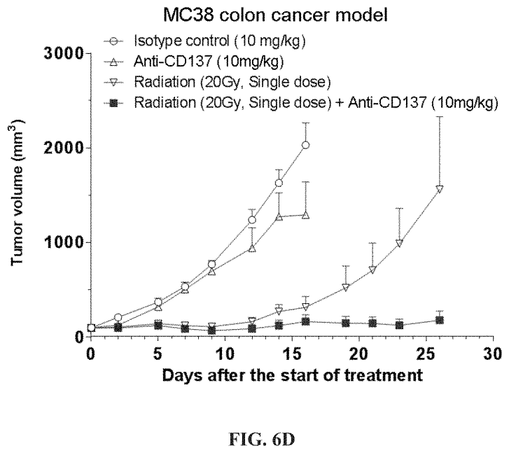

As shown in FIG. 6A-6D, all of anti-CD137 combination studies enhanced the anti-tumor efficacy, indicating that they could be responders even they are CD137L-cancers, even though they are non-responders in monotherapy. As shown in Table 4 below, the CD137L+ cancer cells were resistant to anti-CD137 monotherapy, while >56% (4/7 mouse models tested) of the CD137L-cancers were susceptible to the anti-CD137 monotherapy, but >85% (6/7) of the CD137L-cancers responded in combination studies.

TABLE 4
6/7 > 85% probability positive
biomarkers are indicative of drug response

| Tumor | CD137L | Respond to | Respond to anti-CD137 |
|---|---|---|---|
| model | Expression | anti-CD137 | in Combination study |
| Pan02 | (+) | Non-responder | |
| RM-1 | (+) | Non-responder | |
| L5178-R | (+) | Non-responder | |
| L5178-S | (−) | Responder | |
| H22 | (−) | Responder | |
| CT26 | (−) | Responder | Very strong response in |
| | | | anti-PD1 combination |
| LL/2 | (−) | Weak Responder | Stronger response in |
| | | | anti-PDL1 or |
| | | | anti-CD20 combination |
| A20 | (−) | Responder | |
| B16F10 | (−) | Non-responder | |
| MC38 | (−) | Non-responder | Strong response in |
| | | | anti-CTLA4 or |
| | | | radiation combination |

### Example 5: CD137L IHC Staining in Patient Samples

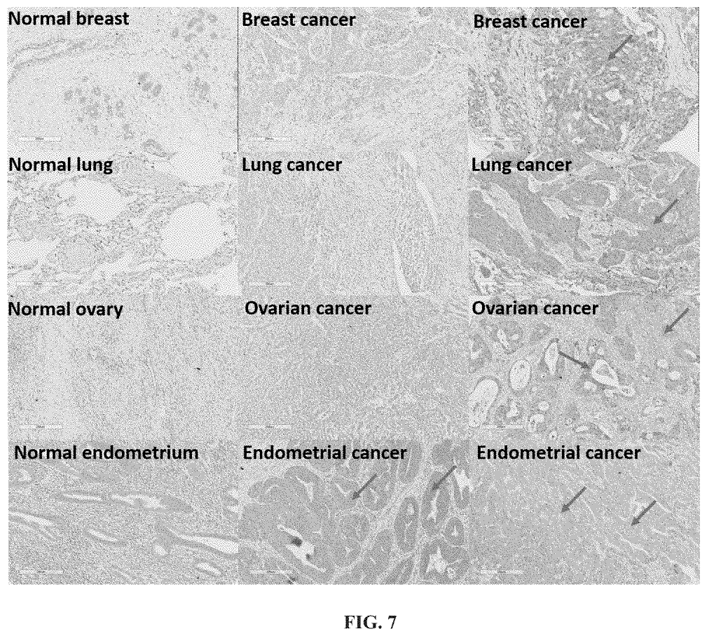

FFPE sections from normal human tissues and tumors from different patients were processed and stained with mouse anti-human CD137L antibody, followed by detection with HRP-labeled anti-mouse secondary antibody and subsequent DAB chromogen. The tissue slides were counterstained with Hematoxylin. The FIG. 7 shows that normal human breast, lung, ovary and endometrium tissues are negative for CD137L staining, but positive expression of CD137L can be detected in tumor tissues from certain patients by IHC staining.

### Example 6: Anti-CD137 is a Competitive Agonist of CD137L

Using ELISA assay, the interaction between CD137 ligand with CD137 in the absence and presence of anti-CD137 antibodies was examined. Recombinant human CD137 proteins were coated onto ELISA microplates to capture biotinylated CD137 ligand in the presence of serial dilutions of anti-CD137 antibodies including αCD137-AB1, AC1097 (IgG2-based antibody with VH and VL domain sequences of SEQ ID NOs:545 and 546, respectively; see Table 2), AC1121 (IgG4-based antibody with VH and VL domain sequences of SEQ ID NOs:547 and 548, respectively; see Table 2), or an isotype control antibody, followed by detection of the captured biotinylated CD137 ligand with a HRP-labeled Neutravidin.

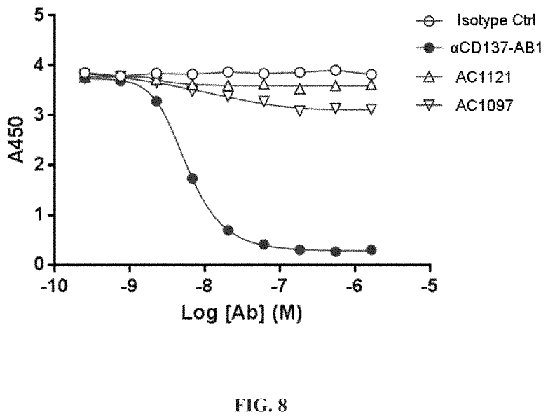

As shown in FIG. 8, αCD137-AB1 blocked CD137 ligand binding to CD137 in a dose-dependent manner, whereas AC1121 did not. AC1097 exhibited only weak ligand blocking activity. As expected, the IgG4 isotype control antibody had no such blocking activity, indicative of assay specificity. These results argue that αCD137-AB1 can act as a potent ligand blocker by disrupting CD137/CD137L interaction, thus inhibiting CD137L stimulated signaling through CD137 receptor, as well as CD137-stimulated CD137L reverse signaling.

1. A method of treating or delaying progression of cancer in a subject in need thereof, the method comprising administering an effective amount of an anti-CD137 antibody to the subject if the level of expression of CD137 ligand (CD137L) in a sample obtained from the subject is lower than a reference level.

2. The method of claim 1, further comprising the steps of:
a) obtaining the sample from the subject; and
b) measuring the level of expression of CD137L in the sample prior to administration of the anti-CD137 antibody to the subject.

3. A method of treating or delaying progression of cancer in a subject in need thereof, the method comprising administering an effective amount of an anti-CD137 antibody to the subject, wherein it has been determined that the subject is likely to respond to the anti-CD137 antibody when the level of expression of CD137L in a sample obtained from the subject is lower than a reference level.

4-5. (canceled)

6. The method of claim 1, wherein the level of expression of CD137L in the sample is below the limit of detection.

7. The method of claim 1, wherein the anti-CD137 antibody binds to an extracellular domain of human CD137, wherein the antibody or the antigen-binding fragment thereof binds to one or more amino acid residues within amino acid residues 34-108 of SEQ ID NO: 531.

8-10. (canceled)

11. The method of claim 1, wherein the anti-CD137 antibody does not bind to one or more of amino acid residues selected from the group consisting of amino acid residues 109-112, 125, 126, 135-138, 150 and 151 of SEQ ID NO: 531.

12. The method of claim 11, wherein the anti-CD137 antibody does not bind to amino acid residues 109-112, 125, 126, 135-138, 150 and 151 of SEQ ID NO: 531.

13. The method of claim 1, wherein the anti-CD137 antibody is cross-reactive with a CD137 polypeptide from at least one non-human species selected from the group consisting of cynomolgus monkey, mouse, rat and dog.

14. (canceled)

15. The method of claim 1, wherein the anti-CD137 antibody is a human antibody.

16. The method of claim 1, wherein the anti-CD137 antibody is an anti-CD137 agonist antibody.

17. The method of claim 1, wherein the anti-CD137 antibody blocks binding of CD137L to CD137.

18. The method of claim 1, wherein the anti-CD137 antibody comprises a human IgG1 or human IgG4 Fc region.

19. The method of claim 1, wherein the anti-CD137 antibody comprises a heavy chain variable region and a light chain variable region,
wherein the heavy chain variable region comprises an HVR-H1 comprising an amino acid sequence selected from the group consisting of SEQ ID NOS: 1-60, an HVR-H2 comprising an amino acid sequence selected from the group consisting of SEQ ID NOS: 61-120, and an HVR-H3 comprising an amino acid sequence selected from the group consisting of SEQ ID NOS: 121-180; and
wherein the light chain variable region comprises an HVR-L1 comprising an amino acid sequence selected from the group consisting of SEQ ID NOS: 181-240, an HVR-L2 comprising an amino acid sequence selected from the group consisting of SEQ ID NOS: 241-300, and an HVR-L3 comprising an amino acid sequence selected from the group consisting of SEQ ID NOS: 301-360.

20. The method of claim 19, wherein the heavy chain variable region comprises an amino acid sequence selected from the group consisting of SEQ ID NOS: 361-420, and wherein the light chain variable region comprises an amino acid sequence selected from the group consisting of SEQ ID NOS: 421-480.

21. The method of claim 20, wherein the anti-CD137 antibody comprises a heavy chain and a light chain, wherein the heavy chain comprises an amino acid sequence selected from the group consisting of SEQ ID NOS: 481-504, and wherein the light chain comprises an amino acid sequence selected from the group consisting of SEQ ID NOS: 505-528.

22. The method of claim 1, wherein the anti-CD137 antibody comprises a heavy chain variable region and a light chain variable region,
(i) wherein the heavy chain variable region comprises an HVR-H1 comprising the amino acid sequence of SEQ ID NO: 15, an HVR-H2 comprising the amino acid sequence of SEQ ID NO: 75, and an HVR-H3 comprising the amino acid sequence of SEQ ID NO: 135; and
wherein the light chain variable region comprises an HVR-L1 comprising the amino acid sequence of SEQ ID NO: 195, an HVR-L2 comprising the amino acid sequence of SEQ ID NO: 255, and an HVR-L3 comprising the amino acid sequence of SEQ ID NO: 315; or
(ii) wherein the heavy chain variable region comprises an HVR-H1 comprising the amino acid sequence of SEQ ID NO: 25, an HVR-H2 comprising the amino acid sequence of SEQ ID NO: 85, and an HVR-H3 comprising the amino acid sequence of SEQ ID NO: 145; and
wherein the light chain variable region comprises an HVR-L1 comprising the amino acid sequence of SEQ ID NO: 205, an HVR-L2 comprising the amino acid sequence of SEQ ID NO: 265, and an HVR-L3 comprising the amino acid sequence of SEQ ID NO: 325; or
(iii) wherein the heavy chain variable region comprises an HVR-H1 comprising the amino acid sequence of SEQ ID NO: 30, an HVR-H2 comprising the amino acid sequence of SEQ ID NO: 90, and an HVR-H3 comprising the amino acid sequence of SEQ ID NO: 150; and
wherein the light chain variable region comprises an HVR-L1 comprising the amino acid sequence of SEQ ID NO: 210, an HVR-L2 comprising the amino acid sequence of SEQ ID NO: 270, and an HVR-L3 comprising the amino acid sequence of SEQ ID NO: 330.

23. The method of claim 22, wherein:
(i) the heavy chain variable region comprises the amino acid sequence of SEQ ID NO: 375, and wherein the light chain variable region comprises the amino acid sequence of SEQ ID NO: 435;
(ii) the heavy chain variable region comprises the amino acid sequence of SEQ ID NO: 385, and wherein the light chain variable region comprises the amino acid sequence of SEQ ID NO: 445; or
(iii) the heavy chain variable region comprises the amino acid sequence of SEQ ID NO: 390, and wherein the light chain variable region comprises the amino acid sequence of SEQ ID NO: 450.

24. The method of claim 23, wherein:
(i) the anti-CD137 antibody comprises a heavy chain and a light chain, wherein the heavy chain comprises the amino acid sequence of SEQ ID NO: 483, and wherein the light chain comprises the amino acid sequence of SEQ ID NO: 507;
(ii) the anti-CD137 antibody comprises a heavy chain and a light chain, wherein the heavy chain comprises the amino acid sequence of SEQ ID NO: 484, and wherein the light chain comprises the amino acid sequence of SEQ ID NO: 508; or
(iii) the anti-CD137 antibody comprises a heavy chain and a light chain, wherein the heavy chain comprises the amino acid sequence of SEQ ID NO: 503, and wherein the light chain comprises the amino acid sequence of SEQ ID NO: 527.

25-30. (canceled)

31. The method of claim 1, further comprising administering to the subject a therapeutically effective amount of at least one additional therapeutic agent.

32. The method of claim 31, wherein the at least one additional therapeutic agent is selected from the group consisting of viral gene therapy, immune checkpoint inhibitors, target therapies, radiation therapies, and chemotherapies.

33. The method of claim 31, wherein the at least one additional therapeutic agent is selected from the group consisting of pomalyst, revlimid, lenalidomide, pomalidomide, thalidomide, a DNA-alkylating platinum-containing derivative, cisplatin, 5-fluorouracil, cyclophosphamide, an anti-CTLA4 antibody, an anti-PD-1 antibody, an anti-PD-L1 antibody, an anti-CD20 antibody, an anti-CD40 antibody, an anti-DRS antibody, an anti-CD1d antibody, an anti-TIM3 antibody, an anti-SLAMF7 antibody, an anti-KIR receptor antibody, an anti-OX40 antibody, an anti-HER2 antibody, an anti-ErbB-2 antibody, an anti-EGFR antibody, cetuximab, rituximab, trastuzumab, pembrolizumab, radiotherapy, single dose radiation, fractionated radiation, focal radiation, whole organ radiation, IL-12, IFNα, GM-CSF, a chimeric antigen receptor, adoptively transferred T cells, an anti-cancer vaccine, and an oncolytic virus.

34. A method of treating or delaying progression of cancer in a subject in need thereof, the method comprising administering an effective amount of a checkpoint blockade immunotherapy to the subject if the level of expression of CD137L in a sample obtained from the subject is higher than a reference level, or wherein it has been determined that the subject is likely to respond to the checkpoint blockade immunotherapy when the level of expression of CD137L in a sample obtained from the subject is higher than a reference level.

35-40. (canceled)

41. The method of claim 1, wherein the subject is a human.

42. The method of claim 1, wherein the sample is a serum sample.

43. The method of claim 1, wherein the sample is a tumor sample.

44. The method of claim 43, wherein the tumor sample is a tumor biopsy.

45. The method of claim 1, wherein the sample comprises one or more cancer cells.

46. The method of claim 1, wherein the level of expression of CD137L is the level of protein expression of CD137L.

47. The method of claim 46, wherein the level of protein expression is measured by a method selected from the group consisting of immunoassay, PET imaging, Western blotting, ELISA, immunohistochemistry, and flow cytometry.

48. The method of claim 1, wherein the level of expression of CD137L is the level of RNA transcript expression of CD137L.

49. The method of claim 48, wherein the level of transcript expression is measured by a method selected from the group consisting of RT-PCR, in situ hybridization, and next generation sequencing.

50. The method of claim 46, wherein the level of expression of CD137L is the level of expression of CD137L by cancer cells.
